(19) United States
(12) Patent Application Publication
(10) Pub. No.: US 2013/0280806 A1
(43) Pub. Date: Oct. 24, 2013

(54) MAMMALIAN GENES INVOLVED IN INFECTION

(71) Applicants: Donald Rubin, Nashville, TN (US); James Murray, Monroe, GA (US); Thomas Hodge, Athens, GA (US)

(72) Inventors: Donald Rubin, Nashville, TN (US); James Murray, Monroe, GA (US); Thomas Hodge, Athens, GA (US)

(21) Appl. No.: 13/698,393

(22) PCT Filed: May 17, 2011
(86) PCT No.: PCT/US11/36873

§ 371 Date: Jun. 28, 2013

Related U.S. Application Data
(60) Provisional application No. 61/345,416, filed on May 17, 2010.

(51) Int. Cl. C12N 15/113

(52) U.S. Cl. CPC C12N 15/1131
USPC 435/375; 435/325

(57) ABSTRACT

The present invention relates to cellular proteins that are involved in infection or are otherwise associated with the life cycle of one or more pathogens.

This application claims the benefit of U.S. Application No. 61/345,416, filed on May 17, 2010, which is hereby incorporated in its entirety by this reference.

### FIELD OF THE INVENTION

The present invention relates to nucleic acid sequences and cellular proteins encoded by these sequences that are involved in infection or are otherwise associated with the life cycle of one or more pathogens, such as a virus, a bacteria, a fungus or a parasite. The invention also relates to modulators of nucleic acid sequences and cellular proteins encoded by these sequences that are involved in infection or are otherwise associated with the life cycle of a pathogen.

### BACKGROUND

Infectious diseases affect the health of people and animals around the world, causing serious illness and death. Black Plague devastated the human population in Europe during the middle ages. Pandemic flu killed millions of people in the 20^{th} century and is a threat to reemerge.

Some of the most feared, widespread, and devastating human diseases are caused by viruses that interfere with normal cellular processes. These include influenza, poliomyelitis, smallpox, Ebola, yellow fever, measles and AIDS, to name a few. Viruses are also responsible for many cases of human disease including encephalitis, meningitis, pneumonia, hepatitis and cervical cancer, warts and the common cold. Furthermore, viruses causing respiratory infections, and diarrhea in young children lead to millions of deaths each year in less-developed countries. Also, a number of newly emerging human diseases such as SARS are caused by viruses. In addition, the threat of a bioterrorist designed pathogen is ever present.

While vaccines have been effective to prevent certain viral infections, relatively few vaccines are available or wholly effective, have inherent risks and tend to be specific for particular conditions. Vaccines are of limited value against rapidly mutating viruses and cannot anticipate emerging viruses or new bioterrorist designed viruses. Currently there is no good answer to these threats.

Traditional treatments for viral infection include pharmaceuticals aimed at specific virus derived proteins, such as HIV protease or reverse transcriptase, or the administration of recombinant (cloned) immune modulators (host derived), such as the interferons. However, the vast majority of viruses lack an effective drug. Those drugs that exist have several limitations and drawbacks that including limited effectiveness, toxicity, and high rates of viral mutations which render antiviral pharmaceuticals ineffective. Thus, an urgent need exists for alternative treatments for viruses and other infectious diseases, and methods of identifying new drugs to combat these threats.

### SUMMARY OF THE INVENTION

The present invention provides genes and gene products set forth in Table 1 that are involved in infection by one or more pathogens such as a virus, a parasite, a bacteria or a fungus, or are otherwise associated with the life cycle of a pathogen. Also provided are methods of decreasing infection in a cell by a pathogen comprising decreasing expression or activity of one or more of these genes or gene products set forth in Table 1. Also provided are methods of decreasing infection by a pathogen in a subject by administering an agent that decreases the expression and/or activity of the genes or gene products set forth in Table 1. Further provided are methods of identifying an agent that decreases infection by a pathogen.

### DETAILED DESCRIPTION OF THE INVENTION

The present invention may be understood more readily by reference to the following detailed description of preferred embodiments of the invention and the Examples included therein.

Before the present compounds, compositions and/or methods are disclosed and described, it is to be understood that this invention is not limited to specific nucleic acids, specific polypeptides, or to particular methods, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. For example, the phrase “optionally obtained prior to treatment” means obtained before treatment, after treatment, or not at all.

As used throughout, by “subject” is meant an individual. Preferably, the subject is a mammal such as a primate, and, more preferably, a human. Non-human primates include marmosets, monkeys, chimpanzees, gorillas, orangutans, and gibbons, to name a few. The term “subject” includes domesticated animals, such as cats, dogs, etc., livestock (for example, cattle, horses, pigs, sheep, goats, etc.), laboratory animals (for example, ferret, chinchilla, mouse, rabbit, rat, gerbil, guinea pig, etc.) and avian species (for example, chickens, turkeys, ducks, pheasants, pigeons, doves, parrots, cockatoos, geese, etc.). The subjects of the present invention can also include, but are not limited to fish (for example, zebrafish, goldfish, tilapia, salmon and trout), amphibians and reptiles.

In the present application, the genes listed in Table 1 are host genes involved in viral infection. All of the host genes involved in viral infection, set forth in Table 1, were identified using gene trap methods that were designed to identify host genes that are necessary for viral infection or growth, but nonessential for cellular survival. These gene trap methods are set forth in the Examples as well as in U.S. Pat. No. 6,448,000 and U.S. Pat. No. 6,777,177. U.S. Pat. Nos. 6,448,000 and 6,777,177 and are both incorporated herein in their entireties by this reference.

As used herein, a gene “nonessential for cellular survival” means a gene for which disruption of one or both alleles results in a cell viable for at least a period of time which allows viral replication to be decreased or inhibited in a cell. Such a decrease can be utilized for preventative or therapeutic uses or used in research. A gene necessary for pathogenic infection or growth means the gene product of this gene, either protein or RNA, secreted or not, is necessary, either directly or indirectly in some way for the pathogen to grow. As utilized throughout, “gene product” is the RNA or protein resulting from the expression of a gene listed in Table 1.

The nucleic acids of these genes and their encoded proteins can be involved in all phases of the viral life cycle including, but not limited to, viral attachment to cellular receptors, viral infection, viral entry, internalization, disassembly of the virus, viral replication, genomic integration of viral sequences, transcription of viral RNA, translation of viral mRNA, transcription of cellular proteins, translation of cellular proteins, trafficking, proteolytic cleavage of viral proteins or cellular proteins, assembly of viral particles, budding, cell lysis and egress of virus from the cells.

Although the genes set forth herein were identified as cellular genes involved in viral infection, as discussed throughout, the present invention is not limited to viral infection. Therefore, any of these nucleic acid sequences and the proteins encoded by these sequences can be involved in infection by any infectious pathogen such as a bacteria, a fungus or a parasite which includes involvement in any phase of the infectious pathogen's life cycle.

As utilized herein, when referring to any one of the genes in Table 1, what is meant is any gene, any gene product, or any nucleic acid (DNA or RNA) associated with that gene name or a pseudonym thereof, as well as any protein, or any protein from any organism that retains at least one activity of the protein associated with the gene name or any pseudonym thereof which can function as a nucleic acid or protein utilized by a pathogen. The nucleic acid or protein sequence can be from or in a cell in a human, a non-human primate, a mouse, a rat, a cat, a dog, a chimpanzee, a horse, a cow, a pig, a sheep, a guinea pig, a rabbit, a zebrafish, a chicken, to name a few.

By way of example, Table 1 refers to PCBP1. Therefore, this is intended to include, but not be limited to, any PCBP1 gene, PCBP1 gene product, for example, a PCBP1 nucleic acid (DNA or RNA) or PCBP1 protein, from any organism that retains at least one activity of PCBP1 and can function as a PCBP1 nucleic acid or protein utilized by a pathogen.

As used herein, a gene is a nucleic acid sequence that encodes a polypeptide under the control of a regulatory sequence, such as a promoter or operator. The coding sequence of the gene is the portion transcribed and translated into a polypeptide (in vivo, in vitro or in situ) when placed under the control of an appropriate regulatory sequence. The boundaries of the coding sequence can be determined by a start codon at the 5′ (amino) terminus and a stop codon at the 3′ (carboxyl) terminus. If the coding sequence is intended to be expressed in a eukaryotic cell, a polyadenylation signal and transcription termination sequence can be included 3′ to the coding sequence.

Transcriptional and translational control sequences include, but are not limited to, DNA regulatory sequences such as promoters, enhancers, and terminators that provide for the expression of the coding sequence, such as expression in a host cell. A polyadenylation signal is an exemplary eukaryotic control sequence. A promoter is a regulatory region capable of binding RNA polymerase and initiating transcription of a downstream (3′ direction) coding sequence. Additionally, a gene can include a signal sequence at the beginning of the coding sequence of a protein to be secreted or expressed on the surface of a cell. This sequence can encode a signal peptide, N-terminal to the mature polypeptide, which directs the host cell to translocate the polypeptide.

Table 1 provides the Entrez Gene numbers for the human genes set forth herein. The information provided under the Entrez Gene numbers listed in Table 1 is hereby incorporated entirely by this reference. One of skill in the art can readily obtain this information from the National Center for Biotechnology Information at the National Library of Medicine (http://www.ncbi.nlm.nih.gov/entrez/query.fcgi?db=gene). By accessing Entrez Gene, one of skill in the art can readily obtain information about every gene listed in Table 1, such as the genomic location of the gene, a summary of the properties of the protein encoded by the gene, expression patterns, function, information on homologs of the gene as well as numerous reference sequences, such as the genomic, mRNA and protein sequences for each gene. Therefore, one of skill in the art can readily obtain sequences, such as genomic, mRNA and protein sequences by accessing information available under the Entrez Gene number provided for each gene. Thus, all of the information readily obtained from the Entrez Gene Nos. set forth herein is also hereby incorporated by reference in its entirety.

Also provided in Table 1 are the GenBank Accession Nos. for at least one example of for at least one example of the mRNA sequence and the GenBank Accession Nos. for the human protein sequence if available. It is noted that there may be multiple isoforms or variants of a gene or protein, and these are also contemplated herein by reference to the gene, even when the specific Accession Number for that isoform or variant is not given. For certain non-protein coding genes, a non-coding RNA is provided, for example, for SNORA molecules. The nucleic acid sequences and protein sequences provided under the GenBank Accession Nos. mentioned herein are hereby incorporated in their entireties by this reference. One of skill in the art would know that the nucleotide sequences provided under the GenBank Accession Nos. set forth herein can be readily obtained from the National Center for Biotechnology Information at the National Library of Medicine (http://www.ncbi.nlm.nih.gov/entrez/query.fcgi?db=nucleotide). Similarly, the protein sequences set forth herein can be readily obtained from the National Center for Biotechnology Information at the National Library of Medicine (http://www.ncbi.nlm.nih.gov/entrez/query.fcgi?db=protein). The nucleic acid sequences and protein sequences provided under the GenBank Accession Nos. mentioned herein are hereby incorporated in their entireties by this reference.

These examples are not meant to be limiting as one of skill in the art would know how to obtain additional sequences for the genes and gene products listed in Table 1 from other species by accessing GenBank or other sequence databases. One of skill in the art would also know how to align the sequences disclosed herein with sequences from other species in order to determine similarities and differences between the sequences set forth in Table 1 and related sequences, for example, by utilizing BLAST. As set forth herein, a nucleic acid sequence for any of the genes set forth in Table 1 can be a full-length wild-type (or native) sequence, a genomic sequence, a variant (for example, an allelic variant or a splice variant), a nucleic acid fragment, a homolog or a fusion sequence that retains the activity of the gene utilized by the pathogen or its encoded gene product.

TABLE I

| | | Human Genbank | Human Genbank | |
|---|---|---|---|---|
| HUGO Gene | Entrez Gene | RNA accession | protein accession | |
| Name | Number | number | number | Aliases |
| PCBP1 | 5093 | NM_006196 | NP_006187 | NRPX; HNRPE1; |
| | | | | hnRNP-X; hnRNP- |
| | | | | E1; PCBP1 |
| AREGB | 727738 | NM_001657 | NP_001092562 | |
| UBXN6 | 80700 | NM_025241 | NP_001164562 | UBXD1; UBXDC2; |
| | | | | FLJ00394; |
| | | | | DKFZp667D109; |
| | | | | UBXN6 |
| CUGBP1 | 10658 | NM_006560 | NP_006551 | CUGBP; NAB50; |
| | | | | CUG-BP; HNAB50; |
| | | | | Brunol2; CUG-BP1; |
| | | | | AA407467; |
| | | | | D2Wsu101e; |
| | | | | 1600010O03Rik; |
| | | | | Cugbp1 |
| PTBP1 | 5725 | NM_002819 | NP_002810 | PTB; PTB2; PTB3; |
| | | | | PTB4; pPTB; |
| | | | | HNRPI; PTB-1; |
| | | | | PTB-T; HNRNPI; |
| | | | | HNRNP-I; |
| | | | | MGC8461; |
| | | | | MGC10830; PTBP1 |
| MATR3 | 9782 | NM_199189 | NP_954659 | MPD2; MGC9105; |
| | | | | KIAA0723; |
| | | | | DKFZp686K0542; |
| | | | | DKFZp686K23100; |
| | | | | MATR3 |
| SNORA74A | 26821 | NR_002915 | | U19; RNU19; |
| | | | | SNORA74A |
| TCF25 | 22980 | NM_014972 | NP_055787 | RGD1309054; |
| | | | | Tcf25 |
| TOB2 | 10766 | NM_016272 | NP_057356 | TOB4; TOBL; |
| | | | | TROB2; TOB2 |
| ECT2 | 1894 | NM_018098 | NP_060568 | FLJ10461; |
| | | | | MGC138291; ECT2 |
| GPR113 | 165082 | NM_001145168 | NP_001138640 | PGR23; |
| | | | | hGPCR37; |
| | | | | FLJ16767; GPR113 |
| SEL1 | 85465 | NM_033505 | NP_277040 | SELI; SEPI; |
| | | | | KIAA1724; EPT1 |
| HIST1H2APS2 | 85303 | | | 2A/T; H2AFTP; |
| | | | | dJ139G21.2; |
| | | | | HIST1H2APS2 |
| PPIA | 5478 | NM_021130 | NP_066953 | CYPA; CYPH; |
| | | | | MGC12404; |
| | | | | MGC23397; |
| | | | | MGC117158; PPIA |
| PAIP2 | 51247 | NM_001033112 | NP_001028284 | PAIP2A; |
| | | | | MGC72018; PAIP2 |
| PHF15 | 23338 | NM_015288 | NP_056103 | JADE2; KIAA0239; |
| | | | | PHF15 |
| SEC24C | 9632 | NM_004922 | NP_004913 | KIAA0079; SEC24C |
| GSTCD | 79807 | NM_001031720 | NP_001026890 | FLJ13273; |
| | | | | DKFZp686I10174; |
| | | | | GSTCD |
| INTS12 | 57117 | NM_001142471 | NP_001135943 | INT12; PHF22; |
| | | | | SBBI22; INTS12 |
| RPS12 | 6206 | NM_001016 | NP_001007 | |
| SNORD101 | 594837 | NR_002434 | | U101; SNORD101 |
| RPL35A | 6165 | NM_000996 | NP_000987 | DBAS; RPL35A |
| IQCG | 84223 | NM_001134435 | NP_001127907 | FLJ11667; |
| | | | | FLJ23571; |
| | | | | FLJ37775; |
| | | | | DKFZp434B227; |
| | | | | IQCG |
| KRT86 | 3892 | NM_002284 | NP_002275 | HB6; Hb1; MNX; |
| | | | | hHb6; KRTHB1; |
| | | | | KRTHB6; |
| | | | | FLJ25176; KRT86 |
| WTAP | 9589 | NM_004906 | NP_004897 | MGC3925; |
| | | | | KIAA0105; |
| | | | | DKFZp686F20131; |
| | | | | WTAP |
| HIST2H2AA3 | 8337 | NM_003516 | NP_003507 | H2A; H2A.2; |
| | | | | H2A/O; H2A/q; |
| | | | | H2AFO; H2a-615; |
| | | | | HIST2H2AA; |
| | | | | HIST2H2AA4; |
| | | | | HIST2H2AA3 |
| HIST2H2BD | 337874 | | | H2B/o; H2B/s; |
| | | | | H2BFO; |
| | | | | HIST2H2BD |
| SNORD58B | 26790 | NR_002572 | | U58b; RNU58B; |
| | | | | SNORD58B |
| SNORD58C | 100124516 | NR_003701 | | |
| SFRS11 | 9295 | NM_004768 | NP_004759 | p54; NET2; |
| | | | | dJ677H15.2; |
| | | | | DKFZp686M13204; |
| | | | | SFRS11 |
| ALS2 | 57679 | NM_001135745 | NP_001129217 | LSJ; PLSJ; IAHSP; |
| | | | | ALS2CR6; |
| | | | | FLJ31851; |
| | | | | KIAA1563; |
| | | | | MGC87187; ALS2 |
| DNAJB9 | 4189 | NM_012328 | NP_036460 | MDG1; ERdj4; |
| | | | | MST049; |
| | | | | MSTP049; |
| | | | | DKFZp564F1862; |
| | | | | DNAJB9 |
| THAP5 | 168451 | NM_001130475 | NP_001123947 | DKFZp313O1132; |
| | | | | THAP5 |
| RN7SL2 | 378706 | NR_027260 | | 7L1C; 7SL1c; |
| | | | | 7L30.1; RNSRP2; |
| | | | | RN7SL2 |
| ARF6 | 382 | NM_001663 | NP_001654 | DKFZp564M0264; |
| | | | | ARF6 |
| BAX | 581 | NM_004324 | NP_004315 | BCL2L4; BAX |
| FTL | 2512 | NM_000146 | NP_000137 | NBIA3; |
| | | | | MGC71996; FTL |
| SNORA73B | 6081 | NR_004406 | | U17B; RNU17B; |
| | | | | SNORA73B |
| RNU105A | 26768 | NR_004404 | | E1c; 105A; |
| | | | | RNU105A |
| HSPA8 | 3312 | NM_006597 | NP_006588 | AP1; HSC54; |
| | | | | HSC70; HSC71; |
| | | | | HSP71; HSP73; |
| | | | | NIP71; HSPA10; |
| | | | | MGC29929; |
| | | | | MGC131511; |
| | | | | HSPA8 |
| KIF12 | 113220 | NM_138424 | NP_612433 | RP11-56P10.3; |
| | | | | KIF12 |
| COL27A1 | 85301 | NM_032888 | NP_116277 | FLJ11895; |
| | | | | KIAA1870; |
| | | | | MGC11337; RP11- |
| | | | | 82I1.1; COL27A1 |
| RAB1A | 5861 | NM_004161 | NP_004152 | RAB1; YPT1; |
| | | | | DKFZp564B163; |
| | | | | RAB1A |
| SCTR | 6344 | NM_002980 | NP_002971 | SR; SCTR |
| KBTBD8 | 84541 | NM_032505 | NP_115894 | TA-KRP; |
| | | | | FLJ57592; |
| | | | | KIAA1842; |
| | | | | KBTBD8 |
| TFPI | 7035 | NM_001032281 | NP_001027452 | EPI; TFI; LACI; |
| | | | | TFPI1; TFPI |
| ZNF827 | 152485 | NM_178835 | NP_849157 | |
| DUSP16 | 80824 | NM_030640 | NP_085143 | MKP7; MKP-7; |
| | | | | KIAA1700; |
| | | | | MGC129701; |
| | | | | MGC129702; |
| | | | | DUSP16 |
| RNLS | 55328 | NM_001031709 | NP_001026879 | C10orf59; |
| | | | | FLJ11218; |
| | | | | RENALASE; RNLS |
| HPSE2 | 60495 | NM_001166244 | NP_001159716 | HPA2; HPR2; |
| | | | | FLJ11684; |
| | | | | FLJ44022; |
| | | | | MGC133234; |
| | | | | HPSE2 |
| ARF4 | 378 | NM_001660 | NP_001651 | ARF2; ARF4 |
| FOSL2 | 2355 | NM_005253 | NP_005244 | FRA2; FLJ23306; |
| | | | | FOSL2 |
| C16ORF62 | 57020 | NM_020314 | NP_064710 | LJ21040; |
| | | | | MGC16824; |
| | | | | DKFZp313M0539; |
| | | | | DKFZp434B0212; |
| | | | | C16orf62 |
| GDE1 | 51573 | NM_016641 | NP_057725 | MIR16; 363E6.2; |
| | | | | GDE1 |
| ZNF581 | 51545 | NM_016535 | NP_057619 | HSPC189; |
| | | | | FLJ22550; ZNF581 |
| TRIB1 | 10221 | NM_025195 | NP_079471 | C8FW; GIG2; |
| | | | | TRB1; SKIP1; |
| | | | | TRIB1 |
| NFE2L1 | 4779 | NM_003204 | NP_003195 | NRF1; TCF11; |
| | | | | LCR-F1; FLJ00380; |
| | | | | NFE2L1 |
| RPL22L1 | 200916 | NM_001099645 | NP_001093115 | MGC104449; |
| | | | | RPL22L1 |
| RAB9B | 51209 | NM_016370 | NP_057454 | RAB9L; RAB9B |
| SYNE2 | 23224 | NM_015180 | NP_055995 | NUA; EDMD5; |
| | | | | NUANCE; SYNE- |
| | | | | 2; FLJ11014; |
| | | | | FLJ43727; |
| | | | | FLJ45710; |
| | | | | FLJ46790; |
| | | | | KIAA1011; |
| | | | | Nesprin-2; |
| | | | | DKFZp434H2235; |
| | | | | DKFZp686H1931; |
| | | | | DKFZp686E01115; |
| | | | | SYNE2 |
| HNF1B | 6928 | NM_000458 | NP_000449 | FJHN; HNF2; |
| | | | | LFB3; TCF2; |
| | | | | HPC11; LF-B3; |
| | | | | MODY5; VHNF1; |
| | | | | HNF1beta; HNF1B |
| ALDOA | 226 | NM_000034 | NP_000025 | ALDA; GSD12; |
| | | | | MGC10942; |
| | | | | MGC17716; |
| | | | | MGC17767; |
| | | | | ALDOA |
| CDKN1B | 1027 | NM_004064 | NP_004055 | KIP1; MEN4; |
| | | | | CDKN4; MEN1B; |
| | | | | P27KIP1; CDKN1B |
| TBCK | 93627 | NM_001163435 | NP_001156907 | TBCKL; HSPC302; |
| | | | | MGC16169; TBCK |
| PARD6B | 84612 | NM_032521 | NP_115910 | PAR6B; PARD6B |
| TMBIM6 | 7009 | NM_001098576 | NP_001092046 | BI-1; TEGT; |
| | | | | BAXI1; TMBIM6 |
| FAM192A | 80011 | NM_024946 | NP_079222 | CDA10; NIP30; |
| | | | | CDA018; C16orf94; |
| | | | | FLJ21799; |
| | | | | MGC74898; |
| | | | | FAM192 |
| RSPRY1 | 89970 | NM_133368 | NP_588609 | KIAA1972; |
| | | | | RSPRY1 |
| BANF1 | 8815 | NM_001143985 | NP_001137457 | BAF; BCRP1; |
| | | | | D14S1460; |
| | | | | MGC111161; |
| | | | | BANF1 |
| EIF1AD | 84285 | NM_032325 | NP_115701 | MGC11102; |
| | | | | EIF1AD |
| ARPC5L | 81873 | NM_030978 | NP_112240 | ARC16-2; |
| | | | | MGC3038; |
| | | | | ARPC5L |
| PAFAH1B1 | 5048 | NM_000430 | NP_000421 | MDS; LIS1; LIS2; |
| | | | | MDCR; PAFAH; |
| | | | | PAFAH1B1 |
| BAT2L | 84726 | NM_013318 | NP_037450 | BAT2L; LQFBS-1; |
| | | | | KIAA0515; |
| | | | | MGC10526; RP11- |
| | | | | 334J6.1; |
| | | | | DKFZp781F05101; |
| | | | | DKFZp781K12107; |
| | | | | BAT2L1 |
| TOR1AIP2 | 163590 | NM_022347 | NP_071742 | NET9; LULL1; |
| | | | | IFRG15; FLJ77012; |
| | | | | MGC120074; |
| | | | | MGC120075; |
| | | | | MGC120076; |
| | | | | MGC120077; |
| | | | | MGC126581; |
| | | | | MGC138430; |
| | | | | RP11-12M5.5; |
| | | | | TOR1AIP2 |
| IFRG15 | 64163 | NM_022347 | NP_071742 | NET9; LULL1; |
| | | | | IFRG15; FLJ77012; |
| | | | | MGC120074; |
| | | | | MGC 120075; |
| | | | | MGC120076; |
| | | | | MGC120077; |
| | | | | MGC126581; |
| | | | | MGC138430; |
| | | | | RP11-12M5.5; |
| | | | | TOR1AIP2 |
| MIR7-1 | 407043 | NR_029605 | | MIRN7-1; mir-7-1; |
| | | | | hsa-mir-7-1; MIR7- |
| | | | | 1 |
| GART | 2618 | NM_000819 | NP_000810 | AIRS; GARS; |
| | | | | PAIS; PGFT; |
| | | | | PRGS; GARTF; |
| | | | | MGC47764; GART |
| SON | 6651 | NM_032195 | NP_115571 | SON3; BASS1; |
| | | | | DBP-5; NREBP; |
| | | | | C21orf50; |
| | | | | FLJ21099; |
| | | | | FLJ33914; |
| | | | | KIAA1019; SON |
| STK35 | 140901 | NM_080836 | NP_543026 | CLIK1; STK35L1; |
| | | | | STK35 |
| RBM5 | 10181 | NM_005778 | NP_543026 | G15; H37; RMB5; |
| | | | | LUCA15; |
| | | | | FLJ39876; RBM5 |
| HNRNPUL2 | 221092 | NM_001079559 | NP_001073027 | HNRPUL2; |
| | | | | DKFZp762N1910; |
| | | | | HNRNPUL2 |
| TTC9C | 283237 | NM_173810 | NP_776171 | MGC29649; |
| | | | | TTC9C |
| RAB5C | 5878 | NM_004583 | NP_004574 | RABL; RAB5CL; |
| | | | | MGC117217; |
| | | | | MGC138857; |
| | | | | RAB5C |
| C4ORF34 | 201895 | NM_174921 | NP_777581 | FLJ13289; C4orf34 |
| EIF4B | 1975 | NM_001417 | NP_001408 | EIF-4B; PRO1843; |
| | | | | EIF4B |
| SFXN2 | 118980 | NM_178858 | NP_849189 | |
| ARL3 | 403 | NM_004311 | NP_004302 | ARFL3; ARL3 |
| FTSJ1 | 24140 | NM_012280 | NP_036412 | JM23; MRX9; |
| | | | | SPB1; TRM7; |
| | | | | CDLIV; MRX44; |
| | | | | FTSJ1 |
| TGIF1 | 7050 | NM_003244 | NP_003235 | HPE4; TGIF; |
| | | | | MGC5066; |
| | | | | MGC39747; TGIF1 |
| SNRPD3 | 6634 | NM_004175 | NP_004166 | SMD3; Sm-D3; |
| | | | | SNRPD3 |
| C22ORF13 | 83606 | NM_031444 | NP_113632 | LLN4; MGC1842; |
| | | | | C22orf13 |
| RPL35 | 296709 | NM_212511 | NP_997676 | MGC72958; Rpl35 |
| HNRNPF | 3185 | NM_001098204 | NP_001091674 | HNRPF; mcs94-1; |
| | | | | MGC110997; |
| | | | | OK/SW-cl.23; |
| | | | | HNRNPF |
| RPL7 | 6129 | NM_000971 | NP_000962 | humL7-1; |
| | | | | MGC117326; RPL7 |
| PDE9A | 5152 | NM_001001567 | NP_001001567 | FLJ90181; |
| | | | | HSPDE9A2; |
| | | | | PDE9A |
| NUDCD1 | 84955 | NM_001128211 | NP_001121683 | CML66; OVA66; |
| | | | | FLJ14991; |
| | | | | NUDCD1 |
| PKHD1L1 | 93035 | NM_177531 | NP_803875 | PKHDL1; |
| | | | | DKFZp586C1021; |
| | | | | PKHD1L1 |
| MAPK6PS5 | 286156 | | | |
| HIST2H2AB | 317772 | NM_175065 | NP_778235 | |
| BOLA1 | 51027 | NM_016074 | NP_057158 | CGI-143; |
| | | | | MGC75015; RP11- |
| | | | | 196G18.18; BOLA1 |
| AGAP1 | 116987 | NM_001037131 | NP_001032208 | GGAP1; CENTG2; |
| | | | | KIAA1099; |
| | | | | MGC71657; |
| | | | | AGAP1 |
| STAU2 | 27067 | NM_001164380 | NP_001157852 | 9K2; 39K3; |
| | | | | MGC119606; |
| | | | | DKFZp781K0371; |
| | | | | STAU2 |
| HSP90AB1 | 3326 | NM_007355 | NP_031381 | HSPC2; HSPCB; |
| | | | | D6S182; HSP90B; |
| | | | | FLJ26984; HSP90- |
| | | | | BETA; HSP90AB1 |
| SLCO1A2 | 6579 | NM_021094 | NP_066580 | OATP; OATP-A; |
| | | | | OATP1A2; |
| | | | | SLC21A3; |
| | | | | SLCO1A2 |
| PCID2 | 55795 | NM_001127202 | NP_001120674 | 10; FLJ11305; |
| | | | | FLJ99362; |
| | | | | MGC16774; RP11- |
| | | | | 98F14.6; |
| | | | | DKFZp686C20226; |
| | | | | PCID2 |
| RPL26 | 6154 | NM_000987 | NP_000978 | |
| ITPRIP | 85450 | NM_033397 | NP_203755 | DANGER; KIAA1754; |
| | | | | bA127L20; |
| | | | | bA127L20.2; RP11- |
| | | | | 127L20.4; ITPRIP |
| CTNND1 | 1500 | NM_001085458 | NP_001078927 | CAS; p120; |
| | | | | CTNND; P120CAS; |
| | | | | P120CTN; |
| | | | | KIAA0384; |
| | | | | CTNND1 |
| SF3A2 | 8175 | NM_007165 | NP_009096 | PRP11; SAP62; |
| | | | | PRPF11; SF3a66; |
| | | | | SF3A2 |
| PLEKHJ1 | 55111 | NM_018049 | NP_060519 | GNRPX; FLJ10297; |
| | | | | PLEKHJ1 |
| MIR1227 | 100302283 | NR_031596 | | MIRN1227; hsa- |
| | | | | mir-1227; MIR1227 |
| PPP1R9A | 55607 | NM_001166160 | NP_001159632 | NRB1; NRBI; |
| | | | | FLJ20068; |
| | | | | KIAA1222; |
| | | | | Neurabin-I; |
| | | | | PPP1R9A |
| HIPK3 | 10114 | NM_001048200 | NP_001041665 | PKY; YAK1; |
| | | | | DYRK6; FIST3; |
| | | | | HIPK3 |
| ASB6 | 140459 | NM_017873 | NP_060343 | MGC1024; |
| | | | | FLJ20548; ASB6 |
| SENP3 | 26168 | NM_015670 | NP_056485 | SSP3; SMT3IP1; |
| | | | | DKFZp762A152; |
| | | | | DKFZp586K0919; |
| | | | | SENP3 |
| TNFSF12 | 8742 | NM_003809 | NP_003800 | APO3L; DR3LG; |
| | | | | TWEAK; |
| | | | | MGC20669; MGC129581; |
| | | | | TNFSF12 |
| TNFSF13 | 8741 | NM_003808 | NP_003799 | APRIL; CD256; |
| | | | | TALL2; TRDL-1; |
| | | | | ligand; |
| | | | | UNQ383/PRO715; |
| | | | | TNFSF13 |
| TNFSF12- | 407977 | NM_172089 | NP_742086 | WE-PRIL; |
| TNFSF13 | | | | TNFSF12- |
| | | | | TNFSF13 |
| MIR505 | 574508 | NR_030230 | | MIRN505; hsa-mir- |
| | | | | 505; MIR505 |
| PANK1 | 53354 | NM_138316 | NP_612189 | PANK; PANK1a; |
| | | | | PANK1b; |
| | | | | MGC24596; |
| | | | | PANK1 |
| MIR107 | 406901 | NR_029524 | | MIRN107; miR- |
| | | | | 107; MIR107 |
| ACTN4 | 81 | NM_004924 | NM_004924 | FSGS; FSGS1; |
| | | | | ACTININ-4; |
| | | | | DKFZp686K23158; |
| | | | | ACTN4 |
| NUMA1 | 4926 | NM_006185 | NP_006176 | NUMA; NUMA1 |
| TBL1XR1 | 79718 | NM_024665 | NP_078941 | C21; DC42; IRA1; |
| | | | | TBLR1; FLJ12894; |
| | | | | TBL1XR1 |
| PA2G4 | 5036 | NM_006191 | NP_006182 | EBPl; HG4-1; p38- |
| | | | | 2G4; PA2G4 |
| FLRT3 | 23767 | NM_013281 | NP_037413 | |
| SLC25A37 | 51312 | NM_016612 | NP_057696 | MSC; MFRN; |
| | | | | MSCP; HT015; |
| | | | | PRO1278; |
| | | | | PRO1584; |
| | | | | PRO2217; |
| | | | | SLC25A37 |
| RPS8 | 6202 | NM_001012 | NP_001003 | |
| SNORD38A | 94162 | NR_001456 | | U38A; RNU38A; |
| | | | | SNORD38A |
| SNORD38B | 94163 | NR_001457 | | U38B; RNU38B; |
| | | | | SNORD38B |
| SNORD46 | 94161 | NR_000024 | | U40; U46; RNU40; |
| | | | | RNU46; SNORD46 |
| SNORD55 | 26811 | NR_000015 | | U39; U55; RNU39; |
| | | | | RNU55; SNORD39; |
| | | | | SNORD55 |
| RBM3 | 5935 | NM_006743 | NP_006734 | RNPL; IS1-RNPL; |
| | | | | RBM3 |
| RANGAP1 | 5905 | NM_002883 | NP_002874 | SD; Fug1; |
| | | | | KIAA1835; |
| | | | | MGC20266; |
| | | | | RANGAP1 |
| BMPR2 | 659 | NM_001204 | NP_001195 | BMR2; PPH1; |
| | | | | BMPR3; BRK-3; T- |
| | | | | ALK; BMPR-II; |
| | | | | FLJ41585; |
| | | | | FLJ76945; BMPR2 |
| DHX9 | 1660 | NM_001357 | NP_001348 | LKP; RHA; DDX9; |
| | | | | NDH2; NDHII; |
| | | | | DHX9 |
| SREBF2 | 6721 | NM_004599 | NP_004590 | REBP2; bHLHd2; |
| | | | | SREBF2 |
| MARCH7 | 64844 | NM_022826 | NP_073737 | AXO; AXOT; |
| | | | | RNF177; MARCH- |
| | | | | VII; |
| | | | | DKFZp586F1122; |
| | | | | MARCH7 |
| RPL29 | 6159 | NM_000992 | NP_000983 | HIP; HUMRPL29; |
| | | | | MGC88589; RPL29 |
| RPL29P10 | 100270973 | | | RPL29_3_370; |
| | | | | RPL29P10 |
| CSRP1 | 1465 | NM_001144773 | NP_001138245 | CRP; CRP1; CSRP; |
| | | | | CYRP; D1S181E; |
| | | | | DKFZp686M148; |
| | | | | CSRP1 |
| AP1G1 | 164 | NM_001030007 | NP_001025178 | ADTG; CLAPG1; |
| | | | | MGC18255; AP1G1 |
| AHCY | 191 | NM_000687 | NP_000678 | SAHH; AHCY |
| CDH6 | 1004 | NM_004932 | NP_004923 | KCAD; CDH6 |
| PABPN1 | 8106 | NM_004643 | NP_004634 | OPMD; PAB2; |
| | | | | PABP2; PABPN1 |
| PPP1R3E | 90673 | NR_026862 | | KIAA1443; |
| | | | | PPP1R3E |
| NPAS2 | 4862 | NM_002518 | NP_002509 | MOP4; PASD4; |
| | | | | bHLHe9; FLJ23138; |
| | | | | MGC71151; NPAS2 |
| APBB1IP | 54518 | NM_019043 | NP_061916 | RIAM; INAG1; |
| | | | | PREL1; RARP1; |
| | | | | APBB1IP |
| SETD5 | 55209 | NM_001080517 | NP_001073986 | FLJ10707; |
| | | | | KIAA1757; |
| | | | | DKFZp686J18276; |
| | | | | SETD5NM_001080517 |
| PARG | 8505 | NM_003631 | NP_003622 | PARG99; |
| | | | | FLJ60456; PARG |
| DR1 | 1810 | NM_001938 | NP_001929 | NC2; NC2-BETA; |
| | | | | DR1 |
| LEPREL1 | 55214 | NM_001134418 | NP_001127890 | P3H2; MLAT4; |
| | | | | FLJ10718; |
| | | | | LEPREL1 |
| DIAPH3 | 81624 | NM_001042517 | NP_001035982 | DRF3; diap3; |
| | | | | mDia2; FLJ34705; |
| | | | | DKFZp434C0931; |
| | | | | DKFZp686A13178; |
| | | | | DIAPH3 |
| SRRM1P | 401475 | | | SRRM1L; SRRM1P |
| CPS1 | 1373 | NM_001122633 | NP_001116105 | |
| PKM2 | 5315 | NM_002654 | NP_002645 | PK3; PKM; TCB; |
| | | | | OIP3; CTHBP; |
| | | | | THBP1; MGC3932; |
| | | | | PKM2 |
| SEMA3F | 6405 | NM_004186 | NP_004177 | SEMA4; SEMAK; |
| | | | | SEMA-IV; |
| | | | | SEMA3F |
| C20ORF111 | 51526 | NM_016470 | NP_057554 | Perit1; HSPC207; |
| | | | | dJ1183121.1; |
| | | | | C20orf111 |
| MANBAL | 63905 | NM_001003897 | NP_001003897 | |
| LIMCH1 | 22998 | NM_001112717.1 | NP_001106188.1 | LMO7B; |
| | | NM_001112718.1 | NP_001106189.1 | LIMCH1A; |
| | | NM_001112719.1 | NP_001106190.1 | MGC72127; |
| | | NM_001112720.1 | NP_001106191.1 | DKFZp434I0312; |
| | | NM_014988.2 | NP_055803.2 | DKFZp686B2470; |
| | | | | DKFZp686G2094; |
| | | | | DKFZp781C1754; |
| | | | | DKFZp781I1455; |
| | | | | DKFZp686A01247; |
| | | | | DKFZp686G18243 |
| PFAS | 5198 | NM_012393.2 | NP_036525.1 | PURL; FGAMS; |
| | | | | FGARAT; |
| | | | | KIAA0361 |
| C17ORF68 | 80169 | NM_025099.5 | NP_079375.3 | CTC1; AAF132; |
| | | | | AAF-132; |
| | | | | FLJ22170; |
| | | | | MGC133331 |
| TRNAI-AAU | 100126481 | | | |
| TRNAS-AGA | 100126489 | | | |
| TRNAT-AGU | 100126497 | | | |
| CSGALNACT2 | 55454 | NM_018590.3 | NP_061060.3 | CHGN2; PRO0082; |
| | | | | FLJ43310; |
| | | | | GALNACT2; |
| | | | | MGC40204; |
| | | | | GALNACT-2; |
| | | | | DKFZp686H13226 |
| FLVCR2 | 55640 | NM_017791.2 | NP_060261.2 | CCT; EPV; PVHH; |
| | | | | MFSD7C; C14orf58; |
| | | | | FLJ20371; |
| | | | | FLVCRL14q |
| HSPE1 | 3336 | NM_002157.2 | NP_002148.1 | CPN10; GROES; |
| | | | | HSP10 |
| RPL27 | 6155 | NM_000988.3 | NP_000979.1 | |
| SNORD12 | 692057 | NR_003030.1 | | HBII-99 |
| SNORD12B | 100113393 | NR_003695.1 | | |
| SNORD12C | 26765 | NR_002433.1 | | E2; E3; E2-1; U106; |
| | | | | RNU106; |
| | | | | SNORD106 |
| MIR1259 | 100302194 | NR_031660.1 | | MIRN1259; hsa- |
| | | | | mir-1259 |
| ZNFX1 | 57169 | NM_021035.2 | NP_066363.1 | FLJ39275; |
| | | | | MGC131926 |
| CBX5 | 23468 | NM_001127321.1 | NP_001120793.1 | HP1; HP1A |
| | | NM_001127322.1 | NP_001120794.1 | |
| | | NM_012117.2 | NP_036249.1 | |
| HNRNPA1 | 3178 | NM_002136.2 | NP_002127.1 | HNRPA1; hnRNP |
| | | NM_031157.2 | NP_112420.1 | A1; hnRNP-A1; |
| | | | | MGC102835 |
| SLC1A3 | 6507 | NM_001166695.1 | NP_001160167.1 | EA6; EAAT1; |
| | | NM_001166696.1 | NP_001160168.1 | GLAST; GLAST1; |
| | | NM_004172.4 | NP_004163.3 | FLJ25094 |
| WDR25 | 79446 | NM_001161476.1 | NP_001154948.1 | MGC4645 |
| | | NM_024515.4 | NP_078791.3 | |
| STEAP4 | 79689 | NM_024636.2 | NP_078912.2 | TIARP; STAMP2; |
| | | | | TNFAIP9; |
| | | | | FLJ23153; |
| | | | | DKFZp666D049 |
| NPTX1 | 4884 | NM_002522.3 | NP_002513.2 | NP1; MGC105123; |
| | | | | DKFZp686J2446 |
| PTPRM | 5797 | NM_001105244.1 | NP_001098714.1 | RPTPM; RPTPU; |
| | | NM_002845.3 | NP_002836.3 | PTPRL1; hR-PTPu; |
| | | | | R-PTP-MU; |
| | | | | MGC166994 |
| RAB30 | 27314 | NM_014488.3 | NP_055303.2 | |
| SNORA70 | 26778 | NR_000011.1 | | U70; RNU70; |
| | | | | DXS648E |
| RNF128 | 79589 | NM_024539.3 | NP_078815.3 | GRAIL; FLJ23516 |
| | | NM_194463.1 | NP_919445.1 | |
| RPS10 | 6204 | NM_001014.3 | NP_001005.1 | DBA9; MGC88819 |
| GNAL | 2774 | NM_001142339.1 | NP_001135811.1 | |
| | | NM_002071.3 | NP_002062.1 | |
| | | NM_182978.2 | NP_892023.1 | |
| CHMP1B | 57132 | NM_020412.4 | NP_065145.2 | Vps46B; C10orf2; |
| | | | | C18orf2; CHMP1.5; |
| | | | | Vps46-2; C18- |
| | | | | ORF2 |
| RFWD3 | 55159 | NM_018124.3 | NP_060594.3 | RNF201; FLJ10520 |
| XRCC6 | 2547 | NM_001469.3 | NP_001460.1 | ML8; KU70; |
| | | | | TLAA; CTC75; |
| | | | | CTCBF; G22P1 |
| UGDH | 7358 | NM_003359.2 | NP_003350.1 | GDH; UGD; |
| | | | | UDPGDH; UDP- |
| | | | | GlcDH |
| RPF2P | 729608 | | | BXDC1P; C20orf53; |
| | | | | bA353C18.4 |

As used herein, the term “nucleic acid” refers to single or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the moieties discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides), a reduction in the AT content of AT rich regions, or replacement of non-preferred codon usage of the expression system to preferred codon usage of the expression system. The nucleic acid can be directly cloned into an appropriate vector, or if desired, can be modified to facilitate the subsequent cloning steps. Such modification steps are routine, an example of which is the addition of oligonucleotide linkers which contain restriction sites to the termini of the nucleic acid. General methods are set forth in in Sambrook et al. (2001) Molecular Cloning—A Laboratory Manual (3rd ed.) Vol. 1-3, Cold Spring Harbor Laboratory, Cold Spring Harbor Press, NY, (Sambrook).

Once the nucleic acid sequence is obtained, the sequence encoding the specific amino acids can be modified or changed at any particular amino acid position by techniques well known in the art. For example, PCR primers can be designed which span the amino acid position or positions and which can substitute any amino acid for another amino acid. Alternatively, one skilled in the art can introduce specific mutations at any point in a particular nucleic acid sequence through techniques for point mutagenesis. General methods are set forth in Smith, M. “In vitro mutagenesis” Ann. Rev. Gen., 19:423-462 (1985) and Zoller, M. J. “New molecular biology methods for protein engineering” Curr. Opin. Struct. Biol., 1:605-610 (1991), which are incorporated herein in their entirety for the methods. These techniques can be used to alter the coding sequence without altering the amino acid sequence that is encoded.

The sequences contemplated herein include full-length wild-type (or native) sequences, as well as allelic variants, variants, fragments, homologs or fusion sequences that retain the ability to function as the cellular nucleic acid or protein involved in viral infection. In certain examples, a protein or nucleic acid sequence has at least 50% sequence identity, for example at least 50%, 55%, 60%, 65%, 70%, 75%, 80%, 85%, 90%, 95%, or 98% sequence identity to a native sequences of the genes set forth in Table 1. In other examples, a nucleic acid sequence involved in viral infection has a sequence that hybridizes to a sequence of a gene set forth in Table 1 and retains the activity of the sequence of the gene set forth in Table 1. For example, and not to be limiting, a nucleic acid that hybridizes to an AHR nucleic acid sequence and encodes a protein that retains AHR activity is contemplated by the present invention. Such sequences include the genomic sequence for the genes set forth in Table 1. The examples set forth above for AHR are merely illustrative and should not be limited to AHR as the analysis set forth in this example applies to every nucleic acid and protein for the genes listed in Table 1.

Unless otherwise specified, any reference to a nucleic acid molecule includes the reverse complement of the nucleic acid. Except where single-strandedness is required by the text herein (for example, a ssRNA molecule), any nucleic acid written to depict only a single strand encompasses both strands of a corresponding double-stranded nucleic acid. Fragments of the nucleic acids for the genes set forth in Table 1 and throughout the specification are also contemplated. These fragments can be utilized as primers and probes to amplify, inhibit or detect any of the nucleic acids or genes set forth in Table 1.

Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (Detects Sequences that Share 90% Identity)
Hybridization: 5×SSC at 65° C. for 16 hours
Wash twice: 2×SSC at room temperature (RT) for 15 minutes each
Wash twice: 0.5×SSC at 65° C. for 20 minutes each
High Stringency (Detects Sequences that Share 80% Identity or Greater)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
Low Stringency (Detects Sequences that Share Greater than 50% Identity)
Hybridization: 6×SSC at RT to 55° C. for 16-20 hours
Wash at least twice: 2×-3×SSC at RT to 55° C. for 20-30 minutes each.

Also provided is a vector, comprising a nucleic acid set forth herein. The vector can direct the in vivo or in vitro synthesis of any of the proteins or polypeptides described herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene (See generally, Sambrook et al.). The vector, for example, can be a plasmid. The vectors can contain genes conferring hygromycin resistance, ampicillin resistance, gentamicin resistance, neomycin resistance or other genes or phenotypes suitable for use as selectable markers, or methotrexate resistance for gene amplification.

There are numerous other E. coli (Escherichia coli) expression vectors, known to one of ordinary skill in the art, which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. Additionally, yeast expression can be used. The invention provides a nucleic acid encoding a polypeptide of the present invention, wherein the nucleic acid can be expressed by a yeast cell. More specifically, the nucleic acid can be expressed by Pichia pastoris or S. cerevisiae.

Mammalian cells also permit the expression of proteins in an environment that favors important post-translational modifications such as folding and cysteine pairing, addition of complex carbohydrate structures, and secretion of active protein. Vectors useful for the expression of active proteins are known in the art and can contain genes conferring hygromycin resistance, genticin or G418 resistance, or other genes or phenotypes suitable for use as selectable markers, or methotrexate resistance for gene amplification. A number of suitable host cell lines capable of secreting intact human proteins have been developed in the art, and include the CHO cell lines, HeLa cells, COS-7 cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc.

The expression vectors described herein can also include nucleic acids of the present invention under the control of an inducible promoter such as the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the art are also contemplated. Furthermore, a Cre-loxP inducible system can also be used, as well as a Flp recombinase inducible promoter system, both of which are known in the art.

Insect cells also permit the expression of mammalian proteins. Recombinant proteins produced in insect cells with baculovirus vectors undergo post-translational modifications similar to that of wild-type proteins. The invention also provides for the vectors containing the contemplated nucleic acids in a host suitable for expressing the nucleic acids. The host cell can be a prokaryotic cell, including, for example, a bacterial cell. More particularly, the bacterial cell can be an E. coli cell. Alternatively, the cell can be a eukaryotic cell, including, for example, a Chinese hamster ovary (CHO) cell, a COS-7 cell, a HELA cell, an avian cell, a myeloma cell, a Pichia cell, or an insect cell. A number of other suitable host cell lines have been developed and include myeloma cell lines, fibroblast cell lines, a cell line suitable for infection by a pathogen, and a variety of tumor cell lines such as melanoma cell lines. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, Lipofectamine, or lipofectin mediated transfection, electroporation or any method now known or identified in the future can be used for other eukaryotic cellular hosts.

### Polypeptides

The present invention provides isolated polypeptides comprising the polypeptide or protein sequences for the genes set forth in Table 1. The present invention also provides fragments of these polypeptides. These fragments can be of sufficient length to serve as antigenic peptides for the generation of antibodies. The present invention also contemplates functional fragments that possess at least one activity of a gene or gene product listed in Table 1, for example, involved in viral infection.

By “isolated polypeptide” or “purified polypeptide” is meant a polypeptide that is substantially free from the materials with which the polypeptide is normally associated in nature or in culture. The polypeptides of the invention can be obtained, for example, by extraction from a natural source if available (for example, a mammalian cell), by expression of a recombinant nucleic acid encoding the polypeptide (for example, in a cell or in a cell-free translation system), or by chemically synthesizing the polypeptide. In addition, a polypeptide can be obtained by cleaving full length polypeptides. When the polypeptide is a fragment of a larger naturally occurring polypeptide, the isolated polypeptide is shorter than and excludes the full-length, naturally-occurring polypeptide of which it is a fragment.

Also provided by the present invention is a polypeptide comprising an amino acid sequence at least about 50%, 55%, 60%, 65%, 70%, 75%, 80%, 85%, 90%, 95%, 96%, 97%, 98% or 99% identical to the native polypeptide sequence for any gene set forth in Table 1. It is understood that as discussed herein the use of the terms “homology” and “identity” mean the same thing as similarity. Thus, for example, if the use of the word homology is used to refer to two non-natural sequences, it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related.

In general, it is understood that one way to define any known variants and derivatives or those that might arise, of the disclosed nucleic acids and polypeptides herein, is through defining the variants and derivatives in terms of homology to specific known sequences. In general, variants of nucleic acids and polypeptides herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two polypeptides or nucleic acids.

### Methods of Decreasing Infection

The present invention provides a method of inhibiting infection in a cell by a pathogen comprising decreasing expression or activity of a gene or gene product set forth in Table 1. As stated above, an infection can be a viral infection, bacterial infection, fungal infection or a parasitic infection, to name a few. A decrease or inhibition of infection can occur in a cell, in vitro, ex vivo or in vivo. As utilized throughout, the term “infection” encompasses all phases of pathogenic life cycles including, but not limited to, attachment to cellular receptors, entry, internalization, disassembly, replication, genomic integration of pathogenic sequences, transcription of viral RNA, translation of viral RNA, transcription of host cell mRNA, translation of host cell mRNA, proteolytic cleavage of pathogenic proteins or cellular proteins, assembly of particles, endocytosis, cell lysis, budding, and egress of the pathogen from the cells. Therefore, a decrease in infection can be a decrease in attachment to cellular receptors, a decrease in entry, a decrease in internalization, a decrease in disassembly, a decrease in replication, a decrease in genomic integration of pathogenic sequences, a decrease in translation of mRNA, a decrease in proteolytic cleavage of pathogenic proteins or cellular proteins, a decrease in assembly of particles, a decrease in endocytosis, a decrease in cell lysis, a decrease in budding, or a decrease in egress of the pathogen from the cells. This decrease does not have to be complete as this can range from a slight decrease to complete ablation of the infection. A decrease in infection can be at least about 10%, 20%, 30%, 40%, 50%, 60, 70%, 80%, 90%, 95%, 100% or any other percentage decrease in between these percentages as compared to the level of infection in a cell wherein expression or activity of a gene or gene product set forth in Table 1 has not been decreased.

In the methods set forth herein, expression can be inhibited, for example, by inhibiting transcription of the gene, or inhibiting translation of its gene product. Similarly, the activity of a gene product (for example, an mRNA, a polypeptide or a protein) can be inhibited, either directly or indirectly. Inhibition or a decrease in expression does not have to be complete as this can range from a slight decrease in expression to complete ablation of expression. For example, expression can be inhibited by about 10%, 20%, 30%, 40%, 50%, 60%, 70%, 80%, 90%, 95%, 99%, 100% or any percentage in between as compared to a control cell wherein the expression of a gene or gene product set forth in Table 1 has not been decreased or inhibited. Similarly, inhibition or decrease in the activity of a gene product does not have to be complete as this can range from a slight decrease to complete ablation of the activity of the gene product. For example, the activity of a gene product can be inhibited by about 10%, 20%, 30%, 40%, 50%, 60%, 70%, 80%, 90%, 95%, 99%, 100% or any percentage in between as compared to a control cell wherein activity of a gene or gene product set forth in Table 1 has not been decreased or inhibited. As utilized herein, “activity of a gene product” can be an activity that is involved in pathogenicity, for example, interacting directly or indirectly, with pathogen, e.g. viral protein or viral nucleic acids, or an activity that the gene product performs in a normal cell, i.e. in a non-infected cell. Depending on the gene product, one of skill in the art would know how to assay for an activity that is involved in pathogenicity, an activity that is involved in normal cellular function, or both. As set forth above, an activity of the proteins and nucleic acids listed herein can be the ability to bind or interact with other proteins. Therefore, the present invention also provides a method of decreasing infection by inhibiting or decreasing the interaction between any of the proteins of the present invention and other cellular proteins, such as, for example, receptors, enzymes, nucleic acids and hormones, provided that such inhibition correlates with decreasing infection by the pathogen. Also provided is a method of decreasing infection by inhibiting or decreasing the interaction between any of the proteins of the present invention and a viral, bacterial, parasitic or fungal protein (i.e. a non-host protein).

The cells of the present invention can be prokaryotic or eukaryotic, such as a cell from an insect, fish, crustacean, mammal, bird, reptile, yeast or a bacterium, such as E. coli. The cell can be part of an organism, or part of a cell culture, such as a culture of mammalian cells or a bacterial culture. Therefore, the cell can also be part of a population of cells. The cell(s) can also be in a subject.

Examples of viral infections include but are not limited to, infections caused by RNA viruses (including negative stranded RNA viruses, positive stranded RNA viruses, double stranded RNA viruses and retroviruses), or DNA viruses. All strains, types, and subtypes of RNA viruses and DNA viruses are contemplated herein.

Examples of RNA viruses include, but are not limited to picornaviruses, which include aphthoviruses (for example, foot and mouth disease virus 0, A, C, Asia 1, SAT1, SAT2 and SAT3), cardioviruses (for example, encephalomycarditis virus and Theiller's murine encephalomyelitis virus), enteroviruses (for example polioviruses 1, 2 and 3, human enteroviruses A-D, bovine enteroviruses 1 and 2, human coxsackieviruses A1-A22 and A24, human coxsackieviruses B1-B5, human echoviruses 1-7, 9, 11-12, 24, 27, 29-33, human enteroviruses 68-71, porcine enteroviruses 8-10 and simian enteroviruses 1-18), erboviruses (for example, equine rhinitis virus), hepatovirus (for example human hepatitis A virus and simian hepatitis A virus), kobuviruses (for example, bovine kobuvirus and Aichi virus), parechoviruses (for example, human parechovirus 1 and human parechovirus 2), rhinovirus (for example, rhinovirus A, rhinovirus B, rhinovirus C, HRV₁₆, HRV₁₆ (VR-11757), HRV₁₄ (VR-284), or HRV_{1A} (VR-1559), human rhinovirus 1-100 and bovine rhinoviruses 1-3) and teschoviruses (for example, porcine teschovirus).

Additional examples of RNA viruses include caliciviruses, which include noroviruses (for example, Norwalk virus), sapoviruses (for example, Sapporo virus), lagoviruses (for example, rabbit hemorrhagic disease virus and European brown hare syndrome) and vesiviruses (for example vesicular exanthema of swine virus and feline calicivirus).

Other RNA viruses include astroviruses, which include mamastorviruses and avastroviruses. Togaviruses are also RNA viruses. Togaviruses include alphaviruses (for example, Chikungunya virus, Sindbis virus, Semliki Forest virus, Western equine encephalitis, Getah virus, Everglades virus, Venezuelan equine encephalitis virus and Aura virus) and rubella viruses. Additional examples of RNA viruses include the flaviviruses (for example, tick-borne encephalitis virus, Tyuleniy virus, Aroa virus, Dengue virus (types 1 to 4), Kedougou virus, Japanese encephalitis virus (JEV), West Nile virus (WNV), Kokobera virus, Ntaya virus, Spondweni virus, Yellow fever virus, Entebbe bat virus, Modoc virus, R10 Bravo virus, Cell fusing agent virus, pestivirus, GB virus A, GBV-A like viruses, GB virus C, Hepatitis G virus, hepacivirus (hepatitis C virus (HCV)) all six genotypes), bovine viral diarrhea virus (BVDV) types 1 and 2, and GB virus B).

Other examples of RNA viruses are the coronaviruses, which include, human respiratory coronaviruses such as SARS-CoV, HCoV-229E, HCoV-NL63 and HCoV-OC43. Coronaviruses also include bat SARS-like CoV, turkey coronavirus, chicken coronavirus, feline coronavirus and canine coronavirus. Additional RNA viruses include arteriviruses (for example, equine arterivirus, porcine reproductive and respiratory syndrome virus, lactate dehyrogenase elevating virus of mice and simian hemorraghic fever virus). Other RNA viruses include the rhabdoviruses, which include lyssaviruses (for example, rabies, Lagos bat virus, Mokola virus, Duvenhage virus and European bat lyssavirus), vesiculoviruses (for example, VSV-Indiana, VSV-New Jersey, VSV-Alagoas, Piry virus, Cocal virus, Maraba virus, Isfahan virus and Chandipura virus), and ephemeroviruses (for example, bovine ephemeral fever virus, Adelaide River virus and Berrimah virus). Additional examples of RNA viruses include the filoviruses. These include the Marburg and Ebola viruses (for example, EBOV-Z, EBOV-S, EBOV-IC and EBOV-R.

The paramyxoviruses are also RNA viruses. Examples of these viruses are the rubulaviruses (for example, mumps, parainfluenza virus 5, human parainfluenza virus type 2, Mapuera virus and porcine rubulavirus), avulaviruses (for example, Newcastle disease virus), respoviruses (for example, Sendai virus, human parainfluenza virus type 1 and type 3, bovine parainfluenza virus type 3), henipaviruses (for example, Hendra virus and Nipah virus), morbilloviruses (for example, measles, Cetacean morvilliirus, Canine distemper virus, Peste-des-petits-ruminants virus, Phocine distemper virus and Rinderpest virus), pneumoviruses (for example, human respiratory syncytial virus A2, B1 and S2, bovine respiratory syncytial virus and pneumonia virus of mice), metapneumoviruses (for example, human metapneumovirus and avian metapneumovirus). Additional paramyxoviruses include Fer-de-Lance virus, Tupaia paramyxovirus, Menangle virus, Tioman virus, Beilong virus, J virus, Mossman virus, Salem virus and Nariva virus.

Additional RNA viruses include the orthomyxoviruses. These viruses include influenza viruses and strains (e.g., influenza A, influenza A strain A/Victoria/3/75, influenza A strain A/Puerto Rico/8/34, influenza A H1N1 (including but not limited to A/WS/33, A/NWS/33 and A/California/04/2009 strains), influenza B, influenza B strain Lee, and influenza C viruses) H2N2, H3N2, H5N1, H7N7, H1N2, H9N2, H7N2, H7N3 and H10N7), as well as avian influenza (for example, strains H5N1, H5N1 Duck/MN/1525/81, H5N2, H7N1, H7N7 and H9N2) thogotoviruses and isaviruses. Orthobunyaviruses (for example, Akabane virus, California encephalitis, Cache Valley virus, Snowshoe hare virus) nairoviruses (for example, Nairobi sheep virus, Crimean-Congo hemorrhagic fever virus Group and Hughes virus), phleboviruses (for example, Candiru, Punta Toro, Rift Valley Fever, Sandfly Fever, Naples, Toscana, Sicilian and Chagres), and hantaviruses (for example, Hantaan, Dobrava, Seoul, Puumala, Sin Nombre, Bayou, Black Creek Canal, Andes and Thottapalayam) are also RNA viruses. Arenaviruses such as lymphocytic choriomeningitis virus, Lujo virus, Lassa fever virus, Argentine hemorrhagic fever virus, Bolivian hemorrhagic fever virus, Venezuelan hemorrhagic fever virus, SABV and WWAV are also RNA viruses. Borna disease virus is also an RNA virus. Hepatitis D (Delta) virus and hepatitis E are also RNA viruses.

Additional RNA viruses include reoviruses, rotaviruses, birnaviruses, chrysoviruses, cystoviruses, hypoviruses partitiviruses and totoviruses. Orbiviruses such as African horse sickness virus, Blue tongue virus, Changuinola virus, Chenuda virus, Chobar Gorge Corriparta virus, epizootic hemorraghic disease virus, equine encephalosis virus, Eubenangee virus, Ieri virus, Great Island virus, Lebombo virus, Orungo virus, Palyam virus, Peruvian Horse Sickness virus, St. Croix River virus, Umatilla virus, Wad Medani virus, Wallal virus, Warrego virus and Wongorr virus are also RNA viruses.

Retroviruses include alpharetroviruses (for example, Rous sarcoma virus and avian leukemia virus), betaretroviruses (for example, mouse mammary tumor virus, Mason-Pfizer monkey virus and Jaagsiekte sheep retrovirus), gammaretroviruses (for example, murine leukemia virus and feline leukemia virus, deltraretroviruses (for example, human T cell leukemia viruses (HTLV-1, HTLV-2), bovine leukemia virus, STLV-1 and STLV-2), epsilonretriviruses (for example, Walleye dermal sarcoma virus and Walleye epidermal hyperplasia virus 1), reticuloendotheliosis virus (for example, chicken syncytial virus, lentiviruses (for example, human immunodeficiency virus (HIV) type 1, human immunodeficiency virus (HIV) type 2, human immunodeficiency virus (HIV) type 3, simian immunodeficiency virus, equine infectious anemia virus, feline immunodeficiency virus, caprine arthritis encephalitis virus and Visna maedi virus) and spumaviruses (for example, human foamy virus and feline syncytia-forming virus).

Examples of DNA viruses include polyomaviruses (for example, simian virus 40, simian agent 12, BK virus, JC virus, Merkel Cell polyoma virus, bovine polyoma virus and lymphotrophic papovavirus), papillomaviruses (for example, human papillomavirus, bovine papillomavirus, adenoviruses (for example, adenoviruses A-F, canine adenovirus type I, canined adeovirus type 2), circoviruses (for example, porcine circovirus and beak and feather disease virus (BFDV)), parvoviruses (for example, canine parvovirus), erythroviruses (for example, adeno-associated virus types 1-8), betaparvoviruses, amdoviruses, densoviruses, iteraviruses, brevidensoviruses, pefudensoviruses, herpes viruses 1, 2, 3, 4, 5, 6, 7 and 8 (for example, herpes simplex virus 1, herpes simplex virus 2, varicella-zoster virus, Epstein-Barr virus, cytomegalovirus, Kaposi's sarcoma associated herpes virus, human herpes virus-6 variant A, human herpes virus-6 variant B and cercophithecine herpes virus 1 (B virus)), poxviruses (for example, smallpox (variola), cowpox, monkeypox, vaccinia, Uasin Gishu, camelpox, psuedocowpox, pigeonpox, horsepox, fowlpox, turkeypox and swinepox), and hepadnaviruses (for example, hepatitis B and hepatitis B-like viruses). Chimeric viruses comprising portions of more than one viral genome are also contemplated herein.

For animals, in addition to the animal viruses listed above, viruses include, but are not limited to, the animal counterpart to any above listed human virus. The provided compounds can also decrease infection by newly discovered or emerging viruses. Such viruses are continuously updated on http://en.wikipedia.org/wiki/Virus and www.virology.net.

Examples of bacterial infections include, but are not limited to infections caused by the following bacteria: Listeria (sp.), Franscicella tularensis, Mycobacterium tuberculosis, Rickettsia (all types), Ehrlichia, Chlamydia. Further examples of bacteria that can be targeted by the present methods include M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Kingella kingae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Examples of parasitic infections include, but are not limited to infections caused by the following parasites: Cryptosporidium, Plasmodium (all species), American trypanosomes (T. cruzi), African trypanosomes, Acanthamoeba, Entaoeba histolytica, Angiostrongylus, Anisakis, Ascaris, Babesia, Balantidium, Baylisascaris, lice, ticks, mites, fleas, Capillaria, Clonorchis, Chilomastix mesnili, Cyclspora, Diphyllobothrium, Dipylidium caninum, Fasciola, Giardia, Gnathostoma, Hetetophyes, Hymenolepsis, Isospora, Loa loa, Microsporidia, Naegleria, Toxocara, Onchocerca, Opisthorchis, Paragonimus, Baylisascaris, Strongyloides, Taenia, Trichomonas and Trichuris.

Furthermore, examples of protozoan and fungal species contemplated within the present methods include, but are not limited to, Plasmodium falciparum, other Plasmodium species, Toxoplasma gondii, Pneumocystis carinii, Trypanosoma cruzi, other trypanosomal species, Leishmania donovani, other Leishmania species, Theileria annulata, other Theileria species, Eimeria tenella, other Eimeria species, Histoplasma capsulatum, Cryptococcus neoformans, Blastomyces dermatitidis, Coccidioides immitis, Paracoccidioides brasiliensis, Penicillium marneffei, and Candida species. The provided compounds can also decrease infection by newly discovered or emerging bacteria, parasites or fungi, including multidrug resistant strains of same.

Further provided by the present invention is a method of inhibiting infection in a cell by a pathogen comprising decreasing expression or activity of a gene or gene product set forth in Table 1, wherein the pathogen is a respiratory virus. Respiratory viruses include, but are not limited to, picornaviruses, orthomyxoviruses, paramyxoviruses, coronaviruses and adenoviruses. More specifically, and not to be limiting, the respiratory virus can be an influenza virus, a parainfluenza virus, an adenovirus, a rhinovirus or a respiratory syncytial virus (RSV).

Also provided by the present invention is a method of inhibiting infection in a cell by a pathogen comprising decreasing expression or activity of a gene or gene product set forth in Table 1, wherein the pathogen is a gastrointestinal virus. Gastrointestinal viruses include, but are not limited to, picornaviruses, filoviruses, flaviviruses, calciviruses and reoviruses. More specifically, and not to be limiting, the gastrointestinal virus can be a reovirus, a Norwalk virus, an Ebola virus, a Marburg virus, a rotavirus, an enterovirus, a Dengue fever virus, a yellow fever virus, or a West Nile virus.

The present invention also provides a method of inhibiting infection in a cell by a pathogen comprising decreasing expression or activity of a gene or gene product set forth in Table 1, wherein the pathogen is a pox virus, BVDV, a herpes virus, HIV, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

Also provided is a method of inhibiting infection in a cell by a pathogen comprising decreasing expression or activity of a gene or gene product set forth in Table 1, wherein the pathogen is a pox virus, LCM, Junin virus, Machupo virus, Guanarito virus, Lassa Fever virus, hantavirus, Rift Valley Fever virus Ebola virus, Marburg virus or Dengue Fever virus.

The present invention also provides a method of decreasing the toxicity of a toxin in a cell comprising decreasing expression or activity of a gene or gene product set forth in Table 1. The cell can be in vitro, ex vivo or in vivo. Toxins can include, but are not limited to, a bacterial toxin, neurotoxins, such as botulinum neurotoxins, mycotoxins, ricin, Clostridium perfringens toxins, Clostridium difficile toxins, saxitoxins, tetrodotoxins, abrin, conotoxins, Staphlococcal toxins, E. coli toxins, streptococcal toxins, shigatoxins, T-2 toxins, anthrax toxins, chimeric forms of the toxins listed herein, and the like. The decrease in toxicity can be at least about 10%, 20%, 30%, 40%, 50%, 60, 70%, 80%, 90%, 95%, 100% or any other percentage decrease in between these percentages as compared to the level of toxicity in a cell wherein expression or activity of a gene or gene product set forth in Table 1 has not been decreased.

Toxicity can be measured, for example, via a cell viability, apopotosis assay, LDH release assay or cytotoxicity assay (See, for example, Kehl-Fie and St. Geme “Identification and characterization of an RTX toxin in the emerging pathogen Kingella kingae,” J. Bacteriol. 189(2):430-6 (2006) and Kirby “Anthrax Lethal Toxin Induces Human Endothelial cell Apoptosis,” Infection and Immunity 72: 430-439 (2004), both of which are incorporated herein in their entireties by this reference.)

In the methods of the present invention, expression and/or activity of a gene or gene product set forth in Table 1 can be decreased by contacting the cell with any composition that can decrease expression or activity. For example, the composition can comprise a chemical, a small or large molecule (organic or inorganic), a drug, a protein, a peptide, a cDNA, an antibody, a morpholino, a triple helix molecule, an aptamer, an siRNA, a shRNA, an miRNA, an antisense RNA, a ribozyme or any other compound now known or identified in the future that decreases the expression and/or activity of a gene or gene product set forth in Table 1. A decrease in expression or activity can occur by decreasing transcription of mRNA or decreasing translation of RNA. A composition can also be a mixture or “cocktail” of two or more of the compositions described herein.

These compositions can be used alone or in combination with other therapeutic agents such as antiviral compounds, antibacterial agents, antifungal agents, antiparasitic agents, anti-inflammatory agents, anti-cancer agents, etc. All of the compounds described herein can be contacted with a cell in vitro, ex vivo or in vivo.

Examples of antiviral compounds include, but are not limited to, amantadine, rimantadine, ribavirin, zanamavir (Relenza®) and oseltamavir (Tamiflu®) for the treatment of flu and its associated symptoms. Antiviral compounds useful in the treatment of HIV include Combivir® (lamivudine-zidovudine), maraviroc, Crixivan® (indinavir), Emtriva® (emtricitabine), Epivir® (lamivudine), Fortovase® (saquinavir-sg), Hivid® (zalcitabine), Invirase® (saquinavir-hg), Kaletra® (lopinavir-ritonavir), Lexiva™ (fosamprenavir), Norvir® (ritonavir), Retrovir® (zidovudine), Sustiva® (efavirenz), Videx EC® (didanosine), Videx® (didanosine), Viracept® (nelfinavir), Viramune® (nevirapine), Zerit® (stavudine), Ziagen® (abacavir), Fuzeon® (enfuvirtide), Rescriptor® (delavirdine), Reyataz®(atazanavir), Trizivir® (abacavir-lamivudine-zidovudine), Viread® (tenofovir disoproxil fumarate), Truvada® (tenofovir-emtricitabine), Atripla® (tenofovir-emtricitabine-efavirenz) and Agenerase® (amprenavir). Other antiviral compounds useful in the treatment of Ebola and other filoviruses include ribavirin and cyanovirin-N (CV-N). For the treatment of herpes virus, Zovirax® (acyclovir) is available. Antibacterial agents include, but are not limited to, antibiotics (for example, penicillin and ampicillin), sulfa Drugs and folic acid Analogs, Beta-Lactams, aminoglycosides, tetracyclines, macrolides, lincosamides, streptogramins, fluoroquinolones, rifampin, mupirocin, cycloserine, aminocyclitol and oxazolidinones.

Antifungal agents include, but are not limited to, amphotericin, nystatin, terbinafine, itraconazole, fluconazole, ketoconazole, and griselfulvin.

Antiparasitic agents include, but are not limited to, antihelmintics, antinematodal agents, antiplatyhelmintic agents, antiprotozoal agents, amebicides, antimalarials, antitrichomonal agents, aoccidiostats and trypanocidal agents.

### Antibodies

The present invention also provides antibodies that specifically bind to the gene products, proteins and fragments thereof set forth in Table 1. The antibody of the present invention can be a polyclonal antibody or a monoclonal antibody. The antibody of the invention selectively binds a polypeptide. By “selectively binds” or “specifically binds” is meant an antibody binding reaction which is determinative of the presence of the antigen (in the present case, a polypeptide set forth in Table 1 or antigenic fragment thereof among a heterogeneous population of proteins and other biologics). Thus, under designated immunoassay conditions, the specified antibodies bind preferentially to a particular peptide and do not bind in a significant amount to other proteins in the sample. Preferably, selective binding includes binding at about or above 1.5 times assay background and the absence of significant binding is less than 1.5 times assay background.

This invention also contemplates antibodies that compete for binding to natural interactors or ligands to the proteins set forth in Table 1. In other words, the present invention provides antibodies that disrupt interactions between the proteins set forth in Table 1 and their binding partners. For example, an antibody of the present invention can compete with a protein for a binding site (e.g. a receptor) on a cell or the antibody can compete with a protein for binding to another protein or biological molecule, such as a nucleic acid that is under the transcriptional control of a transcription factor set forth in Table 1. An antibody can also disrupt the interaction between a protein set forth in Table 1 and a pathogen, or the product of a pathogen. For example, an antibody can disrupt the interaction between a protein set forth in Table 1 and a viral protein, a bacterial protein, a parasitic protein, a fungal protein or a toxin. The antibody optionally can have either an antagonistic or agonistic function as compared to the antigen. Antibodies which antagonize pathogenic infection are utilized to decrease infection.

Preferably, the antibody binds a polypeptide in vitro, ex vivo or in vivo. Optionally, the antibody of the invention is labeled with a detectable moiety. For example, the detectable moiety can be selected from the group consisting of a fluorescent moiety, an enzyme-linked moiety, a biotin moiety and a radiolabeled moiety. The antibody can be used in techniques or procedures such as diagnostics, screening, or imaging. Anti-idiotypic antibodies and affinity matured antibodies are also considered to be part of the invention.

As used herein, the term “antibody” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. In one embodiment of the invention, the “humanized” antibody is a human version of the antibody produced by a germ line mutant animal. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat or rabbit having the desired specificity, affinity and capacity. In one embodiment, the present invention provides a humanized version of an antibody, comprising at least one, two, three, four, or up to all CDRs of a monoclonal antibody that specifically binds to a protein or fragment thereof set forth in Table 1. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Examples of antibodies corresponding to those genes found in Table 1 are provided below in Table 2. This is in no way limiting, as one of skill in the art can readily identify and create other antibodies corresponding to any of the genes in Table 1.

TABLE 2

| NAME | Antibodies |
|---|---|
| PCBP1 | H00005093-M01 (Novus) |
| AREGB | sc-5794 (Santa Cruz Biotech) |
| UBXN6 | H00051035-B01 (Novus) |
| CUGBP1 | NBP1-19606 (Novus) |
| MATR3 | PA1-27909 (Thermo Scientific, Pierce Antibodies) |
| TCF25 | LS-C81094 (Lifespan Biosciences, Inc.) |
| TOB2 | H00010766-M01 (Novus) |
| ECT2 | NBP1-30872 (Novus) |
| GPR113 | sc-137507 (Santa Cruz Biotech) |
| SEL1 | MBS420228 (My Biosource LLC) |
| PAIP2 | sc-365317 (Santa Cruz Biotech) |
| PHF15 | H00023338-B02 (Novus) |
| GSTCD | H00079807-B01P (Novus) |
| INTS12 | sc-130155 (Santa Cruz Biotech) |
| RPS12 | H00006183-B01 (Novus) |
| RPL35A | H00006165-A01 (Novus) |
| KRT86 | LS-C20848 (Lifespan Biosci) |
| WTAP | |
| SFRS11 | NB100-68245 (Novus) |
| ALS2 | sc-160091 (Santa Cruz Biotech) |
| DNAJB9 | H00057679-M01 (Novus) |
| THAP5 | NBP1-32249 (Novus) |
| ARF6 | sc-138685 (Santa Cruz Biotech) |
| BAX | H00000382-M01A (Novus) |
| FTL | sc-20068 (Santa Cruz Biotech) |
| HSPA8 | MBS619637 (My Biosource, LLC) |
| KIF12 | orb19293 (Biorbyt) |
| RAB1A | H00113220-B01 (Novus) |
| SCTR | MBS301542 (My Biosource) |
| TFPI | H00006344-B01 (Novus) |
| ZNF827 | sc-81744 (Santa Cruz Biotech) |
| DUSP16 | LS-C107545 (LifeSpan Biosciences) |
| RNLS | NB100-848 (Novus) |
| ARF4 | TA307077 (Origene) |
| FOSL2 | H00000379-B01 (Novus) |
| GDE1 | LS-C66280 (LifeSpan Biosci) |
| ZNF581 | sc-133615 (Santa Cruz Biotech) |
| TRIB1 | PA1-27759 (Thermo Scientific Pierce Antibodies) |
| NFE2L1 | NB600-1452 (Novus Biologicals) |
| RPL22L1 | LS-C110256 |
| RAB9B | sc-100126 (Santa Cruz Biotech) |
| SYNE2 | NBP1-03411 (Novus) |
| HNF1B | sc-365431 (Santa Cruz Biotech) |
| ALDOA | sc-7411 (Santa Cruz Biotech) |
| CDKN1B | sc-53933 (Santa Cruz Biotech) |
| TBCK | PA1-38350 (Thermo Scientific) |
| PARD6B | H00093627-M01 (Novus Biologicals) |
| RSPRY1 | sc-133205 (Santa Cruz Biotech) |
| BANF1 | H00089970-B01 (Novus) |
| EIF1AD | NBP1-02976 (Novus) |
| ARPC5L | sc-242607 (Santa Cruz) |
| PAFAH1B1 | A0760-89 (US Biological) |
| BAT2L | A0760-89 (US Biological) |
| TOR1AIP2 | sc-169981 (Santa Cruz Biotech) |
| SON | H00002618-M01 (Novus) |
| STK35 | 46460002 (Novus) |
| RBM5 | 46460002 (Novus) |
| HNRNPUL2 | NBP1-26613 (Novus) |
| TTC9C | LS-C120038 (Lifespan Biosci) |
| RAB5C | sc-138714 (Santa Cruz Biotech) |
| EIF4B | NBP1-03409 (Novus) |
| SFXN2 | TA303495 (Origene Tech) |
| ARL3 | H00118980-B01P (Novus) |
| FTSJ1 | H00000403-B01 (Novus) |
| TGIF1 | H00024140-B01 (Novus) |
| RPL35 | TA300819 (Origene) |
| RPL7 | H00011224-A01 (Novus) |
| PDE9A | NB100-2269 (Novus) |
| PKHD1L1 | sc-271754 (Santa Cruz Biotech) |
| BOLA1 | H00093035-M01 (Novus) |
| AGAP1 | H00051027-M01 (Novus) |
| STAU2 | MBS615380 (My Biosource) |
| HSP90AB1 | H00027067-M14 (Novus) |
| PCID2 | TA500494 (Origene) |
| RPL26 | H00055795-B01P (Novus) |
| CTNND1 | H00051121-A01 (Novus) |
| SF3A2 | TA303472 (Origene) |
| PLEKHJ1 | H00008175-M01 (Novus) |
| PPP1R9A | MBS420661 (My BioSource) |
| HIPK3 | LS-C73316 (LifeSpan BioSci) |
| ASB6 | NBP1-31827 (Novus) |
| SENP3 | H00140459-B01P (Novus) |
| TNFSF12 | NB100-92103 (Novus) |
| TNFSF13 | TA306313 (Origene) |
| PANK1 | NB100-94349 (Novus) |
| ACTN4 | NBP1-32509 (Novus) |
| NUMA1 | CB1024 (EMD Millipore) |
| TBL1XR1 | TA307173 (Origene) |
| PA2G4 | sc-100908 (Santa Cruz) |
| FLRT3 | AF3189 (R&D Systems) |
| SLC25A37 | BAF2795 (R&D Systems) |
| RPS8 | LS-C51646 (LS Bio) |
| RBM3 | YSRTAHP1543 (Accurate Chemical) |
| RANGAP1 | NBP1-36980 (Novus) |
| BMPR2 | MBS420913 (MyBioSource) |
| DHX9 | orb13108 (BioOrbyt) |
| SREBF2 | orb20223 (BioOrbyt) |
| MARCH7 | sc-5603 (Santa Cruz) |
| RPL29 | YSRTAHP1543 (Accurate) |
| CSRP1 | H00006159-A01 (Novus) |
| AP1G1 | NBP1-41448 (Novus) |
| AHCY | MBS611453 (MyBioSource) |
| CDH6 | A1059-73E (US Biological) |
| PABPN1 | sc-31024 (Santa Cruz Biotech) |
| NPAS2 | NBP1-31805 (Novus) |
| APBB1IP | H00004862-M03 (Novus) |
| SETD5 | TA307075 (Origene) |
| PARC | 22560002 (Novus) |
| DR1 | LS-C89778 (LS BIO) |
| LEPREL1 | NBP1-03329 (Novus) |
| DIAPH3 | sc-102652 (Santa Cruz Biotech) |
| CPS1 | MBS421252 (My BioSource) |
| PKM2 | NBP1-49217 (Novus) |
| SEMA3F | NBP1-49217 (My Biosource) |
| MANBAL | S3205 (Epitomics) |
| LIMCH1 | sc-86167 (Santa Cruz) |
| PFAS | NBP1-32614 (Novus) |
| CSGALNACT2 | LS-C80851 (LifeSpanBio) |
| FLVCR2 | H00055454-D01P (Novus) |
| HSPE1 | (My BioSource) |
| RPL27 | YSRTAHP1543 (Accurate Chem) |
| ZNFX1 | H00006157-A01 (Novus) |
| CBX5 | H00057169-B01P (Novus) |
| HNRNPA1 | orb15251 (BioOrbyt) |
| SLC1A3 | IQ206 (ImmuQuest) |
| WDR25 | NB110-55631 (Novus) |
| STEAP4 | sc-163525 (Santa Cruz) |
| PTPRM | NB100-80831 (Novus) |
| RAB30 | MBS420071 (MyBioSource) |
| VE | H00027314-M03 (Novus) |
| PCBP1 | Antibodies |
| AREGB | H00005093-M01 (Novus) |
| UBXN6 | sc-5794 (Santa Cruz Biotech) |
| CUGBP1 | H00051035-B01 (Novus) |
| MATR3 | NBP1-19606 (Novus) |
| TCF25 | PA1-27909 (Thermo Scientific, Pierce Antibodies) |
| TOB2 | LS-C81094 (Lifespan Biosciences, Inc.) |
| ECT2 | H00010766-M01 (Novus) |
| GPR113 | NBP1-30872 (Novus) |
| SEL1 | sc-137507 (Santa Cruz Biotech) |
| PAIP2 | MBS420228 (My Biosource LLC) |
| PHF15 | sc-365317 (Santa Cruz Biotech) |
| GSTCD | H00023338-B02 (Novus) |
| INTS12 | H00079807-B01P (Novus) |
| RPS12 | sc-130155 (Santa Cruz Biotech) |
| RPL35A | H00006183-B01 (Novus) |
| KRT86 | H00006165-A01 (Novus) |
| WTAP | LS-C20848 (Lifespan Biosci) |
| SFRS11 | |
| ALS2 | NB100-68245 (Novus) |
| DNAJB9 | sc-160091 (Santa Cruz Biotech) |
| THAP5 | H00057679-M01 (Novus) |
| ARF6 | NBP1-32249 (Novus) |
| BAX | sc-138685 (Santa Cruz Biotech) |
| FTL | H00000382-M01A (Novus) |
| HSPA8 | sc-20068 (Santa Cruz Biotech) |
| KIF12 | MBS619637 (My Biosource, LLC) |
| RAB1A | orb19293 (Biorbyt) |
| SCTR | H00113220-B01 (Novus) |
| TFPI | MBS301542 (My Biosource) |
| ZNF827 | H00006344-B01 (Novus) |
| DUSP16 | sc-81744 (Santa Cruz Biotech) |
| RNLS | LS-C107545 (LifeSpan Biosciences) |
| ARF4 | NB100-848 (Novus) |
| FOSL2 | TA307077 (Origene) |
| GDE1 | H00000379-B01 (Novus) |
| ZNF581 | LS-C66280 (LifeSpan Biosci) |
| TRIB1 | sc-133615 (Santa Cruz Biotech) |
| NFE2L1 | PA1-27759 (Thermo Scientific Pierce Antibodies) |
| RPL22L1 | NB600-1452 (Novus Biologicals) |
| RAB9B | LS-C110256 |
| SYNE2 | sc-100126 (Santa Cruz Biotech) |
| HNF1B | NBP1-03411 (Novus) |
| ALDOA | sc-365431 (Santa Cruz Biotech) |
| CDKN1B | sc-7411 (Santa Cruz Biotech) |
| TBCK | sc-53933 (Santa Cruz Biotech) |
| PARD6B | PA1-38350 (Thermo Scientific) |
| RSPRY1 | H00093627-M01 (Novus Biologicals) |
| BANF1 | sc-133205 (Santa Cruz Biotech) |
| EIF1AD | H00089970-B01 (Novus) |
| ARPC5L | NBP1-02976 (Novus) |
| PAFAH1B1 | sc-242607 (Santa Cruz) |
| BAT2L | A0760-89 (US Biological) |
| TOR1AIP2 | A0760-89 (US Biological) |
| SON | sc-169981 (Santa Cruz Biotech) |
| STK35 | H00002618-M01 (Novus) |
| RBM5 | 46460002 (Novus) |
| HNRNPUL2 | 46460002 (Novus) |
| TTC9C | NBP1-26613 (Novus) |
| RAB5C | LS-C120038 (Lifespan Biosci) |
| EIF4B | sc-138714 (Santa Cruz Biotech) |
| SFXN2 | NBP1-03409 (Novus) |
| ARL3 | TA303495 (Origene Tech) |
| FTSJ1 | H00118980-B01P (Novus) |
| TGIF1 | H00000403-B01 (Novus) |
| RPL35 | H00024140-B01 (Novus) |
| RPL7 | TA300819 (Origene) |
| PDE9A | H00011224-A01 (Novus) |
| PKHD1L1 | NB100-2269 (Novus) |

### Peptides

Peptides that inhibit expression or activity of a gene or a gene product set forth in Table 1 are also provided herein. Peptide libraries can be screened utilizing the screening methods set forth herein to identify peptides that inhibit activity of any of the genes or gene products set forth in Table 1. These peptides can be derived from a protein that binds to any of the genes or gene products set forth in Table 1. These peptides can be any peptide in a purified or non-purified form, such as peptides made of D- and/or L-configuration amino acids (m, for example, the form of random peptide libraries; see Lam et al., Nature 354:82-4, 1991), phosphopeptides (such as in the form of random or partially degenerate, directed phosphopeptide libraries; see, for example, Songyang et al., Cell 72:767-78, 1993).

siRNAs

Short interfering RNAs (siRNAs), also known as small interfering RNAs, are double-stranded RNAs that can induce sequence-specific post-transcriptional gene silencing, thereby decreasing gene expression (See, for example, U.S. Pat. Nos. 6,506,559, 7,056,704, 7,078,196, 6,107,094, 5,898,221, 6,573,099, and European Patent No. 1.144,623, all of which are hereby incorporated in their entireties by this reference). siRNas can be of various lengths as long as they maintain their function. In some examples, siRNA molecules are about 19-23 nucleotides in length, such as at least 21 nucleotides, for example at least 23 nucleotides. In one example, siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends. The direction of dsRNA processing determines whether a sense or an antisense target RNA can be cleaved by the produced siRNA endonuclease complex. Thus, siRNAs can be used to modulate transcription or translation, for example, by decreasing expression of a gene set forth in Table 1, 2, 3 or 4. The effects of siRNAs have been demonstrated in cells from a variety of organisms, including Drosophila, C. elegans, insects, frogs, plants, fungi, mice and humans (for example, WO 02/44321; Gitlin et al., Nature 418:430-4, 2002; Caplen et al., Proc. Natl. Acad. Sci. 98:9742-9747, 2001; and Elbashir et al., Nature 411:494-8, 2001).

Utilizing sequence analysis tools, one of skill in the art can design siRNAs to specifically target one or more of the genes set forth in Table 1 for decreased gene expression. siRNAs that inhibit or silence gene expression can be obtained from numerous commercial entities that synthesize siRNAs, for example, Ambion Inc. (2130 Woodward Austin, Tex. 78744-1832, USA), Qiagen Inc. (27220 Turnberry Lane, Valencia, Calif. USA) and Dharmacon Inc. (650 Crescent Drive, #100 Lafayette, Colo. 80026, USA). The siRNAs synthesized by Ambion Inc., Qiagen Inc. or Dharmacon Inc, can be readily obtained from these and other entities by providing a GenBank Accession No. for the mRNA of any gene set forth herein. In addition, siRNAs can be generated by utilizing Invitrogen's BLOCK-ITT™ RNAi Designer https://rnaidesigner.invitrogen.com/rnaiexpress. siRNA sequences can comprise a 3′TT overhang and/or additional sequences that allow efficient cloning and expression of the siRNA sequences. siRNA sequences can be cloned into vectors and utilized in vitro, ex vivo or in vivo to decrease gene expression. One of skill in the art would know that it is routine to utilize publicly available algorithms for the design of siRNA to target mRNA sequences. These sequences can then be assayed for inhibition of gene expression in vitro, ex vivo or in vivo.

Provided herein is Table 3, which disclosed examples of siRNA molecules for the genes found in Table 1. This is in no way limiting, and one of skill in the art can readily identify further siRNA molecules based on knowledge in the art and the information given herein.

TABLE 3

| HUGO Gene | | |
|---|---|---|
| Name | siRNA | |
| PCBP1 | AAGGCGGGTGTAAGATCAAAGAGAT | (SEQ ID NO: 1) |
| AREGB | GCTCAGGCCATTATGCTGCTGGATT | (SEQ ID NO: 2) |
| UBXN6 | CCTGCATCAAGGAGGCCATTCTCTT | (SEQ ID NO: 3) |
| CUGBP1 | CCACCCAGACCAACCAGATCTTGAT | (SEQ ID NO: 4) |
| PTBP1 | ACTTGTGTCACTAACGGACCGTTTA | (SEQ ID NO: 5) |
| MATR3 | GAGAAAGGTGTAGGGATGATTCTTT | (SEQ ID NO: 6) |
| SNORA74A | GGTTGTCAGCTATCCAGGCTCATGT | (SEQ ID NO: 7) |
| TCF25 | CCTTGCATTTCGATCTCCGTGATGA | (SEQ ID NO: 8) |
| TOB2 | AGCTAGAGATCAAAGTGGCCCTGAA | (SEQ ID NO: 9) |
| ECT2 | AAGGACATTAAAGTGGGCTTTGTAA | (SEQ ID NO: 10) |
| GPR113 | GGGAGAGACAAAGCATGGAATGAAA | (SEQ ID NO: 11) |
| SEL1 | GGCTGGCGCCCAATCTGATAACTTT | (SEQ ID NO: 12) |
| PAIP2 | CATTCTCATGAAGATGACAATCCAT | (SEQ ID NO: 13) |
| PHF15 | CAGCCATGAAGATCCCGGACTCATA | (SEQ ID NO: 14) |
| SEC24C | TAGTCACTACCAACTTCCTGGTGAA | (SEQ ID NO: 15) |
| GSTCD | CAAAGAGGTGAGTAGAGATAGTTCA | (SEQ ID NO: 16) |
| INTS12 | TGATTCCAGTTACCGTCCATCTCAA | (SEQ ID NO: 17) |
| IQCG | TGGAAGACTCAAACCTTCCTCCAAA | (SEQ ID NO: 18) |
| KRT86 | CCTGTTGTCTCCACCAGAGTCAGTA | (SEQ ID NO: 19) |
| WTAP | AGAAGAAATACAGTGAGGAGCTTAA | (SEQ ID NO: 20) |
| SFRS11 | GGGCTTCCTGGAGCAAACTTGAACT | (SEQ ID NO: 21) |
| ALS2 | CCAAGGAAAGAGGCCTGGTCCATAT | (SEQ ID NO: 22) |
| DNAJB9 | TGGCCATGAAGTACCACCCTGACAA | (SEQ ID NO: 23) |
| ARF6 | TCTTCGGGAACAAGGAAATGCGGAT | (SEQ ID NO: 24) |
| BAX | CCAAGAAGCTGAGCGAGTGTCTCAA | (SEQ ID NO: 25) |
| HSPA8 | TGCTGTTGTCCAGTCTGATATGAAA | (SEQ ID NO: 26) |
| KIF12 | CATGCCCTGCTCACCCTTTACATCA | (SEQ ID NO: 27) |
| COL27A1 | GCTCCTTCCTCTTTGGGAAGATGAA | (SEQ ID NO: 28) |
| RAB1A | CAAGTTACTTCTGATTGGCGACTCA | (SEQ ID NO: 29) |
| SCTR | CCCGACTATGTGACGTGCTACAAGT | (SEQ ID NO: 30) |
| KBTBD8 | CCTTCCATGCTTGCAGTATTCTTAA | (SEQ ID NO: 31) |
| TFPI | AGTACATGCACTTTGGGCTTCTGTA | (SEQ ID NO: 32) |
| ZNF827 | CCTCACATGTTAGTAGGCAGGAAGA | (SEQ ID NO: 33) |
| DUSP16 | GGCCCATGAGATGATTGGAACTCAA | (SEQ ID NO: 34) |
| RNLS | GAGGCCTCTAAGCTCGCCTATTGAA | (SEQ ID NO: 35) |
| HPSE2 | CAGTAAATGGCAGCCAGTTGGGAAA | (SEQ ID NO: 36) |
| ARF4 | GGATTGGATGCTGCTGGCAAGACAA | (SEQ ID NO: 37) |
| FOSL2 | CAGGATTATCCCGGGAACTTTGACA | (SEQ ID NO: 38) |
| C16ORF62 | CAAAGACAAAGAAAGTGAACCGGAA | (SEQ ID NO: 39) |
| GDE1 | GGCGTGGAGTTGGACATTGAGTTTA | (SEQ ID NO: 40) |
| ZNF581 | CAAGGCCCAACCACTACCTGCT TAT | (SEQ ID NO: 41) |
| TRIB1 | GGAGAGAACCCAGCTTAGACTAGAA | (SEQ ID NO: 42) |
| NFE2L1 | AGGAATACCTTGGATGGCTATGGTA | (SEQ ID NO: 43) |
| RPL22L1 | CGGGAGAAGGTTAAAGTCAATGGCA | (SEQ ID NO: 44) |
| RAB9B | TGAGTGGGAAATCCCTGCTCTTAAA | (SEQ ID NO: 45) |
| SYNE2 | CAGAGAGAAGCAGGCCACTTCTGAT | (SEQ ID NO: 46) |
| HNF1B | TCAAGGGTTACATGCAGCAACACAA | (SEQ ID NO: 47) |
| ALDOA | GACACTCTACCAGAAGGCGGATGAT | (SEQ ID NO: 48) |
| CDKN1B | GAGCCAGCGCAAGTGGAATTTCGAT | (SEQ ID NO: 49) |
| TBCK | CGATGAACTGTTATCATCACCAGAA | (SEQ ID NO: 50) |
| PARD6B | GGTGAAGAGCAAGTTTGGAGCTGAA | (SEQ ID NO: 51) |
| TMBIM6 | ACGGACTCTGGAACCATGAACATAT | (SEQ ID NO: 52) |
| FAM192A | AGCAACGAAGAGAAGAAGAACTGAA | (SEQ ID NO: 53) |
| RSPRY1 | CCAGGGTCTGTTGTTGACTCTCGAA | (SEQ ID NO: 54) |
| EIF1AD | CCACCAAGAGGAAGCATGTGGTGAA | (SEQ ID NO: 55) |
| PAFAH1B1 | TGGCTATGAAGAGGCATATTCAGTT | (SEQ ID NO: 56) |
| BAT2L | ACTCGACTCTCAGCCTGTTTGATAA | (SEQ ID NO: 57) |
| TOR1AIP2 | TGGACAACAGTGGTTCCCTAGTTTA | (SEQ ID NO: 58) |
| GART | CCGAGTACTTATAATTGGCAGTGGA | (SEQ ID NO: 59) |
| SON | CAGCACCATGGATTCTCAGATGTTA | (SEQ ID NO: 60) |
| STK35 | ACGGCAACAAGAGCTCGCAGCTTTA | (SEQ ID NO: 61) |
| RBM5 | GACCGATCCGAAGATGGCTACCATT | (SEQ ID NO: 62) |
| HNRNPUL2 | GAACATGGCCGAGCTTACTATGAAT | (SEQ ID NO: 63) |
| TTC9C | GGGAAGTACCGAGATGCTGTGAGTA | (SEQ ID NO: 64) |
| RAB5C | CAGATACATTTGCACGGGCCAAGAA | (SEQ ID NO: 65) |
| C4ORF34 | AGAAGGTGGATTTGATCCCTGTGAA | (SEQ ID NO: 66) |
| EIF4B | GAGGCTGACCTGTCTGGCTTTAACA | (SEQ ID NO: 67) |
| SFXN2 | GAGGCTGACCTGTCTGGCTTTAACA | (SEQ ID NO: 68) |
| ARL3 | CAGACCAGGAGGTGAGAATACTTCT | (SEQ ID NO: 69) |
| FTSJ1 | CGTCAAAGGACAAGCGGGATGTCTA | (SEQ ID NO: 70) |
| TGIF1 | CAGATTCTTCGGGATTGGCTGTATG | (SEQ ID NO: 71) |
| SNRPD3 | GAGGGCCACATTGTGACATGTGAGA | (SEQ ID NO: 72) |
| C22ORF13 | CCGGGTGAATCAGCTGTTTGCACAA | (SEQ ID NO: 73) |
| RPL35 | CCAAGCTCTCCAAGATACGAGTCGT | (SEQ ID NO: 74) |
| HNRNPF | GCCGCAGGTGTCCATTTCATCTACA | (SEQ ID NO: 75) |
| PDE9A | TCGATGGACGCATTCAGAAGGTAAT | (SEQ ID NO: 76) |
| NUDCD1 | CAGTGTCTACTATATTGATACCCTT | (SEQ ID NO: 77) |
| PKHD1L1 | CAGTTGGGTAGATTCAGCTTCCTAT | (SEQ ID NO: 78) |
| HIST2H2AB | TCGAGTACCTGACCGCGGAAATTCT | (SEQ ID NO: 79) |
| BOLA1 | GAGAGAACTCTCAGCTGGACACTAG | (SEQ ID NO: 80) |
| AGAP1 | GATGCCTTCGTGAACAGCCAGGAAT | (SEQ ID NO: 81) |
| STAU2 | GATCCAAAGCCATTCCCAAATTATA | (SEQ ID NO: 82) |
| SLCO1A2 | GAAGATGTTTCTGTTGGCAATAACA | (SEQ ID NO: 83) |
| PCID2 | CAGCAGAGATGGAGCATCTTGTGCA | (SEQ ID NO: 84) |
| ITPRIP | CAGATTCCCTGTACCTGGACACGAT | (SEQ ID NO: 85) |
| CTNND1 | CCCAGGATCACAGTCACCTTCTATA | (SEQ ID NO: 86) |
| SF3A2 | CCCTGGAGACCATCGACATCAACAA | (SEQ ID NO: 87) |
| PLEKHJ1 | TGGTGAAGCTGGTGGTGAATTTCCT | (SEQ ID NO: 88) |
| PPP1R9A | GGGAGGAAATATGGCTCCAATGTCA | (SEQ ID NO: 89) |
| HIPK3 | CCAAGCAGTTGTGTATTCCAGGAAA | (SEQ ID NO: 90) |
| ASB6 | CCTGGACCTTGGAGCTGATGTCAAT | (SEQ ID NO: 91) |
| SENP3 | AAGATGGACTCAGGTGGACTCCAAA | (SEQ ID NO: 92) |
| TNFSF12 | TGCGCCTTTCCTGAACCGACTAGTT | (SEQ ID NO: 93) |
| TNFSF13 | CCAGCCTCATCTCCTTTCTTGCTAG | (SEQ ID NO: 94) |
| TNFSF12- | GAGGAAGCCAGAATCAACAGCTCCA | (SEQ ID NO: 95) |
| TNFSF13 | | |
| MIR505 | TGGACATCGGTGGAACGCTGGTTAA | (SEQ ID NO: 96) |
| PANK1 | TGGACATCGGTGGAACGCTGGTTAA | (SEQ ID NO: 97) |
| ACTN4 | GAAGAGATTGTGGACGGCAACGCAA | (SEQ ID NO: 98) |
| [symbol] | [symbol] | [symbol] |
| TBL1XR1 | CATGGCACTGAAGAGGGACAGCAAA | (SEQ ID NO: 100) |
| FLRT3 | GCTGTTCCTTCAAGTAGCACCTCTA | (SEQ ID NO: 101) |
| SLC25A37 | CGGTGAAGACACGAATGCAGAGTTT | (SEQ ID NO: 102) |
| SNORD46 | ACCTGTGTGCCACTTGCCAATGCAA | (SEQ ID NO: 103) |
| SNORD55 | TGTATGATGACAACTCGGTAATGCT | (SEQ ID NO: 104) |
| RBM3 | TCTTCGTGGGAGGGCTCAACTTTAA | (SEQ ID NO: 105) |
| RANGAP1 | CATTGCCAAGCTGGCAGAGACACTT | (SEQ ID NO: 106) |
| BMPR2 | CCCAATGGATCTTTATGCAAGTATT | (SEQ ID NO: 107) |
| DHX9 | CAGAGCAAATAAGCATGGACCTCAA | (SEQ ID NO: 108) |
| SREBF2 | CGAGATGCTGCAATTTGTCAGTAAT | (SEQ ID NO: 109) |
| MARCH7 | CAGATTCATCTTGGAGGCATAGTCA | (SEQ ID NO: 110) |
| CSRP1 | GGGTGTGTCAGAAGACGGTTTACTT | (SEQ ID NO: 111) |
| AP1G1 | CAGGAGAGGTAGAGAAGCTCCTGAA | (SEQ ID NO: 112) |
| AHCY | CAGTGGTCCAGCTGCAACATCTTCT | (SEQ ID NO: 113) |
| CDH6 | CAGGATAGAGGAGATGGATCACTTA | (SEQ ID NO: 114) |
| PABPN1 | AGGAGCTACAGAACGAGGTAGAGAA | (SEQ ID NO: 115) |
| PPP1R3E | GCCAACTCTGTGGGCAGAATGCTAA | (SEQ ID NO: 116) |
| NPAS2 | GGGACCAGTTCAATGTTCTCATCAA | (SEQ ID NO: 117) |
| APBBlIP | CAAGTGAAGACATAGACCAAATGTT | (SEQ ID NO: 118) |
| SETD5 | CCCGAACTCTGAAGGAGAAACTGTA | (SEQ ID NO: 119) |
| PARG | CAAGACAGCGGAATCAGAAAGTTTG | (SEQ ID NO: 120) |
| DR1 | CCTCGTCTGGCAACGATGATGATCT | (SEQ ID NO: 121) |
| LEPREL1 | CAGATGATGAGGATGTCCTAGACAA | (SEQ ID NO: 122) |
| DIAPH3 | TCCCAACCTGAAGACTGCATTTGCA | (SEQ ID NO: 123) |
| CPS1 | CAGATTCTCACAATGGCCAACCCTA | (SEQ ID NO: 124) |
| PKM2 | TAGTGAAGCCGGGACTGCCTTCATT | (SEQ ID NO: 125) |
| SEMA3F | CCCACTTCTTCAACTTCCTGCTCAA | (SEQ ID NO: 126) |
| C20ORF111 | AAAGCTCTACTTCTCTCGATGCTAA | (SEQ ID NO: 127) |
| MANBAL | AGAACCTGCTACGGTACGGACTCTT | (SEQ ID NO: 128) |
| [symbol] | [symbol] | [symbol] |
| PFAS | ACACTCGGAGGAAACTGCAAGGGAA | (SEQ ID NO: 130) |
| C17ORF68 | GAGGATGCTCAGGTCTTCATCCAAA | (SEQ ID NO: 131) |
| CSGALNACT2 | CAACAAAGAGCAAGCACCTAGTGAT | (SEQ ID NO: 132) |
| [symbol] | [symbol] | [symbol] |
| RAB30 | CAAGGAGCCACAATTGGAGTTGATT | (SEQ ID NO: 141) |
| SNORA70 | TCCTTGGTAGTGTACGCAGCCTGTT | (SEQ ID NO: 142) |
| RNF128 | GAGTGCCTATGTGACTGTGACTTAT | (SEQ ID NO: 143) |
| RFWD3 | TCGTTGCATTCAATGACCAACTTCA | (SEQ ID NO: 144) |
| XRCC6 | TGTCAGGGTGGGAGTCATATTACAA | (SEQ ID NO: 145) |

[symbol]

indicates data missing or illegible when filed

shRNA

shRNA (short hairpin RNA) is a DNA molecule that can be cloned into expression vectors to express siRNA (typically 19-29 nt RNA duplex) for RNAi interference studies. shRNA has the following structural features: a short nucleotide sequence ranging from about 19-29 nucleotides derived from the target gene, followed by a short spacer of about 4-15 nucleotides (i.e. loop) and about a 19-29 nucleotide sequence that is the reverse complement of the initial target sequence.

### Antisense Nucleic Acids

Generally, the term “antisense” refers to a nucleic acid molecule capable of hybridizing to a portion of an RNA sequence (such as mRNA) by virtue of some sequence complementarity. The antisense nucleic acids disclosed herein can be oligonucleotides that are double-stranded or single-stranded, RNA or DNA or a modification or derivative thereof, which can be directly administered to a cell (for example by administering the antisense molecule to the subject), or which can be produced intracellularly by transcription of exogenous, introduced sequences (for example by administering to the subject a vector that includes the antisense molecule under control of a promoter).

Antisense nucleic acids are polynucleotides, for example nucleic acid molecules that are at least 6 nucleotides in length, at least 10 nucleotides, at least 15 nucleotides, at least 20 nucleotides, at least 100 nucleotides, at least 200 nucleotides, such as 6 to 100 nucleotides. However, antisense molecules can be much longer. In particular examples, the nucleotide is modified at one or more base moiety, sugar moiety, or phosphate backbone (or combinations thereof), and can include other appending groups such as peptides, or agents facilitating transport across the cell membrane (Letsinger et al., Proc. Natl. Acad. Sci. USA 1989, 86:6553-6; Lemaitre et al., Proc. Natl. Acad. Sci. USA 1987, 84:648-52; WO 88/09810) or blood-brain barrier (WO 89/10134), hybridization triggered cleavage agents (Krol et al., BioTechniques 1988, 6:958-76) or intercalating agents (Zon, Pharm. Res. 5:539-49, 1988). Additional modifications include those set forth in U.S. Pat. Nos. 7,176,296; 7,329,648; 7,262,489, 7,115,579; and 7,105,495.

Examples of modified base moieties include, but are not limited to: 5-fluorouracil, 5-bromouracil, 5-chlorouracil, 5-iodouracil, hypoxanthine, xanthine, acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl)uracil, and 2,6-diaminopurine.

Examples of modified sugar moieties include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

In a particular example, an antisense molecule is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-41, 1987). The oligonucleotide can be conjugated to another molecule, such as a peptide, hybridization triggered cross-linking agent, transport agent, or hybridization-triggered cleavage agent. Oligonucleotides can include a targeting moiety that enhances uptake of the molecule by host cells. The targeting moiety can be a specific binding molecule, such as an antibody or fragment thereof that recognizes a molecule present on the surface of the host cell.

In a specific example, antisense molecules that recognize a nucleic acid set forth herein, include a catalytic RNA or a ribozyme (for example see WO 90/11364; WO 95/06764; and Sarver et al., Science 247:1222-5, 1990). Conjugates of antisense with a metal complex, such as terpyridylCu (II), capable of mediating mRNA hydrolysis, are described in Bashkin et al. (Appl. Biochem Biotechnol. 54:43-56, 1995). In one example, the antisense nucleotide is a 2′-O-methylribonucleotide (Inoue et al., Nucl. Acids Res. 15:6131-48, 1987), or a chimeric RNA-DNA analogue (Inoue et al., FEBS Lett. 215:327-30, 1987). Antisense molecules can be generated by utilizing the Antisense Design algorithm of Integrated DNA Technologies, Inc. (1710 Commercial Park, Coralville, Iowa 52241 USA; http://www.idtdna.com/Scitools/Applications/AntiSense/Antisense.aspx.

Any antisense sequence that is not the full length mRNA for any of the genes listed in Table 1 can be used as antisense sequences. It is known to those of skill in the art that once a mRNA sequence is routinely obtained for any of the genes set forth in Table 1, it is routine to walk along the mRNA sequence to generate antisense sequences that decrease expression of the gene. Therefore, the methods of the present invention can utilize any antisense sequence that decreases the expression of a gene set forth in Table 1.

Provided herein are examples of antisense nucleic acids corresponding to the genes of Table 1. This is in no way to be construed as limiting, as one of skill in the art can readily identify antisense nucleic acids.

TABLE 4

| HUGO | | |
|---|---|---|
| Gene | | |
| Name | Antisense | |
| PCBP1 | 5′ TCTTACACCCGCCTTTCCCA 3′ | (SEQ ID NO: 146) |
| AREGB | 5′ GGCTCTCATTGGTCCTTCGCA 3′ | (SEQ ID NO: 147) |
| UBXN6 | 5′ CTGTTCCTTGTGCCTCTCCA 3′ | (SEQ ID NO: 148) |
| CUGBP1 | 5′ ACATCTCTCCACCCTTCCCT 3′ | (SEQ ID NO: 149) |
| PTBP1 | 5′ GGGTGTGACTCTCTCTGGGT 3′ | (SEQ ID NO: 150) |
| MATR3 | 5′ CCTGCCACTATTTCCTCCCT 3′ | (SEQ ID NO: 151) |
| SNORA74A | 5′ ACACCATCACAGGCACCACA 3′ | (SEQ ID NO: 152) |
| TCF25 | 5′ CCTTCCTTTGTTCCCTGGTGC 3′ | (SEQ ID NO: 153) |
| TOB2 | 5′ GTCGCTCTCCTTTCCTTCCC 3′ | (SEQ ID NO: 154) |
| ECT2 | 5′ CCTGTTCATTCCGCCTTTCCC 3′ | (SEQ ID NO: 155) |
| GPR113 | 5′ CTCTGTCCCAACTCCTCCTCT 3′ | (SEQ ID NO: 156) |
| SEL1 | 5′ GCTTTCCGTCCACACCATCT 3′ | (SEQ ID NO: 157) |
| PPIA | 5′ TCCCACGTCAGCCTCCAGAT 3′ | (SEQ ID NO: 158) |
| PAIP2 | 5′ TCTTCTTCCCATAACTCCTCT 3′ | (SEQ ID NO: 159) |
| PHF15 | 5′ GGTGGTGGTGGTGGTGGTTT 3′ | (SEQ ID NO: 160) |
| SEC24C | 5′ GTCTCTGTTTCCCTTGTGTCT 3′ | (SEQ ID NO: 161) |
| GSTCD | 5′ TCCCTGTTGCTTTCCTTCCT 3′ | (SEQ ID NO: 162) |
| INTS12 | 5′ GTTCCACTATTTCCATTCCCA 3′ | (SEQ ID NO: 163) |
| RPS12 | 5′ GTCCATTACACCTCCAGCAG 3′ | (SEQ ID NO: 164) |
| SNORD101 | 5′ GGGTATCCGACAATTAAAGTC 3′ | (SEQ ID NO: 165) |
| RPL35A | 5′ GACCACAGCCTTCCAGACAT 3′ | (SEQ ID NO: 166) |
| IQCG | 5′ AGTCCTTCCCACCTCCAGA 3′ | (SEQ ID NO: 167) |
| KRT86 | 5′ TTCTCCACCTCAGCCGTCAG 3′ | (SEQ ID NO: 168) |
| WTAP | 5′ TCCCACTCACTGCTTTCTCCT 3′ | (SEQ ID NO: 169) |
| HIST2H2AA | 5′ GTCTTCTTGTTGTCCCGAGCC 3′ | (SEQ ID NO: 170) |
| 3 | | |
| SNORD58B | 5′ GTGTCCTAAGAAATGCCATCA 3′ | (SEQ ID NO: 171) |
| SNORD58C | 5′ AGTCATCACAGCAACCACA 3′ | (SEQ ID NO: 172) |
| SFRS11 | 5′ GCCACTCCTGCTTCTTCGTC 3′ | (SEQ ID NO: 173) |
| ALS2 | 5′ GCACAGTCTCTCCCTTAGTT 3′ | (SEQ ID NO: 174) |
| DNAJB9 | 5′ GCCACCACCTCAGAAGACCT 3′ | (SEQ ID NO: 175) |
| THAP5 | 5′ TCTCTCCAGCCCAACTCTCT 3′ | (SEQ ID NO: 176) |
| ARF6 | 5′ ACTCCTCCTCCCAGTTCTGC 3′ | (SEQ ID NO: 177) |
| BAX | 5′ AGTCTCACCCAACCACCCTG 3′ | (SEQ ID NO: 178) |
| FTL | 5′ GCCTTCCAGAGCCACATCATC 3′ | (SEQ ID NO: 179) |
| SNORA73B | 5′ CCTATGCCACTTGGACAGAGC 3′ | (SEQ ID NO: 180) |
| RNU105A | 5′ CCTATGCCACTTGGACAGAGC 3′ | (SEQ ID NO: 181) |
| HSPA8 | 5′ CCTTGTCCCTCTGCTTCTC 3′ | (SEQ ID NO: 182) |
| KIF12 | 5′ GACTCCCAAATTCCACCACCC 3′ | (SEQ ID NO: 183) |
| COL27A1 | 5′ CCTTGTGTCCCTTTCGTCCC 3′ | (SEQ ID NO: 184) |
| RAB1A | 5′ AGCAGCCATATCCCAAGCCC 3′ | (SEQ ID NO: 185) |
| SCTR | 5′ GCATTCCACCTCCACCATCC 3′ | (SEQ ID NO: 186) |
| KBTBD8 | 5′ TCCCGTGATCCACTTCCACT 3′ | (SEQ ID NO: 187) |
| TFPI | 5′ GTGTGTTCTTCATCTTCCTC 3′ | (SEQ ID NO: 188) |
| ZNF827 | 5′ TCTGTCCCTCCTCCTTCCCT 3′ | (SEQ ID NO: 189) |
| DUSP16 | 5′ CTCTTTCTCTTCCACCCTCCC 3′ | (SEQ ID NO: 190) |
| RNLS | 5′ AGTCCTCAGCCTTGTCCCAC 3′ | (SEQ ID NO: 191) |
| HPSE2 | 5′ CTTCCCTCTCCCAAATCCACC 3′ | (SEQ ID NO: 192) |
| ARF4 | 5′ TCTTGACCACCAACATCCCA 3′ | (SEQ ID NO: 193) |
| FOSL2 | 5′ CCTCTCCCTCTCTCTCTCTCT 3′ | (SEQ ID NO: 194) |
| C160RF62 | 5′ CTCCACCCGCACACTCTCTT 3′ | (SEQ ID NO: 195) |
| GDE1 | 5′ TCCTCCCACCTCAACCTCCT 3′ | (SEQ ID NO: 196) |
| ZNF581 | 5′ CTCCCTCTGTGACTCCTCGT 3′ | (SEQ ID NO: 197) |
| TRIB1 | 5′ CCTCTGTCCTCCCTCTTCTC 3′ | (SEQ ID NO: 198) |
| NFE2L1 | 5′ GCCACCTTCCCTTCCTCTCA 3′ | (SEQ ID NO: 199) |
| RPL22L1 | 5′ TCTTGTCCTCCCTAATCTCCT 3′ | (SEQ ID NO: 200) |
| RAB9B | 5′ TCCCAACTCCACCATCACCC 3′ | (SEQ ID NO: 201) |
| HNF1B | 5′ GTTCCTTGTCTCCCACCTCC 3′ | (SEQ ID NO: 202) |
| ALDOA | 5′ GTAGTCTCGCCATTTGTCCCT 3′ | (SEQ ID NO: 203) |
| CDKN1B | 5′ CCCTTCTCCACCTCTTGCCA 3′ | (SEQ ID NO: 204) |
| TBCK | 5′ TTTCACCCTCTACCCTCCC 3′ | (SEQ ID NO: 205) |
| PARD6B | 5′ GCTGTCTTCATCCTCTGGCTC 3′ | (SEQ ID NO: 206) |
| TMBIM6 | 5′ ACTCCTCTCACTTCCCGCCT 3′ | (SEQ ID NO: 207) |
| FAM192A | 5′ ATCTCCCTCTCCCTCCTCCA 3′ | (SEQ ID NO: 208) |
| RSPRY1 | 5′ TCCCGTGGTTGGCTCCTTGT 3′ | (SEQ ID NO: 209) |
| BANF1 | 5′ GACTTCACCAATCCCAGCCAG 3′ | (SEQ ID NO: 210) |
| EIF1AD | 5′ CCTCCTCTTCACTCTCCTCCT 3′ | (SEQ ID NO: 211) |
| ARPC5L | 5′ TCTCCCGTTTACCACAGCCCT 3′ | (SEQ ID NO: 212) |
| PAFAH1B1 | 5′ GTCCTTCACTCCCATCCACC 3′ | (SEQ ID NO: 213) |
| BAT2L | 5′ GTCCACTCCCTCCATCACCA 3′ | (SEQ ID NO: 214) |
| TOR1AIP2 | 5′ TGATCCTCCCACCTCAGCCT 3′ | (SEQ ID NO: 215) |
| IFRG15 | 5′ TGATCCTCCCACCTCAGCCT 3′ | (SEQ ID NO: 216) |
| MIR7-1 | 5′ GTCTTCCACACAGAACTAGGC 3′ | (SEQ ID NO: 217) |
| GART | 5′ GTTCCCTTCCTCCACTGCCA 3′ | (SEQ ID NO: 218) |
| SON | 5′ TCTGGCTCTGGTGGTGGTTCT 3′ | (SEQ ID NO: 219) |
| STK35 | 5′ TGCTGCTGTTCCCACCCTCT 3′ | (SEQ ID NO: 220) |
| RBM5 | 5′ GTCTTGCTCTCCCTCTCGTC 3′ | (SEQ ID NO: 221) |
| HNRNPUL2 | 5′ GTCCTCCTCCTCCTCCTCTT 3′ | (SEQ ID NO: 222) |
| TTC9C | 5′ GCTGGTTCCCTTCCTCCTTGT 3′ | (SEQ ID NO: 223) |
| RAB5C | 5′ CCTCCACTTCCGCCTTTCAG 3′ | (SEQ ID NO: 224) |
| C4ORF34 | 5′ CCTCCACTTCCGCCTTTCAG 3′ | (SEQ ID NO: 225) |
| EIF4B | 5′ TCTCTGTTCCCTCCGTCTCCT 3′ | (SEQ ID NO: 226) |
| SFXN2 | 5′ CCATCCTGCTCTTCTCCACC 3′ | (SEQ ID NO: 227) |
| ARL3 | 5′ ACCTCCCACCTCTCCTTACA 3′ | (SEQ ID NO: 228) |
| FTSJ1 | 5′ GGCTGTGTGGGTGGAGTGTA 3′ | (SEQ ID NO: 229) |
| TGIF1 | 5′ TCTCCCTCGCCCAACTCTCT 3′ | (SEQ ID NO: 230) |
| SNRPD3 | 5′ GTCCCATTCCACGTCCTCTTC 3′ | (SEQ ID NO: 231) |
| C22ORF13 | 5′ TGCCTCTGTGACCCTCCCTA 3′ | (SEQ ID NO: 232) |
| RPL35 | 5′ GGCTCTTGTCTTCTTGGGTC 3′ | (SEQ ID NO: 233) |
| HNRNPF | 5′ TCCTCTCCTTGTGTTTCCCT 3′ | (SEQ ID NO: 234) |
| RPL7 | 5′ CCTCCTTCTTCTTCTCTTCT 3′ | (SEQ ID NO: 235) |
| PDE9A | 5′ GCCAGCTCCTCCCTCATCTT 3′ | (SEQ ID NO: 236) |
| NUDCD1 | 5′ CCTGCCTTCCTTTCTGTTGT 3′ | (SEQ ID NO: 237) |
| PKHD1L1 | 5′ TCTCTTCTCCCTCCTGCCA 3′ | (SEQ ID NO: 238) |
| HIST2H2AB | 5′ GTCTTCTTGTTGTCCCGAGCC 3′ | (SEQ ID NO: 239) |
| BOLA1 | 5′ TTCTTGTTCCCACCCAGGCA 3′ | (SEQ ID NO: 240) |
| AGAP1 | 5′ CCACCGTTCCTTCTCTTCCCT 3′ | (SEQ ID NO: 241) |
| STAU2 | 5′ GTCCCAAGTCCAGAGGCAGT 3′ | (SEQ ID NO: 242) |
| HSP90AB1 | 5′ TCTCCACCTCCTCCTCTCCA 3′ | (SEQ ID NO: 243) |
| SLCO1A2 | 5′ TCTCCCACCCTTCCTTACTCC 3′ | (SEQ ID NO: 244) |
| PCID2 | 5′ ATGTCCACGTCCTCCACCT 3′ | (SEQ ID NO: 245) |
| RPL26 | 5′ GTTTCTTCCTTGTATTTGCCC 3′ | (SEQ ID NO: 246) |
| ITPRIP | 5′ CCTTCTATGTCCCTTCCCTTC 3′ | (SEQ ID NO: 247) |
| CTNND1 | CTCTCTCTCTCCCTCTCTCT | (SEQ ID NO: 248) |
| SF3A2 | 5′ TCCTCCTCGTTTCGCCACCT 3′ | (SEQ ID NO: 249) |
| PLEKHJ1 | 5′ GCCTCCATCCACTCCTGACA 3′ | (SEQ ID NO: 250) |
| PPP1R9A | 5′ CCTCCTCACCACACATTCCCT 3′ | (SEQ ID NO: 251) |
| HIPK3 | 5′ CCTCCTCACCACACATTCCCT 3′ | (SEQ ID NO: 252) |
| ASB6 | 5′ ACACCTGCCACCCACTTCCT 3′ | (SEQ ID NO: 253) |
| SENP3 | 5′ TCCTCCTCCTCCTCCTCTTCT 3′ | (SEQ ID NO: 254) |
| TNFSF12 | ′ CTTCCTCCCAGCCACTCACT 3′ | (SEQ ID NO: 255) |
| TNFSF13 | 5′ GTTCCTGCACATTCCCTCTCC 3′ | (SEQ ID NO: 256) |
| TNFSF12- | 5′ CTTCCTCCCAGCCACTCACT 3′ | (SEQ ID NO: 257) |
| TNFSF13 | | |
| MIR505 | 5′ CATCAATACTTCCTGGCTCCC 3′ | (SEQ ID NO: 258) |
| PANK1 | 5′ CCCTCCCTTCCTCTCCACTT 3′ | (SEQ ID NO: 259) |
| MIR107 | 5′ CTTGAACTCCATGCCACAA 3′ | (SEQ ID NO: 260) |
| ACTN4 | 5′ GCTTCCTTCCCGTCAGTCCA 3′ | (SEQ ID NO: 261) |
| NUMA1 | 5′ CCACCTTCTCCTTTGCCTCCT 3′ | (SEQ ID NO: 262) |
| TBL1XR1 | 5′ CCTACCCTCCTTCCATCCCT 3′ | (SEQ ID NO: 263) |
| PA2G4 | 5′ TGTCTCCCTTCCCAGCCACA 3′ | (SEQ ID NO: 264) |
| FLRT3 | 5′ GTCATCCTTTCTTCTCCTCCC 3′ | (SEQ ID NO: 265) |
| SLC25A37 | 5′ GTCCTTCCTGTCCACCACCA 3′ | (SEQ ID NO: 266) |
| RPS8 | 5′ CTTGTGCCAGTTGTCCCGAG 3′ | (SEQ ID NO: 267) |
| SNORD38A | 5′ GCTCTCATCTCTCTCCCTTCA 3′ | (SEQ ID NO: 268) |
| SNORD38B | 5′ CTCCTCAGACACACTTTATC 3′ | (SEQ ID NO: 269) |
| SNORD46 | 5′ GCCACAACCACGCCTAAGGA 3′ | (SEQ ID NO: 270) |
| SNORD55 | 5′ GCTCAGCTCTCCAAGGTTG 3′ | (SEQ ID NO: 271) |
| RBM3 | 5′ TACCTGCCACTCCCATAGCC 3′ | (SEQ ID NO: 272) |
| RANGAP1 | ′ TCCTCCTCCTCCTCCTCTTCT 3′ | (SEQ ID NO: 273) |
| BMPR2 | 5′ TTCTCCTGTCTCTGCCTCCC 3′ | (SEQ ID NO: 274) |
| DHX9 | 5′ CACCTCCTCTTCCCTGTCCA 3′ | (SEQ ID NO: 275) |
| SREBF2 | 5′ CCCTGCCACCTATCCTCTCA | (SEQ ID NO: 276) |
| | 3′ | |
| MARCH7 | 5′ TCTCTTCCCTCTCGCCTCCT 3′ | (SEQ ID NO: 277) |
| RPL29 | 5′ GGTTGTGTGTGGTGTGGTTCT 3 | (SEQ ID NO: 278) |
| AP1G1 | 5′ TTCGCTCCCTGTCCTCCTCT 3′ | (SEQ ID NO: 279) |
| AHCY | 5′ TACTCTGTTCCCGCTGCCAC 3′ | (SEQ ID NO: 280) |
| CDH6 | 5′ TCCTCCTCTCCACCACCTTC 3′ | (SEQ ID NO: 281) |
| PABPN1 | 5′ TCCCACTTCCTCCCATTCCCT 3′ | (SEQ ID NO: 282) |
| PPP1R3E | 5′ TCCTCCACCTCCCGTTGCTT 3′ | (SEQ ID NO: 283) |
| NPAS2 | 5′ GTCCCTGGCTGTTGTGAGT 3′ | (SEQ ID NO: 284) |
| APBBlIP | 5′ CACTCATCATGCCCGTCCCT 3′ | (SEQ ID NO: 285) |
| SETD5 | 5′ TCCACTCCCAATGCTCTCCC 3′ | (SEQ ID NO: 286) |
| PARG | 5′ GTCGTCCCTTTCACTCCCATC 3′ | (SEQ ID NO: 287) |
| DR1 | 5′ GCAGTTCACCACCAGCTCTC 3′ | (SEQ ID NO: 288) |
| LEPREL1 | 5′ CCACAGCACACCTCTTTCCCT 3′ | (SEQ ID NO: 289) |
| DIAPH3 | GTCTCTCTTTCTTGCTCCCTG | (SEQ ID NO: 290) |
| CPS1 | 5′ CCACCACCGTCTTCTTGCCA 3′ | (SEQ ID NO: 291) |
| PKM2 | 5′ GTTCTTTCCCTTCTCTCCCA 3′ | (SEQ ID NO: 292) |
| SEMA3F | 5′ CCCTTCTTCCCTTCTCTGTGT 3′ | (SEQ ID NO: 293) |
| C20ORF111 | 5′ ACTCTCCTCCTCTCCATCCT 3′ | (SEQ ID NO: 294) |
| MANBAL | 5′ CCTCCTCTTCTACCGCTTCT 3′ | (SEQ ID NO: 295) |
| LIMCH1 | 5′ GCCATTTGTCGTCCTCTTCCC 3′ | (SEQ ID NO: 296) |
| PFAS | 5′ CGTTCCTCCTCTGCCACACA 3′ | (SEQ ID NO: 297) |
| C17ORF68 | 5′ TCCCTGCTTACTCCTTACCCT 3′ | (SEQ ID NO: 298) |
| CSGALNAC | 5′ GCGTCTCTATTTCCTCCCTG 3′ | (SEQ ID NO: 299) |
| T2 | | |
| FLVCR2 | 5′ ATTCTCTGCCACCCTGTCCC 3′ | (SEQ ID NO: 300) |
| HSPE1 | 5′ TCCTGCCATTACTCCCTCCG 3′ | (SEQ ID NO: 301) |
| RPL27 | ′5 GTCAATTCCAGCCACCAGAG 3′ | (SEQ ID NO: 302) |
| SNORD12 | 5′ ACAGGGTCGATCTGATGGG 3′ | (SEQ ID NO: 303) |
| SNORD12B | 5′ GCTCAAGCTGGCATATCAGAC 3′ | (SEQ ID NO: 304) |
| SNORD12C | 5′ AGCATTGTCGATCTGATGGG 3′ | (SEQ ID NO: 305) |
| ZNFX1 | 5′ CCTCACCACCTCTTCCTCCT 3′ | (SEQ ID NO: 306) |
| CBX5 | 5′ TCCCTGCCTCACGATTCCCT 3′ | (SEQ ID NO: 307) |
| HNRNPA1 | 5′ TCCCTGTCACTTCTCTGGCTC 3 | (SEQ ID NO: 308) |
| SLC1A3 | 5′ TTCCTTTGTGCCCTTCCCAG 3′ | (SEQ ID NO: 309) |
| WDR25 | 5′ CCTCTGTGCTCGTTCCTCTC 3′ | (SEQ ID NO: 310) |
| STEAP4 | 5′ GAGTTCCTTTCCCAGCCCT 3′ | (SEQ ID NO: 311) |
| NPTX1 | 5′ GCTCTCTCTTGACCTCCTCC 3′ | (SEQ ID NO: 312) |
| PTPRM | 5′ ACTCCTCTTGCCACTTGTCCA 3′ | (SEQ ID NO: 313) |
| RAB30 | 5′ TCTATCTCCCGCAGCCACTCA 3 | (SEQ ID NO: 314) |
| SNORA70 | 5′ GTCCCTTAGAGCAACCCATAC 3′ | (SEQ ID NO: 315) |
| RNF128 | 5′ CCTACCACTCCCATAGCATT 3 | (SEQ ID NO: 316) |
| RPS10 | 5′ GCCACCATGACTCCCTCCTT 3′ | (SEQ ID NO: 317) |
| GNAL | 5′ TTTCCCAGACTCACCAGCCC 3′ | (SEQ ID NO: 318) |
| CHMP1B | 5′ CTTTCTCCTTCCTTCCAGTCC 3′ | (SEQ ID NO: 319) |
| RFWD3 | 5′ TCTCCACAGTGTCTTCTCCCA 3′ | (SEQ ID NO: 320) |
| XRCC6 | 5′ TCTTCAGCCCACTCTTCAGCC 3′ | (SEQ ID NO: 321) |

### Morpholinos

Morpholinos are synthetic antisense oligos that can block access of other molecules to small (about 25 base) regions of ribonucleic acid (RNA). Morpholinos are often used to determine gene function using reverse genetics methods by blocking access to mRNA. Morpholinos, usually about 25 bases in length, bind to complementary sequences of RNA by standard nucleic acid base-pairing. Morpholinos do not degrade their target RNA molecules. Instead, Morpholinos act by “steric hindrance”, binding to a target sequence within an RNA and simply interfering with molecules which might otherwise interact with the RNA. Morpholinos have been used in mammals, ranging from mice to humans.

Bound to the 5′-untranslated region of messenger RNA (mRNA), Morpholinos can interfere with progression of the ribosomal initiation complex from the 5′ cap to the start codon. This prevents translation of the coding region of the targeted transcript (called “knocking down” gene expression). Morpholinos can also interfere with pre-mRNA processing steps, usually by preventing the splice-directing snRNP complexes from binding to their targets at the borders of introns on a strand of pre-RNA. Preventing U1 (at the donor site) or U2/U5 (at the polypyrimidine moiety & acceptor site) from binding can cause modified splicing, commonly leading to exclusions of exons from the mature mRNA. Targeting some splice targets results in intron inclusions, while activation of cryptic splice sites can lead to partial inclusions or exclusions. Targets of U11/U12 snRNPs can also be blocked. Splice modification can be conveniently assayed by reverse-transcriptase polymerase chain reaction (RT-PCR) and is seen as a band shift after gel electrophoresis of RT-PCR products. Methods of designing, making and utilizing morpholinos are disclosed in U.S. Pat. No. 6,867,349 which is incorporated herein by reference in its entirety.

### Small Molecules

The present invention also provides the design and synthesis of small molecules that inhibit activity of any of the genes or gene products set forth in Table 1. One of skill in the art can search available databases to obtain three dimensional structures of the proteins set forth herein, or three dimensional structures of the relevant domains for the proteins provided herein. For example, the skilled artisan can query the RCSB Protein Databank http://www.rcsb.org/pdb/home/home.do or http://www.rcsb.org for available three dimensional structures. As structures are elucidated, one of skill in the art can search this or other databases to obtain additional structural information for the genes set forth herein. In other instances, crystal structures can be generated for the same purpose. High throughput screening of compound libraries for the identification of small molecules is also contemplated by the present invention. Compound libraries are commercially available. For example, libraries can be obtained from ChemBridge Corporation (San Diego, Calif.), such as a GPCR library, a kinase targeted library (KINACore), or an ion channel library (Ion Channel Set), to name a few. Compound libraries can also be obtained from the National Institutes of Health. For example, the NIH Clinical Collection of compounds that have been used in clinical trials can also be screened. Biofocus DPI (Essex, United Kingdom) also maintains and designs compound libraries that can be purchased for screening. One of skill in the art can select a library based on the protein of interest. For example, a GPCR library can be screened to identify a compound that binds to a G protein coupled receptor. Similarly, a kinase library can be screened to identify a compound that binds to a kinase. Other libraries that target enzyme families can also be screened, depending on the type of enzyme.

Modeling techniques that allow virtual screening of compound libraries are also contemplated herein. For example, Hyperchem software (HyperCube, Inc., Gainesville, Fla.) or AutoDock software (LaJolla, Calif.) can be utilized.

Other methods of decreasing expression and/or activity include methods of interrupting or altering transcription of mRNA molecules by site-directed mutagenesis (including mutations caused by a transposon or an insertional vector). Chemical mutagenesis can also be performed in which a cell is contacted with a chemical (for example ENU) that mutagenizes nucleic acids by introducing mutations into a gene set forth in Table 1. Transcription of mRNA molecules can also be decreased by modulating a transcription factor that regulates expression of any of the genes set forth in Table 1. Radiation can also be utilized to effect mutagenesis.

Provided herein are examples of modulators of the genes/proteins found in Table 1. These are in no way limiting, and are meant to be exemplary only.

TABLE 5

| NAME | Small Molecule Modulators |
|---|---|
| PCBP1 | p-cbp; poly c; tyrosine |
| TOB2 | Mercaptopurine; methotrexate |
| PPIA | cyclosporin a; glyceraldehyde 3-phosphate; heparan sulfate; |
| | tacrolimus |
| PAIP2 | Pabc; adenylate |
| KRT86 | Lysine; glutamate |
| ALS2 | Guanine, superoxide |
| ARF6 | grp-1; gdp; -3,4,5-trisphosphate; pip2; phosphatidylinositol; |
| | butanol; ptdins(3)p |
| BAX | 2,3-DCPE hydrochloride; Gossypol; HA14-1; adpribose; oridonin; noxa3-(4,5- |
| | dimethylthiazol-2-yl)-2,5-diphenyltetrazolium bromide; |
| | gambogic acid; sp 600125; etoposide; cisplatin; mercaptopurine; methotrexate |
| FTL | Ferrozine; ferric ammonium citrate; deferoxamine; haem; fenton; h2o2; |
| | chloroquine |
| HSPA8 | Geldanamycin; 17-(allylamino)-17-demethoxygeldanamycin; |
| | geranylgeranylaeetone; radicicol; sodium arsenite; quercetin; delta-12-pgj2; |
| | cdcl2 |
| SCTR | Demethylasterriquinone Bl; Secretin (human) |
| TFPI | n-methyl-n′-nitrosourea; heparin; 1,3-butadiene |
| RNLS | amine |
| ARF4 | Mercaptopurin; methotrexate |
| FOSL2 | Leucine; pd 98,059; n acetylcysteine |
| NFE2L1 | gamma-glutamylcysteine |
| HNF1B | Nnmt; uric acid; nicotinamide; arginine; tetracycline; retinoic acid |
| ALDOA | fructose-1,6-bisphosphate; fructose 1-phosphate; glyceraldehyde 3-phosphate |
| CDKN1B | ly294002; leptomycin b; eb 1089; 1,25 dihydroxy vitamin d3; mg 132; |
| | ciglitazone; trastuzumab; bromodeoxyuridine; mimosine; Rapamycin; |
| | Docetaxel; Gemcitabine; Pravastatin; tamoxifen; trastuzumab |
| EIF1AD | bafilomycin a1 |
| PAFAH1B1 | Medroxyprogesterone; phospholipid; txb2; leukotriene; endotoxin; |
| | polyacrylamide; arachidonic acid |
| RAB5C | Mercaptopurine; methotrexate |
| EIF4B | Rapamycin; atp; leucine |
| TGIF1 | tgf beta1; cmdb7; vegf; hydroxytamoxifen; retinoic acid; estrogen; |
| | procollagen; threonine; progesterone |
| PDE9A | Cgmp; ibmx |
| SLCO1A2 | estrone sulfate; taurocholate; deltorphin ii; bromosulfophthalein; |
| | fexofenadine; pravastatin; digoxin; probenecid; prostaglandin |
| TNFSF13 | monopril |
| PANK1 | pantothenate |
| NUMA1 | Nocodazole; retinoic acid; paclitaxel |
| PA2G4 | testosterone |
| BMPR2 | Monocrotaline |
| DHX9 | Actinomycin d |
| SREBF2 | Sterol; 3-hydroxy-3-methylglutaryl-coa; cholesterol; squalene; |
| | hydroxycholesterol; n-acetylleucylleucylnorleucinal; acetyl-coa; |
| | lovastatin |
| RPL29 | keratan sulfate; dermatan sulfate; chondroitin sulfate; hyaluronic acid; |
| | butyrate |
| AP1G1 | mannose 6-phosphate; propanil; wortmannin; phenylalanine; alanine |
| AHCY | s-adenosylhomocysteine; neplanocin a; 3-deazaadenosine; aristeromycin; s- |
| | adenosylmethionine; homocysteine; deoxyadenosine; tubercidin; |
| | antimetabolite; mercaptopurine; methotrexate |
| PARG | Mercaptopurine; methotrexate |
| CPS1 | carbamoyl phosphate; n-acetylglutamate |
| PKM2 | Pyruvate; fructose-1,6-bisphosphate; phosphoenolpyruvate; cellulose |
| | acetate; glucose; phenylalanine |
| MANBAL | inosine monophosphate; purine; guanylate |
| HSPE1 | amp-pnp; 8-azido-atp; mgatp; malate; guanidine hydrochloride; pyruvate; |
| | glyceraldehyde 3-phosphate; acyl-coa |
| RPL27 | creatinine |
| SLC1A3 | L-trans-2,4-PDC; DL-TBOA; TFB-TBOA; Glutamate; dihydrokainate; |
| | dl-threo-beta-benzyloxyaspartate; l-trans-pyrrolidine-2,4-dicarboxylic acid; |
| | threo-beta-hydroxyaspartate |
| NPTX1 | milbolerone |

### Pharmaceutical Compositions and Modes of Administration

The present invention provides a method of decreasing infection by a pathogen in a subject by decreasing the expression or activity of a gene or gene product set forth in Table 1, said method comprising administering to the subject an effective amount of a composition that decreases the expression or activity of a gene or a gene product set forth in Table 1.

Also provided is a method of decreasing infection in a subject comprising administering to the subject an effective amount of a composition that decreases expression or activity of a gene or a gene product set forth in Table 1, wherein the composition inhibits infection by two or more respiratory viruses. Also provided is a method of decreasing infection in a subject comprising administering to the subject an effective amount of a composition that decreases expression or activity of a gene or a gene product set forth in Table 1, wherein the composition inhibits infection by three or more respiratory viruses. Also provided is a method of decreasing infection in a subject comprising administering to the subject an effective amount of a composition that decreases expression or activity of a gene or a gene product set forth in Table 1, wherein the composition inhibits infection by four or more respiratory viruses. Also provided is a method of decreasing infection in a subject comprising administering to the subject an effective amount of a composition that decreases expression or activity of a gene or a gene product set forth in Table 1, wherein the composition inhibits infection by five or more respiratory viruses. These can be selected from the group consisting of: a picornavirus, an orthomyxovirus, a paramyxovirus, a coronavirus and an adenovirus. Since picornaviruses, orthomyxoviruses, paramyxoviruses, coronaviruses and adenoviruses are families of viruses, two or more, three or more, four or more, or five or more respiratory viruses can be from the same or from different families. For example, and not to be limiting, the composition can inhibit infection by two or more orthomyxoviruses; two or more picornaviruses; an orthomyxovirus, an adenovirus, and a picornavirus; an orthomyxovirus, a paramyxovirus and an adenovirus; an orthomyxovirus, two picornaviruses and a paramyxovirus; three orthomyxoviruses, a picornavirus and an adenovirus, etc. More particularly, the composition can inhibit infection by two or more, three or more or four or more respiratory viruses selected from the group consisting of an influenza virus, a parainfluenza virus, an adenovirus, a rhinovirus and an RSV virus.

The present invention also provides a method of decreasing infection in a subject comprising administering to the subject an effective amount of a composition that decreases expression or activity of a gene or a gene product set forth in Table 1, wherein the composition inhibits infection by two or more gastrointestinal viruses. The present invention also provides a method of decreasing infection in a subject comprising administering to the subject an effective amount of a composition that decreases expression or activity of a gene or a gene product set forth in Table 1, wherein the composition inhibits infection by three or more gastrointestinal viruses. The present invention also provides a method of decreasing infection in a subject comprising administering to the subject an effective amount of a composition that decreases expression or activity of a gene or a gene product set forth in Table 1, wherein the composition inhibits infection by four or more gastrointestinal viruses. The present invention also provides a method of decreasing infection in a subject comprising administering to the subject an effective amount of a composition that decreases expression or activity of a gene or a gene product set forth in Table 1, wherein the composition inhibits infection by five or more gastrointestinal viruses. These viruses can be selected from the group consisting of: a filovirus, a picornavirus, a calcivirus, a flavivirus or a reovirus. Since filoviruses, picornaviruses, calciviruses, flaviviruses and reoviruses are families of viruses, the composition can inhibit infection by two or more, three or more, four or more, or five or more gastrointestinal viruses from the same or from different families. More particularly, the composition can inhibit infection by two or more, three or more, four or more, or five or more gastrointestinal viruses selected from the group consisting of a reovirus, a Norwalk virus, an Ebola virus, a Marburg virus, a Dengue fever virus, a West Nile virus, a yellow fever virus, a rotavirus and an enterovirus.

The present invention also provides a method of decreasing infection in a subject comprising administering to the subject an effective amount of a composition that decreases expression or activity of a gene or a gene product set forth in Table 1, wherein the composition inhibits infection by one or more pathogens selected from the group consisting of: a picornavirus, an orthomyxovirus, a paramyxovirus, a coronavirus, an adenovirus, and inhibits infection by one or more pathogens selected from the group consisting of: a flavivirus, a filovirus, a calcivirus or a reovirus.

The present invention also provides a method of decreasing infection in a subject comprising administering to the subject an effective amount of a composition that decreases expression or activity of a gene or a gene product set forth in Table 1, wherein the composition inhibits infection by two or more pathogens selected from the group consisting of HIV virus, a pox virus, a herpes virus, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

The present invention also provides a method of decreasing infection in a subject comprising administering to the subject an effective amount of a composition that decreases expression or activity of a gene or a gene product set forth in Table 1 wherein the composition inhibits infection by two or more pathogens selected from the group consisting of: influenza, a pox virus, LCM, Junin virus, Machupo virus, Guanarito virus, Lassa Fever virus, hantavirus, Rift Valley Fever virus Ebola virus, Marburg virus or Dengue Fever virus.

The present invention also provides a method of decreasing infection in a subject comprising administering to the subject an effective amount of a composition that decreases expression or activity of a gene or a gene product set forth in Table 1, wherein the composition inhibits infection by three or more pathogens. The three or more pathogens can be selected from the viruses, bacteria, parasites and fungi set forth herein. More particularly, the three or more pathogens can be selected from the group consisting of: an HIV virus, a pox virus, a herpes virus, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

The present invention also provides a method of decreasing infection in a subject comprising administering to the subject an effective amount of a composition that decreases expression or activity of a gene or a gene product set forth in Table 1, wherein the composition inhibits infection by four or more pathogens. The four or more pathogens can be selected from the viruses, bacteria, parasites and fungi set forth herein. More particularly, the four or more pathogens can be selected from the group consisting of: a pox virus, BVDV, a herpes virus, HIV, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

The present invention also provides a method of decreasing infection in a subject comprising administering to the subject an effective amount of a composition that decreases expression or activity of a gene or a gene product set forth in Table 1, wherein the composition inhibits infection by five or more pathogens. The five or more pathogens can be selected from the viruses, bacteria, parasites and fungi set forth herein. More particularly, the five or more pathogens can be selected from the group consisting of: a pox virus, BVDV, a herpes virus, HIV, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

The present invention also provides a method of decreasing infection in a subject comprising administering to the subject an effective amount of a composition that decreases expression or activity of a gene or a gene product set forth in Table 1, wherein the composition inhibits infection by six or more pathogens. The six or more pathogens can be selected from the viruses, bacteria, parasites and fungi set forth herein. More particularly, the six or more pathogens can be selected from the group consisting of: a pox virus, BVDV, a herpes virus, HIV, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

The present invention also provides a method of decreasing infection in a subject comprising administering to the subject an effective amount of a composition that decreases expression or activity of a gene or a gene product set forth in Table 1, wherein the composition inhibits co-infection by HIV and one or more viruses, bacteria, parasites or fungi. For example, decreasing co-infection of HIV and any of the viruses, including for example any families, genus, species, or group of viruses. As a further example, co-infection of HIV and a respiratory virus is provided herein. Respiratory viruses include picornaviruses, orthomyxoviruses, paramyxoviruses, coronaviruses, and adenoviruses. More specifically, the respiratory virus can be any strain of influenza, rhinovirus, adenovirus, parainfluenza virus or RSV. Also provided is decreasing co-infection of HIV and a gastrointestinal virus. Gastrointestinal viruses include picornaviruses, filoviruses, flaviviruses, calciviruses and reoviruses. More specifically, and not to be limiting, the gastrointestinal virus can be any strain of reovirus, a Norwalk virus, an Ebola virus, a Marburg virus, a rotavirus, an enterovirus, a Dengue fever virus, a yellow fever virus, or a West Nile virus. Further provided is a method of decreasing co-infection of HIV with a pox virus, LCM, Junin virus, Machupo virus, Guanarito virus, Lassa Fever virus, hantavirus, Rift Valley Fever virus Ebola virus, Marburg virus or Dengue Fever virus. More particularly, decreasing co-infection of HIV and a hepatitis virus, such as Hepatitis A, Hepatitis B or Hepatitis C is provided. Also provided is decreasing co-infection of HIV and a herpes virus, for example, HSV-1 or HSV-2. In addition decreasing co-infection of HIV and tuberculosis is also provided. Further provided is decreasing co-infection of HIV and CMV, as well as decreasing co-infection of HIV and HPV.

As described herein, the genes set forth in Tables 1 can be involved in the pathogenesis of two or more respiratory viruses. Therefore, the present invention provides methods of treating or preventing an unspecified respiratory infection in a subject by administering a composition that decreases activity or expression of a gene involved in the pathogenesis of two or more respiratory viruses. More particularly, the present invention provides a method of decreasing an unspecified respiratory infection in a subject comprising: a) diagnosing a subject with an unspecified respiratory infection; and b) administering to the subject an effective amount of a composition that decreases expression or activity of a gene or a gene product set forth in Table 1, wherein the composition inhibits infection by two or more respiratory viruses selected from the group consisting of picornaviruses, orthomyxoviruses, paramyxoviruses, coronaviruses, or adenoviruses. As set forth above, in the methods of the present invention, the two or more respiratory viruses can be from the same family or from a different family of respiratory viruses. More specifically, the respiratory virus can be any strain of influenza, rhinovirus, adenovirus, parainfluenza virus or RSV. In this method, the composition can be a composition that inhibits infection by three or more, four or more, five or more; or six or more respiratory viruses selected from the group consisting of a picornaviruses, an orthomyxoviruses, paramyxoviruses, coronaviruses, or adenoviruses.

As described herein, the genes set forth in Tables 1 can be involved in the pathogenesis of two or more gastrointestinal viruses. Therefore, the present invention provides methods of treating or preventing an unspecified gastrointestinal infection in a subject by administering a composition that decreases activity or expression of a gene involved in the pathogenesis of two or more gastrointestinal viruses. More particularly, the present invention provides a method of decreasing an unspecified gastrointestinal infection in a subject comprising: a) diagnosing a subject with an unspecified gastrointestinal infection; and b) administering to the subject an effective amount of a composition that decreases expression or activity of a gene or a gene product set forth in Table 1, wherein the composition inhibits infection by two or more gastrointestinal viruses selected from the group consisting of a flavivirus, a filovirus, a calcivirus or a reovirus. As set forth above, in the methods of the present invention, the two or more gastrointestinal viruses can be from the same family or from a different family of gastrointestinal viruses. More particularly, and not to be limiting, the gastrointestinal virus can be any strain of reovirus, a Norwalk virus, an Ebola virus, a Marburg virus, a rotavirus, an enterovirus, a Dengue fever virus, a yellow fever virus, or a West Nile virus. In this method, the composition can be a composition that inhibits infection by three or more, four or more, five or more; or six or more gastrointestinal viruses selected from the group consisting of a flavivirus, a filovirus, a calcivirus or a reovirus.

The present invention also provides a method of preventing or decreasing an unspecified pandemic or bioterror threat in a subject comprising: a) diagnosing a subject with an unspecified pandemic or bioterrorist inflicted infection; and b) administering to the subject an effective amount of a composition that decreases expression or activity of a gene or a gene product set forth in Table 1, wherein the composition inhibits infection by two or more, three or more, four or more; or five or more viruses selected from the group consisting of a pox virus, an influenza virus, West Nile virus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus and a Dengue fever virus.

Also provided by the present invention is a method of managing secondary infections in a patient comprising administering to the subject an effective amount of a composition that decreases expression or activity of a gene or a gene product set forth in Table 1, wherein the composition can inhibit infection by HIV and one or more, two or more, three or more, four or more; or five or more secondary infections.

As set forth above, the genes set forth in Table 1 can be involved in the pathogenesis of three or more pathogens. Therefore, the present invention provides methods of treating or preventing an unspecified infection by administering a composition that decreases the activity or expression of a gene that is involved in the pathogenesis of three or more pathogens. Therefore, the present invention provides a method of decreasing infection in a subject comprising: a) diagnosing a subject with an unspecified infection and; b) administering a composition that decreases the expression or activity of a gene or gene product set forth in Table 1, wherein the composition decreases infection by three or more pathogens. More specifically, the three or more pathogens can be selected from the group consisting of: a pox virus, BVDV, a herpes virus, HIV, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

The infection can be a viral infection, a parasitic infection, a bacterial infection or a fungal infection, to name a few. As utilized herein, “an unspecified infection” is an infection that presents symptoms associated with an infection, but is not identified as specific infection. One of skill in the art, for example, a physician, a nurse, a physician's assistant, a medic or any other health practitioner would know how to diagnose the symptoms of infection even though the actual pathogen may not be known. For example, the patient can present with one or more symptoms, including, but not limited to, a fever, fatigue, lesions, weight loss, inflammation, a rash, pain (for example, muscle ache, headache, ear ache, joint pain, etc.), urinary difficulties, respiratory symptoms (for example, coughing, bronchitis, lung failure, breathing difficulties, bronchiolitis, airway obstruction, wheezing, runny nose, sinusitis, congestion, etc.), gastrointestinal symptoms (for example, nausea, diarrhea, vomiting, dehydration, abdominal pain, intestinal cramps, rectal bleeding, etc.), This can occur in the event of a bioterrorist attack or a pandemic. In this event, one of skill in the art would know to administer a composition that inhibits infection by decreasing the expression or activity of a gene or gene product set forth in Table 1 that is involved in the pathogenesis of several pathogens. Similarly, if there is a threat of an unspecified infection, for example, a threat of a bioterrorist attack, a composition that decreases the expression or activity of a gene or gene product set forth in Table 1 can be administered prophylactically to a subject to prevent an unspecified infection in a subject.

By “treat,” “treating,” or “treatment” is meant a method of reducing the effects of an existing infection. Treatment can also refer to a method of reducing the disease or condition itself rather than just the symptoms. The treatment can be any reduction from native levels and can be, but is not limited to, the complete ablation of the disease or the symptoms of the disease. Treatment can range from a positive change in a symptom or symptoms of viral infection to complete amelioration of the viral infection as detected by art-known techniques. For example, a disclosed method is considered to be a treatment if there is about a 10% reduction in one or more symptoms of the disease in a subject with the disease when compared to native levels in the same subject or control subjects. Thus, the reduction can be about a 10, 20, 30, 40, 50, 60, 70, 80, 90, 100%, or any amount of reduction in between as compared to native or control levels.

The methods of the present invention can also result in a decrease in the amount of time that it normally takes to see improvement in a subject. For example, a decrease in infection can be a decrease of hours, a day, two days, three days, four days, five days, six days, seven days, eight days, nine days, ten days, eleven days, twelve days, thirteen days, fourteen days, fifteen days or any time in between that it takes to see improvement in the symptoms, viral load or any other parameter utilized to measure improvement in a subject. For example, if it normally takes 7 days to see improvement in a subject not taking the composition, and after administration of the composition, improvement is seen at 6 days, the composition is effective in decreasing infection. This example is not meant to be limiting as one of skill in the art would know that the time for improvement will vary depending on the infection.

As utilized herein, by “prevent,” “preventing,” or “prevention” is meant a method of precluding, delaying, averting, obviating, forestalling, stopping, or hindering the onset, incidence, severity, or recurrence of infection. For example, the disclosed method is considered to be a prevention if there is about a 10% reduction in onset, incidence, severity, or recurrence of infection, or symptoms of infection (e.g., inflammation, fever, lesions, weight loss, etc.) in a subject exposed to an infection when compared to control subjects exposed to an infection that did not receive a composition for decreasing infection. Thus, the reduction in onset, incidence, severity, or recurrence of infection can be about a 10, 20, 30, 40, 50, 60, 70, 80, 90, 100%, or any amount of reduction in between as compared to control subjects. For example, and not to be limiting, if about 10% of the subjects in a population do not become infected as compared to subjects that did not receive preventive treatment, this is considered prevention.

Also provided is a method of decreasing infection in a subject comprising: a) administering a composition that decreases the expression or activity of a gene or gene product set forth in Table 1 in a subject with an unspecified infection; b) diagnosing the type of infection in the subject and; c) administering a composition that decreases the expression or activity of a gene or a gene product set forth in Table 1 for the diagnosed infection. Further provided is a method of treating viral infection comprising: a) diagnosing a subject with a viral infection; and b) removing a drug from the subject that decreases the expression or activity of a gene or gene product set forth in Table 1, if the viral infection is not a viral infection that is inhibited by a composition that decreases the expression or activity of a gene or gene product set forth in Table 1. As mentioned above, upon recognizing that a subject has an infection or the symptoms of an infection, for example, in the case of a bioterrorist attack or a pandemic, given that a gene or gene product set forth in Table 1 can be involved in the pathogenesis of several pathogens, a practitioner can prescribe or administer a composition that decreases the expression or activity of the gene or gene product. After administration, the practitioner, who can be the same practitioner or a different practitioner, can diagnose the type of infection in a subject. This diagnosis can be a differential diagnosis where the practitioner distinguishes between infections by comparing signs or symptoms and eliminates certain types of infection before arriving at the diagnosis for a specific infection, or a diagnosis based on a test that is specific for a particular infection. Once a specific infection is diagnosed, if the gene or gene product is involved in the pathogenesis of this infection, the practitioner can prescribe or administer a composition that decreases the expression or activity of that gene or gene product. This can be the same composition administered prior to diagnosis of the specific infection or a different composition that decreases expression or activity.

Also provided is a method of preventing infection in a subject comprising administering to a subject susceptible to an unspecified infection a composition that decreases the expression or activity of a gene or gene product set forth in Table 1. The composition can be administered in response to a lethal outbreak of an infection. For example, the infection can be a pandemic or a bioterrorist created infection. If there is a threat of an unspecified infection, such as a viral infection, a bacterial infection, a parasitic infection or an infection by a chimeric pathogenic agent, to name a few, a composition can be administered prophylactically to a subject to prevent an unspecified infection in a subject. The threat can also come in the form of a toxin. One of skill in the art would know to administer a composition that inhibits infection by decreasing the expression or activity of any gene or gene product set forth in Table 1 that is involved in the pathogenesis of two or more, three ore more, four or more; or five or more pathogens.

Such prophylactic use can decrease the number of people in a population that are infected, thus preventing further spread of a pandemic or decreasing the effects of a bioterrorist attack.

In the methods of the present invention, the composition can comprise one or more of, a chemical, a compound, a small molecule, an inorganic molecule, an aptamer, an organic molecule, a drug, a protein, a cDNA, a peptide, an antibody, a morpholino, a triple helix molecule, an siRNA, an shRNAs, an miRNA, an antisense nucleic acid or a ribozyme that decreases the expression or activity of a gene or gene product set forth in Table 1. The composition can be administered before or after infection. The decrease in infection in a subject need not be complete as this decrease can be a 10%, 20%, 30%, 40%, 50%, 60%, 70%, 80%, 90%, 100% or any other percentage decrease in between as long as a decrease occurs. This decrease can be correlated with amelioration of symptoms associated with infection. These compositions can be administered to a subject alone or in combination with other therapeutic agents described herein, such as anti-viral compounds, antibacterial agents, antifungal agents, antiparasitic agents, anti-inflammatory agents, anti-cancer agents, etc. Examples of viral infections, bacterial infections, fungal infections parasitic infections are set forth above. The compounds set forth herein or identified by the screening methods set forth herein can be administered to a subject to decrease infection by any pathogen or infectious agent set forth herein. Any of the compounds set forth herein or identified by the screening methods of the present invention can also be administered to a subject to decrease infection by any pathogen, now known or later discovered in which a gene or gene product set forth in Table 1 is involved.

Various delivery systems for administering the therapies disclosed herein are known, and include encapsulation in liposomes, microparticles, microcapsules, expression by recombinant cells, receptor-mediated endocytosis (Wu and Wu, J. Biol. Chem. 1987, 262:4429-32), and construction of therapeutic nucleic acids as part of a retroviral or other vector. Methods of introduction include, but are not limited to, mucosal, topical, intradermal, intrathecal, intranasal, intratracheal, via nebulizer, via inhalation, intramuscular, otic delivery (ear), eye delivery (for example, eye drops), intraperitoneal, vaginal, rectal, intravenous, subcutaneous, intranasal, and oral routes. The compounds can be administered by any convenient route, for example by infusion or bolus injection, by absorption through epithelial or mucocutaneous linings (for example, oral mucosa, rectal, vaginal and intestinal mucosa, etc.) and can be administered together with other biologically active agents. Administration can be systemic or local. Pharmaceutical compositions can be delivered locally to the area in need of treatment, for example by topical application or local injection.

Pharmaceutical compositions are disclosed that include a therapeutically effective amount of a RNA, DNA, antisense molecule, ribozyme, siRNA, shRNA molecule, miRNA molecule, aptamer, drug, protein, small molecule, peptide inorganic molecule, organic molecule, antibody or other therapeutic agent, alone or with a pharmaceutically acceptable carrier. Furthermore, the pharmaceutical compositions or methods of treatment can be administered in combination with (such as before, during, or following) other therapeutic treatments, such as other antiviral agents, antibacterial agents, antifungal agents and antiparasitic agents.

For all of the administration methods disclosed herein, each method can optionally comprise the step of diagnosing a subject with an infection or diagnosing a subject in need of prophylaxis or prevention of infection.

### Delivery Systems

The pharmaceutically acceptable carriers useful herein are conventional. Remington's Pharmaceutical Sciences, by Martin, Mack Publishing Co., Easton, Pa., 15th Edition (1975), describes compositions and formulations suitable for pharmaceutical delivery of the therapeutic agents herein disclosed. In general, the nature of the carrier will depend on the mode of administration being employed. For instance, parenteral formulations usually include injectable fluids that include pharmaceutically and physiologically acceptable fluids such as water, physiological saline, balanced salt solutions, aqueous dextrose, sesame oil, glycerol, ethanol, combinations thereof, or the like, as a vehicle. The carrier and composition can be sterile, and the formulation suits the mode of administration. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The composition can be a liquid solution, suspension, emulsion, tablet, pill, capsule, sustained release formulation, or powder. For solid compositions (for example powder, pill, tablet, or capsule forms), conventional non-toxic solid carriers can include, for example, pharmaceutical grades of mannitol, lactose, starch, sodium saccharine, cellulose, magnesium carbonate, or magnesium stearate. The composition can be formulated as a suppository, with traditional binders and carriers such as triglycerides.

Embodiments of the disclosure including medicaments can be prepared with conventional pharmaceutically acceptable carriers, adjuvants and counterions as would be known to those of skill in the art.

The amount of therapeutic agent effective in decreasing or inhibiting infection can depend on the nature of the pathogen and its associated disorder or condition, and can be determined by standard clinical techniques. Therefore, these amounts will vary depending on the type of virus, bacteria, fungus, parasite or other pathogen. For example, the dosage can be anywhere from 0.01 mg/kg to 100 mg/kg. Multiple dosages can also be administered depending on the type of pathogen, and the subject's condition. In addition, in vitro assays can be employed to identify optimal dosage ranges. The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems.

The disclosure also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

In an example in which a nucleic acid is employed to reduce infection, such as an antisense or siRNA molecule, the nucleic acid can be delivered intracellularly (for example by expression from a nucleic acid vector or by receptor-mediated mechanisms), or by an appropriate nucleic acid expression vector which is administered so that it becomes intracellular, for example by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents, or by administering it in linkage to a homeobox-like peptide which is known to enter the nucleus (for example Joliot et al., Proc. Natl. Acad. Sci. USA 1991, 88:1864-8). siRNA carriers also include, polyethylene glycol (PEG), PEG-liposomes, branched carriers composed of histidine and lysine (HK polymers), chitosan-thiamine pyrophosphate carriers, surfactants (for example, Survanta and Infasurf), nanochitosan carriers, and D5W solution. The present disclosure includes all forms of nucleic acid delivery, including synthetic oligos, naked DNA, plasmid and viral delivery, integrated into the genome or not.

As mentioned above, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells a nucleic acid, for example an antisense molecule or siRNA. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), and pseudotyped retroviral vectors (Agrawal et al., Exper. Hematol. 24:738-747, 1996). Other nonpathogenic vector systems such as the foamy virus vector can also be utilized (Park et al. “Inhibition of simian immunodeficiency virus by foamy virus vectors expressing siRNAs.” Virology. 2005 Sep. 20). It is also possible to deliver short hairpin RNAs (shRNAs) via vector delivery systems in order to inhibit gene expression (See Pichler et al. “In vivo RNA interference-mediated ablation of MDR1 P-glycoprotein.” Clin Cancer Res. 2005 Jun. 15; 11(12):4487-94; Lee et al. “Specific inhibition of HIV-1 replication by short hairpin RNAs targeting human cyclin T1 without inducing apoptosis.” FEBS Lett. 2005 Jun. 6; 579(14):3100-6.).

Physical transduction techniques can also be used, such as liposome delivery and receptor-mediated and other endocytosis mechanisms (see, for example, Schwartzenberger et al., Blood 87:472-478, 1996) to name a few examples. This invention can be used in conjunction with any of these or other commonly used gene transfer methods.

### Transgenic Cells and Non-Human Mammals

The present invention also provides a non-human transgenic mammal comprising a functional deletion of a gene set forth in Table 1, wherein the mammal has decreased susceptibility to infection by a pathogen, such as a virus, a bacterium, a fungus or a parasite. Exemplary transgenic non-human mammals include, but are not limited to, ferrets, fish, guinea pigs, chinchilla, mice, monkeys, rabbits, rats, chickens, cows, and pigs. Such knock-out animals are useful for reducing the transmission of viruses from animals to humans and for further validating a target. In the transgenic animals of the present invention one or both alleles of a gene set forth in Table 1 can be functionally deleted.

By “decreased susceptibility” is meant that the animal is less susceptible to infection or experiences decreased infection by a pathogen as compared to an animal that does not have one or both alleles of a gene set forth in Table 1 functionally deleted. The animal does not have to be completely resistant to the pathogen. For example, the animal can be 10%, 20%, 30%, 40%, 50%, 60%, 70%, 80%, 90%, 100% or any percentage in between less susceptible to infection by a pathogen as compared to an animal that does not have a functional deletion of a gene set forth in Table 1. Furthermore, decreasing infection or decreasing susceptibility to infection includes decreasing entry, replication, pathogenesis, insertion, lysis, or other steps in the replication strategy of a virus or other pathogen into a cell or subject, or combinations thereof.

Therefore, the present invention provides a non-human transgenic mammal comprising a functional deletion of a gene set forth in Table 1, wherein the mammal has decreased susceptibility to infection by a pathogen, such as a virus, a bacterium, a parasite or a fungus. A functional deletion is a mutation, partial or complete deletion, insertion, or other variation made to a gene sequence that inhibits production of the gene product or renders a gene product that is not completely functional or non-functional. Functional deletions can be made by insertional mutagenesis (for example via insertion of a transposon or insertional vector), by site directed mutagenesis, via chemical mutagenesis, via radiation or any other method now known or developed in the future that results in a transgenic animal with a functional deletion of a gene set forth in Table 1.

Alternatively, a nucleic acid sequence such as siRNA, a morpholino or another agent that interferes with a gene set forth in Table 1 can be delivered. The expression of the sequence used to knock-out or functionally delete the desired gene can be regulated by an appropriate promoter sequence. For example, constitutive promoters can be used to ensure that the functionally deleted gene is not expressed by the animal. In contrast, an inducible promoter can be used to control when the transgenic animal does or does not express the gene of interest. Exemplary inducible promoters include tissue-specific promoters and promoters responsive or unresponsive to a particular stimulus (such as light, oxygen, chemical concentration, such as a tetracycline inducible promoter).

The transgenic animals of the present invention that comprise a functionally deleted a gene set forth in Table 1 can be examined during exposure to various pathogens. Comparison data can provide insight into the life cycles of pathogens. Moreover, knock-out animals or functionally deleted (such as birds or pigs) that are otherwise susceptible to an infection (for example influenza) can be made to resist infection, conferred by disruption of the gene. If disruption of the gene in the transgenic animal results in an increased resistance to infection, these transgenic animals can be bred to establish flocks or herds that are less susceptible to infection.

Transgenic animals, including methods of making and using transgenic animals, are described in various patents and publications, such as WO 01/43540; WO 02/19811; U.S. Pub. Nos: 2001-0044937 and 2002-0066117; and U.S. Pat. Nos. 5,859,308; 6,281,408; and 6,376,743; and the references cited therein.

The transgenic animals of this invention also include conditional gene knockdown animals produced, for example, by utilizing the SIRIUS-Cre system that combines siRNA for specific gene-knockdown, Cre-loxP for tissue-specific expression and tetracycline-on for inducible expression. These animals can be generated by mating two parental lines that contain a specific siRNA of interest gene and tissue-specific recombinase under tetracycline control. See Chang et al. “Using siRNA Technique to Generate Transgenic Animals with Spatiotemporal and Conditional Gene Knockdown.” American Journal of Pathology 165: 1535-1541 (2004) which is hereby incorporated in its entirety by this reference regarding production of conditional gene knockdown animals.

The present invention also provides cells including an altered or disrupted gene set forth in Table 1 that are resistant to infection by a pathogen. These cells can be in vitro, ex vivo or in vivo cells and can have one or both alleles altered. These cells can also be obtained from the transgenic animals of the present invention. Such cells therefore include cells having decreased susceptibility to a virus or any of the other pathogens described herein, including bacteria, parasites and fungi.

Since the genes set forth herein are involved in viral infection, also provided herein are methods of overexpressing any of the genes set forth in Table 1 in host cells. Overexpression of these genes can provide cells that increase the amount of virus produced by the cell, thus allowing more efficient production of viruses. Also provided is the overexpression of the genes set forth herein in avian eggs, for example, in chicken eggs.

Methods of screening agents, such as a chemical, a compound, a small or large molecule, an organic molecule, an inorganic molecule, a peptide, a drug, a protein, a cDNA, an antibody, a morpholino, a triple helix molecule, an siRNA, an shRNAs, an miRNA, an antisense nucleic acid or a ribozyme set forth using the transgenic animals described herein are also provided.

### Screening for Resistance to Infection

Also provided herein are methods of screening host subjects for resistance to infection by characterizing a nucleotide sequence or amino acid sequence of a host gene set forth in Table 1. The nucleic acid or amino acid sequence of a subject can be isolated, sequenced, and compared to the wildtype sequence of a gene set forth in Table 1. The greater the similarity between that subject's nucleic acid sequence or amino acid sequence and the wildtype sequence, the more susceptible that person is to infection, while a decrease in similarity between that subject's nucleic acid sequence or amino acid sequence and the wildtype sequence, the more resistant that subject can be to infection. Such screens can be performed for any gene set forth in Table lfor any species.

Assessing the genetic characteristics of a population can provide information about the susceptibility or resistance of that population to viral infection. For example, polymorphic analysis of alleles in a particular human population, such as the population of a particular city or geographic area, can indicate how susceptible that population is to infection. A higher percentage of alleles substantially similar to a wildtype gene set forth in Table 1 can indicate that the population is more susceptible to infection, while a large number of polymorphic alleles that are substantially different than a wildtype gene sequence can indicate that a population is more resistant to infection. Such information can be used, for example, in making public health decisions about vaccinating susceptible populations.

The present invention also provides a method of screening a cell for a variant form of a gene set forth in Table 1. A variant can be a gene with a functional deletion, mutation or alteration in the gene such that the amount or activity of the gene product is altered. These cells containing a variant form of a gene can be contacted with a pathogen to determine if cells comprising a naturally occurring variant of a gene set forth in Table 1 differs in their resistance to infection. For example, cells from an animal, for example, a chicken, can be screened for a variant form of a gene set forth in Table 1. If a naturally occurring variant is found and chickens possessing a variant form of the gene in their genome are less susceptible to infection, these chickens can be selectively bred to establish flocks that are resistant to infection. By utilizing these methods, flocks of chickens that are resistant to avian flu or other pathogens can be established. Similarly, other animals can be screened for a variant form of a gene set forth in Table 1. If a naturally occurring variant is found and animals possessing a variant form of the gene in their genome are less susceptible to infection, these animals can be selectively bred to establish populations that are resistant to infection. These animals include, but are not limited to, cats, dogs, livestock (for example, cattle, horses, pigs, sheep, goats, etc.), laboratory animals (for example, mouse, monkey, rabbit, rat, gerbil, guinea pig, etc.) and avian species (for example, flocks of chickens, geese. turkeys, ducks, pheasants, pigeons, doves etc.). Therefore, the present application provides populations of animals that comprise a naturally occurring variant of a gene set forth in Table 1 that results in decreased susceptibility to viral infection, thus providing populations of animals that are less susceptible to viral infection. Similarly, if a naturally occurring variant is found and animals possessing a variant form of the gene in their genome are less susceptible to bacterial, parasitic or fungal infection, these animals can be selectively bred to establish populations that are resistant to bacterial, parasitic or fungal infection.

### Screening Methods

The present invention provides a method of identifying a compound that binds to a gene product set forth in Table 1 and can decrease infection of a cell by a pathogen comprising: a) contacting a compound with a gene product set forth in Table 1; b) detecting binding of the compound to the gene product; and c) associating the binding with a decrease in infection by the pathogen.

The present invention provides a method of identifying an agent that decreases infection of a cell by a pathogen comprising: a) administering the agent to a cell containing a cellular gene encoding a gene product set forth in Table 1; and b) detecting the level and/or activity of the gene product produced by the cellular gene, a decrease or elimination of the gene product and/or gene product activity indicating an agent with antipathogenic activity.

Also provided is a method of identifying an agent that decreases infection in a cell by a pathogen comprising: a) administering the agent to a cell containing a cellular gene encoding a gene product set forth in Table 1; b) contacting the cell with a pathogen; and c) determining the level of infection, a decrease or elimination of infection indicating that the agent is an agent that decreases infection.

The present invention also provides a method of identifying a compound that binds to a gene product set forth in Table 1 and can decrease infection by three or more pathogens comprising: a) contacting a compound with a gene product set forth in Table 1; b) detecting binding of the compound to the gene product; and c) associating binding with a decrease in infection by three or more pathogens. This method can further comprise optimizing a compound that binds the gene product in an assay that determines the functional ability to decrease infection by three or more pathogens. This method can be cell based or an in vivo assay. The three or more pathogens can be any three or more pathogens set forth herein. For example, the three or more pathogens can be respiratory pathogens selected from the group consisting of picornaviruses, orthomyxoviruses, paramyxoviruses, coronaviruses or adenoviruses. In another example, the three or more pathogens can be gastrointestinal pathogens selected from filoviruses, flaviviruses, calciviruses and reoviruses. The three or more pathogens can also be a combination of respiratory and gastrointestinal viruses. In another example, the three or more pathogens can be selected from the group consisting of: an HIV virus, a pox virus, a herpes virus, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus. The cell population used in the method can be the same cell population for each pathogen or can be different cell populations. Typically, the agent would be administered to a different cell population for each pathogen assayed. For example, and not to be limiting, if the pathogens are viruses, a cell population is contacted with the agent and a first virus, another cell population is contacted with the agent and second virus, a third cell population is contacted with the agent and a third virus etc. in order to determine whether the agent inhibits infection by three or more viruses. Since the cell type will vary depending on whether or not a given virus can infect the cell, one of skill in the art would know how to pair the cell type with the virus in order to perform the assay.

This method can further comprise measuring the level of expression and/or activity of the gene product set forth in Table 1. This method can further comprise associating the level of infection with the level of expression and/or activity a gene product set forth in Table 1. In the screening methods disclosed herein, the level of infection can be measured, for example, by measuring viral replication.

In the methods of the present invention, if the agent has previously been identified as an agent that decreases or inhibits the level and/or activity of a gene product set forth in Table 1, this can indicate a decrease in infection. A decrease in infection as compared to infection in a cell that was not contacted with the agent known to decrease or inhibit the level and/or activity of the gene product can be sufficient to identify the agent as an agent that decreases or inhibits infection.

The methods described above can be utilized to identify any agent with an activity that decreases infection, prevents infection or promotes cellular survival after infection with a pathogen(s). Therefore, the cell can be contacted with a pathogen before, or after being contacted with the agent. The cell can also be contacted concurrently with the pathogen and the agent. The agents identified utilizing these methods can be used to inhibit infection in cells either in vitro, ex vivo or in vivo.

In the methods of the present invention any cell that can be infected with a pathogen can be utilized. The cell can be prokaryotic or eukaryotic, such as a cell from an insect, fish, crustacean, mammal, bird, reptile, yeast or a bacterium, such as E. coli. The cell can be part of an organism, or part of a cell culture, such as a culture of mammalian cells or a bacterial culture. The cell can also be in a nonhuman subject thus providing in vivo screening of agents that decrease infection by a pathogen. Cells susceptible to infection are well known and can be selected based on the pathogen of interest.

The test agents or compounds used in the methods described herein can be, but are not limited to, chemicals, small molecules, inorganic molecules, organic molecules, drugs, proteins, cDNAs, large molecules, antibodies, morpholinos, triple helix molecule, peptides, siRNAs, shRNAs, miRNAs, antisense RNAs, ribozymes or any other compound. The compound can be random or from a library optimized to bind to a gene or gene product set forth in Table 1. Drug libraries optimized for the proteins in the class of proteins provided herein can also be screened or tested for binding or activity. Compositions identified with the disclosed approaches can be used as lead compositions to identify other compositions having even greater antipathogenic activity. For example, chemical analogs of identified chemical entities, or variants, fragments or fusions of peptide agents, can be tested for their ability to decrease infection using the disclosed assays. Candidate agents can also be tested for safety in animals and then used for clinical trials in animals or humans.

In the methods described herein, once the cell containing a cellular gene encoding a gene product set forth in Table 1 has been contacted with an agent, the level of infection can be assessed by measuring an antigen or other product associated with a particular infection. For example, the level of viral infection can be measured by real-time quantitative reverse transcription-polymerase chain reaction (RT-PCR) assay (See for example, Payungporn et al. “Single step multiplex real-time RT-PCR for H5N1 influenza A virus detection.” J Virol Methods. Sep. 22, 2005; Landolt et al. “Use of real-time reverse transcriptase polymerase chain reaction assay and cell culture methods for detection of swine influenza A viruses” Am J Vet Res. 2005 January; 66(1):119-24). If there is a decrease in infection then the composition is an effective agent that decreases infection. This decrease does not have to be complete as the decrease can be a 10%, 20%, 30%, 40%, 50%, 60%. 70%, 80%, 90%, 100% decrease or any percentage decrease in between.

In the methods set forth herein, the level of the gene product can be measured by any standard means, such as by detection with an antibody specific for the protein. The nucleic acids set forth herein and fragments thereof can be utilized as primers to amplify nucleic acid sequences, such as a gene transcript of a gene set forth in Table 1 by standard amplification techniques. For example, expression of a gene transcript can be quantified by real time PCR using RNA isolated from cells. A variety of PCR techniques are familiar to those skilled in the art. For a review of PCR technology, see White (1997) and the publication entitled “PCR Methods and Applications” (1991, Cold Spring Harbor Laboratory Press), which is incorporated herein by reference in its entirety for amplification methods. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188. Each of these publications is incorporated herein by reference in its entirety for PCR methods. One of skill in the art would know how to design and synthesize primers that amplify any of the nucleic acid sequences set forth herein or a fragment thereof.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g., ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified fragment, can be analyzed by one of a number of methods known in the art. The nucleic acid can be sequenced by dideoxy or other methods. Hybridization with the sequence can also be used to determine its presence, by Southern blots, dot blots, etc.

In the methods of the present invention, the level of gene product can be compared to the level of the gene product in a control cell not contacted with the compound. The level of gene product can be compared to the level of the gene product in the same cell prior to addition of the compound. Activity or function, can be measured by any standard means, such as by enzymatic assays that measure the conversion of a substrate to a product or binding assays that measure the binding of a gene product set forth in Table 1 to another protein, for example.

Moreover, the regulatory region of a gene set forth in Table 1 can be functionally linked to a reporter gene and compounds can be screened for inhibition of reporter gene expression. Such regulatory regions can be isolated from genomic sequences and identified by any characteristics observed that are characteristic for regulatory regions of the species and by their relation to the start codon for the coding region of the gene. As used herein, a reporter gene encodes a reporter protein. A reporter protein is any protein that can be specifically detected when expressed. Reporter proteins are useful for detecting or quantitating expression from expression sequences. Many reporter proteins are known to one of skill in the art. These include, but are not limited to, β-galactosidase, luciferase, and alkaline phosphatase that produce specific detectable products. Fluorescent reporter proteins can also be used, such as green fluorescent protein (GFP), cyan fluorescent protein (CFP), red fluorescent protein (RFP) and yellow fluorescent protein (YFP).

Viral infection can also be measured via cell based assays. Briefly, cells (20,000 to 2,500,000) are infected with the desired pathogen, and the incubation continued for 3-7 days. The antiviral agent can be applied to the cells before, during, or after infection with the pathogen. The amount of virus and agent administered can be determined by skilled practitioners. In some examples, several different doses of the potential therapeutic agent can be administered, to identify optimal dose ranges. Following transfection, assays are conducted to determine the resistance of the cells to infection by various agents.

For example, if analyzing viral infection, the presence of a viral antigen can be determined by using antibody specific for the viral protein then detecting the antibody. In one example, the antibody that specifically binds to the viral protein is labeled, for example with a detectable marker such as a fluorophore. In another example, the antibody is detected by using a secondary antibody containing a label. The presence of bound antibody is then detected, for example using microscopy, flow cytometry and ELISA. Similar methods can be used to monitor bacterial, protozoal, or fungal infection (except that the antibody would recognize a bacterial, protozoal, or fungal protein, respectively).

Alternatively, or in addition, the ability of the cells to survive viral infection is determined, for example, by performing a cell viability assay, such as trypan blue exclusion. Plaque assays can be utilized as well.

The amount of protein in a cell, can be determined by methods standard in the art for quantitating proteins in a cell, such as Western blotting, ELISA, ELISPOT, immunoprecipitation, immunofluorescence (e.g., FACS), immunohistochemistry, immunocytochemistry, etc., as well as any other method now known or later developed for quantitating protein in or produced by a cell.

The amount of a nucleic acid in a cell can be determined by methods standard in the art for quantitating nucleic acid in a cell, such as in situ hybridization, quantitative PCR, RT-PCR, Taqman assay, Northern blotting, ELISPOT, dot blotting, etc., as well as any other method now known or later developed for quantitating the amount of a nucleic acid in a cell.

The ability of an antiviral agent to prevent or decrease infection by a virus, for example, any of the viruses listed above, can be assessed in an animal model. Several animal models for viral infection are known in the art. For example, mouse HIV models are disclosed in Sutton et al. (Res. Initiat Treat. Action, 8:22-4, 2003) and Pincus et al. (AIDS Res. Hum. Retroviruses 19:901-8, 2003); guinea pig models for Ebola infection are disclosed in Parren et al. (J. Virol. 76:6408-12, 2002) and Xu et al. (Nat. Med. 4:37-42, 1998); cynomolgus monkey (Macaca fascicularis) models for influenza infection are disclosed in Kuiken et al. (Vet. Pathol. 40:304-10, 2003); mouse models for herpes are disclosed in Wu et al. (Cell Host Microbe 22:5(1):84-94. 2009); pox models are disclosed in Smee et al. (Nucleosides Nucleotides Nucleic Acids 23(1-2):375-83, 2004) and in Bray et al. (J. Infect. Dis. 181(1):10-19); and Franciscella tularensis models are disclosed in Klimpel et al. (Vaccine 26(52): 6874-82, 2008).

Other animal models for influenza infection are also available. These include, but are not limited to, a cotton rat model disclosed by Ottolini et al. (J. Gen. Virol., 86(Pt 10): 2823-30, 2005), as well as ferret and mouse models disclosed by Maines et al. (J. Virol. 79(18):11788-11800, 2005). One of skill in the art would know how to select an animal model for assessing the in vivo activity of an agent for its ability to decrease infection by viruses, bacteria, fungi and parasites.

Such animal models can also be used to test agents for an ability to ameliorate symptoms associated with viral infection. In addition, such animal models can be used to determine the LD₅₀ and the ED₅₀ in animal subjects, and such data can be used to determine the in vivo efficacy of potential agents. Animal models can also be used to assess antibacterial, antifungal and antiparasitic agents.

Animals of any species, including, but not limited to, birds, ferrets, cats, mice, rats, rabbits, fish (for example, zebrafish) guinea pigs, pigs, micro-pigs, goats, and non-human primates, e.g., baboons, monkeys, and chimpanzees, can be used to generate an animal model of viral infection, bacterial infection, fungal infection or parasitic infection if needed.

For example, for a model of viral infection, the appropriate animal is inoculated with the desired virus, in the presence or absence of the antiviral agent. The amount of virus and agent administered can be determined by skilled practitioners. In some examples, several different doses of the potential therapeutic agent (for example, an antiviral agent) can be administered to different test subjects, to identify optimal dose ranges. The therapeutic agent can be administered before, during, or after infection with the virus. Subsequent to the treatment, animals are observed for the development of the appropriate viral infection and symptoms associated therewith. A decrease in the development of the appropriate viral infection, or symptoms associated therewith, in the presence of the agent provides evidence that the agent is a therapeutic agent that can be used to decrease or even inhibit viral infection in a subject. For example, a virus can be tested which is lethal to the animal and survival is assessed. In other examples, the weight of the animal or viral titer in the animal can be measured. Similar models and approaches can be used for bacterial, fungal and parasitic infections.

In the methods of the present invention, the level of infection can be associated with the level of gene expression and/or activity, such that a decrease or elimination of infection associated with a decrease or elimination of gene expression and/or activity indicates that the agent is effective against the pathogen. For example, the level of infection can be measured in a cell after administration of siRNA that is known to inhibit a gene product set forth in Table 1. If there is a decrease in infection then the siRNA is an effective agent that decreases infection. This decrease does not have to be complete as the decrease can be a 10%, 20%, 30%, 40%, 50%, 60%. 70%, 80%, 90%, 100% decrease or any percentage decrease in between. In the event that the compound is not known to decrease expression and/or activity of a gene product set forth in Table 1, the level of expression and/or activity of can be measured utilizing the methods set forth above and associated with the level of infection. By correlating a decrease in expression and/or activity with a decrease in infection, one of skill in the art can confirm that a decrease in infection is effected by a decrease in expression and/or activity of a gene or gene product set forth in Table 1. Similarly, the level of infection can be measured in a cell, utilizing the methods set forth above and known in the art, after administration of a chemical, small molecule, drug, protein, cDNA, antibody, aptamer, shRNA, miRNA, morpholino, antisense RNA, ribozyme or any other compound. If there is a decrease in infection, then the chemical, small molecule, drug, protein, cDNA, antibody, shRNA, miRNA, morpholino, antisense RNA, ribozyme or any other compound is an effective antpathogenic agent.

The genes and nucleic acids of the invention can also be used in polynucleotide arrays. Polynucleotide arrays provide a high throughput technique that can assay a large number of polynucleotide sequences in a single sample. This technology can be used, for example, to identify samples with reduced expression of as compared to a control sample. This technology can also be utilized to determine the effects of reduced expression of a gene set forth in Table 1 on other genes. In this way, one of skill in the art can identify genes that are upregulated or downregulated upon reducing expression of a gene set forth in Table 1. Similarly, one of skill in the art can identify genes that are upregulated or down-regulated upon increased expression of a gene set forth in Table 1. This allows identification of other genes that are upregulated or downregulated upon modulation of expression that can be targets for therapy, such as antiviral therapy, antibacterial therapy, antiparasitic therapy or antifungal therapy.

To create arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides selected from nucleotide sequences set forth under GenBank Accession Nos. herein and other nucleic acid sequences that would be selected by one of skill in the art depending on what genes, in addition to one ore more of the genes set forth in Table 1, 2, 3 or 4 are being analyzed.

The array can also be a microarray that includes probes to different polymorphic alleles of these genes. A polymorphism exists when two or more versions of a nucleic acid sequence exist within a population of subjects. For example, a polymorphic nucleic acid can be one where the most common allele has a frequency of 99% or less. Different alleles can be identified according to differences in nucleic acid sequences, and genetic variations occurring in more than 1% of a population (which is the commonly accepted frequency for defining polymorphism) are useful polymorphisms for certain applications. The allelic frequency (the proportion of all allele nucleic acids within a population that are of a specified type) can be determined by directly counting or estimating the number and type of alleles within a population. Polymorphisms and methods of determining allelic frequencies are discussed in Hartl, D. L. and Clark, A. G., Principles of Population Genetics, Third Edition (Sinauer Associates, Inc., Sunderland Mass., 1997), particularly in chapters 1 and 2.

These microarrays can be utilized to detect polymorphic alleles in samples from subjects. Such alleles may indicate that a subject is more susceptible to infection or less susceptible to infection. For example, microarrays can be utilized to detect polymorphic versions of genes set forth in Table 1 that result in decreased gene expression and/or decreased activity of the gene product to identify subjects that are less susceptible to viral infection. In addition, the existence of an allele associated with decreased expression in a healthy individual can be used to determine which genes are likely to have the least side effects if the gene product is inhibited or bound or may be selected for in commercial animals and bred into the population.

The substrate can be any substrate to which polynucleotide probes can be attached, including but not limited to glass, nitrocellulose, silicon, and nylon. Polynucleotide probes can be bound to the substrate by either covalent bonds or by non-specific interactions, such as hydrophobic interactions. Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734. Commercially available polynucleotide arrays, such as Affymetrix GeneChip™, can also be used. Use of the GeneChip™ to detect gene expression is described, for example, in Lockhart et al., Nature Biotechnology 14:1675 (1996); Chee et al., Science 274:610 (1996); Hacia et al., Nature Genetics 14:441, 1996; and Kozal et al., Nature Medicine 2:753, 1996.

The present invention also provides a method of identifying an agent that can decrease infection by three or more pathogens comprising: a) administering the agent to three or more cell populations containing a cellular gene encoding a gene product set forth in Table 1; b) contacting the three or more cell populations with a pathogen selected wherein each population is contacted with a different pathogen; and c) determining the level of infection, a decrease or elimination of infection by three or more pathogens indicating that the agent is an agent that decreases infection by three or more pathogens. In the screening methods set forth herein, the three or more pathogens can be three or more respiratory viruses selected from the one or more families from group consisting of: picornaviruses, an orthomyxoviruses, a paramyxoviruses, a coronaviruses, or an adenoviruses. The three or more pathogens can be three or more gastrointestinal viruses selected from one or more families from the group consisting of: flaviviruses, filoviruses, calciviruses or reoviruses. In another example, the three or more pathogens can be three or more viruses selected from gastrointestinal viruses and respiratory viruses. In another example, the three or more pathogens can be selected from the group consisting of: a pox virus, BVDV, a herpes virus, HIV, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus. The three or more pathogens can also be selected from the group consisting of: HIV, a pox virus, LCM, Junin virus, Machupo virus, Guanarito virus, Lassa Fever virus, hantavirus, Rift Valley Fever virus Ebola virus, Marburg virus or Dengue Fever virus. In another example, the three or more pathogens can be selected from the group consisting of: influenza, a pox virus, LCM, Junin virus, Machupo virus, Guanarito virus, Lassa Fever virus, hantavirus, Rift Valley Fever virus Ebola virus, Marburg virus or Dengue Fever virus

A method of identifying a compound that binds to a gene product set forth in Table 1 and can decrease infection by three or more pathogens comprising: a) contacting a compound with a gene product set forth in Table 1; b) detecting binding of the compound to the gene product; and c) associating binding with a decrease in infection by three or more pathogens. This method can further comprise optimizing a compound that binds the gene product in an assay that determines the functional ability to decrease infection by three or more pathogens. This assay can be cell based or in vivo based.

### Methods of Making Compounds

The present invention provides a method of making a compound that decreases infection of a cell by a pathogen, comprising: a) synthesizing a compound; b) administering the compound to a cell containing a cellular gene encoding a gene product set forth in Table 1; c) contacting the cell with an infectious pathogen; d) determining the level of infection, a decrease or elimination of infection indicating that the agent is an agent that decreases infection; and e) associating the agent with decreasing expression or activity of the gene product.

Further provided is a method of making a compound that decreases infection in a cell by a pathogen, comprising: a) optimizing a compound to bind a gene product set forth in Table 1; b) administering the compound to a cell containing a cellular gene encoding the gene product; c) contacting the cell with an infectious pathogen; d) determining the level of infection, a decrease or elimination of infection indicating the making of a compound that decreases infection in a cell by a pathogen.

This method can further synthesizing therapeutic quantities of the compound.

The present invention also provides a method of synthesizing a compound that binds to a gene product set forth in Table 1 and decreases infection by a pathogen comprising: a) contacting a library of compounds with a gene product set forth in Table 1; b) associating binding with a decrease in infection; and c) synthesizing derivatives of the compounds from the library that bind to the gene product.

The present invention also provides a business method to reduce the cost of drug discovery of drugs that can reduce infection by a pathogen comprising: screening, outside of the United States, for drugs that reduce infection by binding to or reducing the function of a gene product set forth in Table 1; and b) importing drugs that reduce infection into the United States. Also provided is a method of making drugs comprising directing the synthesis of drugs that reduce infection by binding to or reducing the function of a gene or gene product set forth in Table 1.

### Examples

Following infection with the U3NeoSV 1 retrovirus gene trap shuttle vector, libraries of mutagenized Vero cells were isolated in which each clone contained a single gene disrupted by provirus integration. Gene entrapment was performed essentially as described in U.S. Pat. No. 6,448,000 and U.S. Pat. No. 6,777,177. The entrapment libraries were infected with HSV, RSV, rhinovirus or Dengue fever virus and virus-resistant clones were selected as described below.

### HSV

Four days prior to infection, Vero gene trap library cells were thawed at room temperature. 13 mLs of complete growth medium and a thawed gene trap library aliquot were combined in a sterile 15 mL conical tube. This was centrifuged at 1000 rpm for 5 minutes to pellet the cells. The supernatant was discarded and the cells were resuspended in complete growth medium and the aliquot of cells seeded into 4 T150 flask. The cells were allowed to grow for 4 days at 37° C. in 5% CO₂ or until the cells were 70-100% confluent. On the day of infection, the medium in the T150 flasks was replaced with 19 mLs of fresh complete growth medium immediately before infecting the cells. One aliquot of HSV Strain 186 was thawed from the −80° C. freezer at 4° C. for 30 minutes. The HSV-2 (186 strain) was diluted in complete growth medium to a final concentration of 495 p.f.u./ml. 1 mL of diluted virus was added to each of the 4 T150 flasks containing Vero gene trap library cells. The cells were incubated at 37° C., 5% CO₂ for 2 hours. The medium was discarded from the flasks into the waste container and replaced with 20 mLs of fresh complete growth medium to remove the inoculum. The cells were incubated at 37° C., 5% CO₂. Infection was allowed to proceed without changing the medium until the cells were approximately 90% dead or dying (routinely 3 or 4 days post-infection). From then on, the medium was changed daily through day 7 post-infection. The medium was changed on days 10, 14, 17, 21, etc. post-infection. HSV-resistant colonies (clones) were observed 2-3 weeks post-infection by examining the under side of the flasks. When visible colonies appeared, they were marked and looked at under the microscope to determine which colonies are either (A) unhealthy/dying cells or are (B) actually two colonies very close together. 24-well plate(s) with 1 mL of complete growth medium in as many wells as there were resistant colonies were prepared. Resistant cells were trypsinized and cells from each HSV-resistant clone were transferred to a single well of the 24 well plate (already containing 1 ml of complete growth medium). This process was repeated for each colony. The colonies were grown until cells in several wells approach 20-30% confluency. At this point, cells were detached and seeded into duplicate 24-well plates. Resistance confirmation was performed by re-infecting clones in one 24-well plate. Following identification of resistant clones, resistant clones in the uninfected 24-well plates were expanded in T75 flasks for subsequent genomic DNA isolation (DNeasy kits, Qiagen, Inc.).

### Identification of Genes Disrupted in HSV-Resistant Clones

The U3NeoSV1 gene trap vector contains a plasmid origin of replication and ampicillin resistance gene; thus, regions of genomic DNA adjacent to the targeting vector were readily cloned by plasmid rescue and sequenced. The flanking sequences were compared to the nucleic acid databases to identify candidate cellular genes that confer resistance to lytic infection by herpes simplex virus when altered by gene entrapment.

### RSV

Four days prior to infection, Vero gene trap library cells were thawed at room temperature. 13 mLs of complete growth medium and a thawed gene trap library aliquot were combined in a sterile 15 mL conical tube. This was centrifuged at 1000 rpm for 5 minutes to pellet the cells. The supernatant was discarded and the cells were resuspended in complete growth medium and the aliquot of cells seeded into 4 T150 flask. The cells were allowed to grow for 4 days at 37° C. in 5% CO₂ or until the cells were 70-100% confluent. On the day of infection, the medium in the T150 flasks was replaced with 19 mLs of fresh complete growth medium immediately before infecting the cells. One aliquot of RSV A2 strain was thawed from the −80° C. freezer at 4° C. for 30 minutes. The RSV A2 strain was diluted in complete growth medium to a final concentration of 11,812 p.f.u./ml. 1 mL of diluted virus was added to each of the 4 T150 flasks containing Vero gene trap library cells. The cells were incubated at 37° C., 5% CO₂ for 2 hours. The medium was discarded from the flasks and replaced with 20 mLs of fresh complete growth medium to remove the inoculum. The cells were incubated at 37° C., 5% CO₂. Infection was allowed to proceed without changing the medium until the cells were approximately 90% dead or dying (approximately 3 or 4 days post-infection). From then on, the medium was changed daily through day 7 post-infection. The medium was changed on days 10, 14, 17, 21, etc. post-infection. RSV-resistant colonies (clones) were observed 2-3 weeks post-infection by examining the under side of the flasks. When visible colonies appeared, they were marked and looked at under the microscope to determine which colonies are either (A) unhealthy/dying cells or are (B) actually two colonies very close together. 24-well plate(s) with 1 mL of complete growth medium in as many wells as there were resistant colonies were prepared. Resistant cells were trypsinized and cells from each RSV-resistant clone were transferred to a single well of the 24 well plate (already containing 1 ml of complete growth medium). This process was repeated for each colony. The colonies were grown until cells in several wells approach 20-30% confluency. At this point, cells were detached and seeded into duplicate 24-well plates. Resistance confirmation was performed by re-infecting clones in one 24-well plate. Following identification of resistant clones, resistant clones in the uninfected 24-well plates were expanded in T75 flasks for subsequent genomic DNA isolation (DNeasy kits, Qiagen, Inc.).

### Identification of Genes Disrupted in RSV-Resistant Clones

The U3NeoSV1 gene trap vector contains a plasmid origin of replication and ampicillin resistance gene; thus, regions of genomic DNA adjacent to the targeting vector were readily cloned by plasmid rescue and sequenced. The flanking sequences were compared to the nucleic acid databases to identify candidate cellular genes that confer resistance to lytic infection by respiratory syncytial virus when altered by gene entrapment.

### Rhinovirus

Four days prior to infection, an aliquot of TZM-bl gene trap library cells were thawed at room temperature. 13 mLs of complete growth medium and thawed gene trap library aliquot were combined in a sterile 15 mL conical tube. This was centrifuged at 1000 rpm for 5 minutes to pellet the cells. The supernatant was discarded The cells were resuspended in complete growth medium and the aliquot of cells was seeded into 4 T150 flasks with reclosable lids. Cells were allowed grow for 4 days at 37° C. in 5% CO₂ or until the cells were 70-100% confluent. On the day of infection, the medium was replaced in the T150 flasks with 19 mLs of fresh complete growth medium immediately before infecting the cells. One aliquot of Rhinovirus-16 Strain 11757 from the −80° C. freezer was thawed at 4° C. for 30 minutes Rhinovirus was diluted in complete growth medium to a final concentration of 1×10⁵ p.f.u./ml. Approximately 1 mL of the diluted virus was added to each of 4 T150 flasks containing TZM-bl gene trap library cells. 200 uL of sterile MgCl2 was added to each T150 flask (final MgCl2 concentration=40 mM). The T150 flasks were placed on a rocker and incubated at 33° C., 5% CO₂, rocking cells gently at the lowest setting. Infection was allowed to proceed without changing the medium until the cells were >99.9% dead or dying (routinely 6-7 days post-infection). The medium was changed and the flasks transferred to a 37° C., 5% CO₂ incubator.

The medium was changed on days 10, 14, 17, 21, etc. post-infection (following this pattern of days), while maintaining cells at 37° C., 5% CO₂.

Rhinovirus resistant does were observed 2-3 weeks post-infection by examining the under side of the flasks. When visible colonies appeared, they were marked and looked at under the microscope to determine which colonies are either (A) unhealthy/dying cells or are (B) actually two colonies very close together. 24-well plate(s) with 1 mL of complete growth medium in as many wells as there were resistant colonies were prepared. Resistant cells were trypsinized and cells from each rhinovirus-resistant clone were transferred to a single well of the 24 well plate (already containing 1 ml of complete growth medium). This process was repeated for each colony. The colonies were grown until cells in several wells approach 20-30% confluency. At this point, cells were detached and seeded into duplicate 24-well plates. Resistance confirmation was performed by re-infecting clones in one 24-well plate. Following identification of resistant clones, resistant clones in the uninfected 24-well plates were expanded in T75 flasks for subsequent genomic DNA isolation (DNeasy kits, Qiagen, Inc.).

### Identification of Genes Disrupted in Rhinovirus-Resistant Clones

The U3NeoSV1 gene trap vector contains a plasmid origin of replication and ampicillin resistance gene; thus, regions of genomic DNA adjacent to the targeting vector were readily cloned by plasmid rescue and sequenced. The flanking sequences were compared to the nucleic acid databases to identify candidate cellular genes that confer resistance to lytic infection by rhinovirus when altered by gene entrapment.

siRNA and Small Molecule Studies

Any of the genes set forth in Table 1 is further analyzed by contacting cells comprising a gene set forth in Table 1 with siRNA or small molecule that targets the gene product of the gene, and any pathogen set forth herein to identify the gene as a gene involved in pathogenic infection (for example, and not to be limiting, a pox virus, BVDV, a herpes virus, HIV, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus). A decrease in viral infection indicates that the gene is a gene that is involved in pathogenic infection. This process can be performed for all of the genes set forth herein with any of the viruses, bacteria, parasites or fungi set forth herein.

siRNA Transfections can be performed as follows: Pools of 4 duplexed siRNA molecules targeting a gene of interest are reconstituted to a final working concentration of 50 uM as directed by the manufacturer (Qiagen). Twenty-four hours prior to transfection, cells are plated in 6-well dishes at 3×10⁵ cells per well, such that at the time of transfection, the cells are approximately 30% confluent. Prior to transfection, the cells are washed once with 1× phosphate buffered saline, and the medium replaced with approximately 1.8 ml antibiotic-free medium. siRNA aliquots are diluted with Opti-MEM and RNAseOUT (Invitrogen), 100 ul and 1 ul per transfection, respectively. In a separate tube, transfection reagent Lipofectamine-2000 (Invitrogen) or Oligofectamine (Invitrogen) are diluted in Opti-MEM as directed by the manufacturer. Following a 5 minute incubation at room temperature, the diluted siRNA is added to the transfection reagent mixture, and incubated for an additional 20 minutes prior to adding to independent wells of the 6-well dishes. Transfections are incubated at 37° C. for 48 hours without changing the medium.

Virus Infections: Following 48-hour transfection, medium is aspirated from 6-well plates. Viruses are diluted in the appropriate medium and 500 ul of either virus-free medium or virus dilution is added to each well, and adsorption is allowed to occur at the appropriate temperature for 1 hour. Following adsorption, inoculum is aspirated off the cells, cells are washed once with 1× phosphate buffered saline, and 2 ml growth medium is added to the cells. The infected cells are incubated for 72 hours at the appropriate temperature prior to harvesting samples for viral titration.

Viral Genomic Extractions: Seventy-hours after inoculating cells, medium is harvested from 6-well dishes and centrifuged for 2 minutes at 10,000 rpm to remove any cellular debris. 200 ul of clarified medium is added to 25 ul Proteinase K, to which 200 ul PureLink96 Viral RNA/DNA lysis buffer (Invitrogen) is added according to the manufacturer. Samples were processed and viral genomic RNA or DNA is extracted using an epMotion 5075 robotics station (Eppendorf) and the PureLink96 Viral RNA/DNA kit (Invitrogen).

cDNA and Quantitative Real-Time PCR Reactions: 3 ul of extracted viral RNA is converted to cDNA using M-MLV reverse transcriptase (Invitrogen) and AmpliTaq Gold PCR buffer (Applied Biosystems). MgCl₂, dNTPs and RNAseOUT (Invitrogen) are added to achieve a final concentration of 5 mM, 1 mM and 2 U/ul, respectively. Random hexamers (Applied Biosystems) are added to obtain 2.5 mM final concentration. Reactions are incubated at 42° C. for 1 hour, followed by heat inactivation of the reverse transcriptase at 92° C. for 10 minutes. Quantitative real-time PCR reactions are set up in 10 ul volumes using 1 ul of template cDNA or extracted viral DNA using virus-specific TaqMan probes (Applied Biosystems) and RealMasterMix (Eppendorf). 2-step reactions are allowed to proceed through 40 to 50 cycles on an ep RealPlex thermocycler (Eppendorf). Quantitative standards for real-time PCR are constructed by cloning purified amplicons into pCR2-TOPO (Invitrogen) and sequenced as necessary.

The amount of viral replication in the cells contacted with siRNA to the gene of interest is calculated and compared to the amount of viral replication in control cells that did not receive siRNA targeting the gene of interest.

1. A method of decreasing infection in a cell by a pathogen comprising decreasing expression or activity of TTC9C, AREGB, ARF4, BMPR2, or IQCG.

2. The method of claim 1, wherein infection is decreased by decreasing the replication of the pathogen.

3. The method of claim 1, wherein the pathogen is a virus.

4. The method of claim 3, wherein the virus is a respiratory virus.

5. The method of claim 4 wherein the respiratory virus is a picornavirus, an orthomyxovirus, a paramyxovirus, a coronavirus, or an adenovirus.

6. The method of claim 5, wherein the respiratory virus is selected from the group consisting of influenza virus, a pox virus, parainfluenza virus, adenovirus, measles, rhinovirus, and RSV.

7-46. (canceled)

47. A cell comprising an altered or disrupted nucleic acid encoding TTC9C, AREGB, ARF4, BMPR2, or IQCG, wherein the cell has decreased susceptibility to infection by a pathogen.

48. The cell of claim 47, wherein the pathogen is a virus and the cell is infected with a virus.

49. The cell of claim 48, wherein the virus is a respiratory virus.

50. The cell of claim 49, wherein the respiratory virus is a picornavirus, an orthomyxovirus, a paramyxovirus, a coronavirus, or an adenovirus.

51. The cell of claim 50, wherein the respiratory virus is selected from the group consisting of influenza virus, a pox virus, parainfluenza virus, adenovirus, measles, rhinovirus, and RSV.


SEQUENCE LISTING (313 sequences)

SEQ ID NO: 1 - DNA, 25 bp, Artificial Sequence
  Description of Artificial Sequence Note = synthetic construct
aaggcgggtgtaagatcaaagagat

SEQ ID NO: 2 - DNA, 25 bp, Artificial Sequence
  synthetic construct
gctcaggccattatgctgctggatt

SEQ ID NO: 3 - DNA, 25 bp, Artificial Sequence
  synthetic construct
cctgcatcaaggaggccattctctt

SEQ ID NO: 4 - DNA, 25 bp, Artificial Sequence
  synthetic construct
ccacccagaccaaccagatcttgat

SEQ ID NO: 5 - DNA, 25 bp, Artificial Sequence
  synthetic construct
acttgtgtcactaacggaccgttta

SEQ ID NO: 6 - DNA, 25 bp, Artificial Sequence
  synthetic construct
gagaaaggtgtagggatgattcttt

SEQ ID NO: 7 - DNA, 25 bp, Artificial Sequence
  synthetic construct
ggttgtcagctatccaggctcatgt

SEQ ID NO: 8 - DNA, 25 bp, Artificial Sequence
  synthetic construct
ccttgcatttcgatctccgtgatga

SEQ ID NO: 9 - DNA, 25 bp, Artificial Sequence
  synthetic construct
agctagagatcaaagtggccctgaa

SEQ ID NO: 10 - DNA, 25 bp, Artificial Sequence
  Description of Artificial Sequence Note = synthetic construct
aaggacattaaagtgggctttgtaa

SEQ ID NO: 11 - DNA, 25 bp, Artificial Sequence
  synthetic construct
gggagagacaaagcatggaatgaaa

SEQ ID NO: 12 - DNA, 25 bp, Artificial Sequence
  synthetic construct
ggctggcgcccaatctgataacttt

SEQ ID NO: 13 - DNA, 25 bp, Artificial Sequence
  synthetic construct
cattctcatgaagatgacaatccat

SEQ ID NO: 14 - DNA, 25 bp, Artificial Sequence
  synthetic construct
cagccatgaagatcccggactcata

SEQ ID NO: 15 - DNA, 25 bp, Artificial Sequence
  synthetic construct
tagtcactaccaacttcctggtgaa

SEQ ID NO: 16 - DNA, 25 bp, Artificial Sequence
  synthetic construct
caaagaggtgagtagagatagttca

SEQ ID NO: 17 - DNA, 25 bp, Artificial Sequence
  synthetic construct
tgattccagttaccgtccatctcaa

SEQ ID NO: 18 - DNA, 25 bp, Artificial Sequence
  synthetic construct
tggaagactcaaaccttcctccaaa

SEQ ID NO: 19 - DNA, 25 bp, Artificial Sequence
  synthetic construct
cctgttgtctccaccagagtcagta

SEQ ID NO: 20 - DNA, 25 bp, Artificial Sequence
  synthetic construct
agaagaaatacagtgaggagcttaa

SEQ ID NO: 21 - DNA, 25 bp, Artificial Sequence
  synthetic construct
gggcttcctggagcaaacttgaact

SEQ ID NO: 22 - DNA, 25 bp, Artificial Sequence
  synthetic construct
ccaaggaaagaggcctggtccatat

SEQ ID NO: 23 - DNA, 25 bp, Artificial Sequence
  synthetic construct
tggccatgaagtaccaccctgacaa

SEQ ID NO: 24 - DNA, 25 bp, Artificial Sequence
  synthetic construct
tcttcgggaacaaggaaatgcggat

SEQ ID NO: 25 - DNA, 25 bp, Artificial Sequence
  synthetic construct
ccaagaagctgagcgagtgtctcaa

SEQ ID NO: 26 - DNA, 25 bp, Artificial Sequence
  synthetic construct
tgctgttgtccagtctgatatgaaa

SEQ ID NO: 27 - DNA, 25 bp, Artificial Sequence
  synthetic construct
catgccctgctcaccctttacatca

SEQ ID NO: 28 - DNA, 25 bp, Artificial Sequence
  synthetic construct
gctccttcctctttgggaagatgaa

SEQ ID NO: 29 - DNA, 25 bp, Artificial Sequence
  synthetic construct
caagttacttctgattggcgactca

SEQ ID NO: 30 - DNA, 25 bp, Artificial Sequence
  synthetic construct
cccgactatgtgacgtgctacaagt

SEQ ID NO: 31 - DNA, 25 bp, Artificial Sequence
  synthetic construct
ccttccatgcttgcagtattcttaa

SEQ ID NO: 32 - DNA, 25 bp, Artificial Sequence
  synthetic construct
agtacatgcactttgggcttctgta

SEQ ID NO: 33 - DNA, 25 bp, Artificial Sequence
  synthetic construct
cctcacatgttagtaggcaggaaga

SEQ ID NO: 34 - DNA, 25 bp, Artificial Sequence
  synthetic construct
ggcccatgagatgattggaactcaa

SEQ ID NO: 35 - DNA, 25 bp, Artificial Sequence
  synthetic construct
gaggcctctaagctcgcctattgaa

SEQ ID NO: 36 - DNA, 25 bp, Artificial Sequence
  synthetic construct
cagtaaatggcagccagttgggaaa

SEQ ID NO: 37 - DNA, 25 bp, Artificial Sequence
  synthetic construct
ggattggatgctgctggcaagacaa

SEQ ID NO: 38 - DNA, 25 bp, Artificial Sequence
  synthetic construct
caggattatcccgggaactttgaca

SEQ ID NO: 39 - DNA, 25 bp, Artificial Sequence
  synthetic construct
caaagacaaagaaagtgaaccggaa

SEQ ID NO: 40 - DNA, 25 bp, Artificial Sequence
  synthetic construct
ggcgtggagttggacattgagttta

SEQ ID NO: 41 - DNA, 25 bp, Artificial Sequence
  synthetic construct
caaggcccaaccactacctgcttat

SEQ ID NO: 42 - DNA, 25 bp, Artificial Sequence
  synthetic construct
ggagagaacccagcttagactagaa

SEQ ID NO: 43 - DNA, 25 bp, Artificial Sequence
  synthetic construct
aggaataccttggatggctatggta

SEQ ID NO: 44 - DNA, 25 bp, Artificial Sequence
  synthetic construct
cgggagaaggttaaagtcaatggca

SEQ ID NO: 45 - DNA, 25 bp, Artificial Sequence
  synthetic construct
tgagtgggaaatccctgctcttaaa

SEQ ID NO: 46 - DNA, 25 bp, Artificial Sequence
  synthetic construct
cagagagaagcaggccacttctgat

SEQ ID NO: 47 - DNA, 25 bp, Artificial Sequence
  synthetic construct
tcaagggttacatgcagcaacacaa

SEQ ID NO: 48 - DNA, 25 bp, Artificial Sequence
  synthetic construct
gacactctaccagaaggcggatgat

SEQ ID NO: 49 - DNA, 25 bp, Artificial Sequence
  synthetic construct
gagccagcgcaagtggaatttcgat

SEQ ID NO: 50 - DNA, 25 bp, Artificial Sequence
  synthetic construct
cgatgaactgttatcatcaccagaa

SEQ ID NO: 51 - DNA, 25 bp, Artificial Sequence
  synthetic construct
ggtgaagagcaagtttggagctgaa

SEQ ID NO: 52 - DNA, 25 bp, Artificial Sequence
  synthetic construct
acggactctggaaccatgaacatat

SEQ ID NO: 53 - DNA, 25 bp, Artificial Sequence
  synthetic construct
agcaacgaagagaagaagaactgaa

SEQ ID NO: 54 - DNA, 25 bp, Artificial Sequence
  synthetic construct
ccagggtctgttgttgactctcgaa

SEQ ID NO: 55 - DNA, 24 bp, Artificial Sequence
  synthetic construct
ccaccaagaggaagcatgtggtga

SEQ ID NO: 56 - DNA, 25 bp, Artificial Sequence
  synthetic construct
tggctatgaagaggcatattcagtt

SEQ ID NO: 57 - DNA, 25 bp, Artificial Sequence
  synthetic construct
actcgactctcagcctgtttgataa

SEQ ID NO: 58 - DNA, 25 bp, Artificial Sequence
  synthetic construct
tggacaacagtggttccctagttta

SEQ ID NO: 59 - DNA, 25 bp, Artificial Sequence
  synthetic construct
ccgagtacttataattggcagtgga

SEQ ID NO: 60 - DNA, 25 bp, Artificial Sequence
  synthetic construct
cagcaccatggattctcagatgtta

SEQ ID NO: 61 - DNA, 25 bp, Artificial Sequence
  synthetic construct
acggcaacaagagctcgcagcttta

SEQ ID NO: 62 - DNA, 25 bp, Artificial Sequence
  synthetic construct
gaccgatccgaagatggctaccatt

SEQ ID NO: 63 - DNA, 25 bp, Artificial Sequence
  synthetic construct
gaacatggccgagcttactatgaat

SEQ ID NO: 64 - DNA, 25 bp, Artificial Sequence
  synthetic construct
gggaagtaccgagatgctgtgagta

SEQ ID NO: 65 - DNA, 24 bp, Artificial Sequence
  synthetic construct
cagatacatttgcacgggccaaga

SEQ ID NO: 66 - DNA, 25 bp, Artificial Sequence
  synthetic construct
agaaggtggatttgatccctgtgaa

SEQ ID NO: 67 - DNA, 25 bp, Artificial Sequence
  synthetic construct
gaggctgacctgtctggctttaaca

SEQ ID NO: 68 - DNA, 25 bp, Artificial Sequence
  synthetic construct
gaggctgacctgtctggctttaaca

SEQ ID NO: 69 - DNA, 25 bp, Artificial Sequence
  synthetic construct
cagaccaggaggtgagaatacttct

SEQ ID NO: 70 - DNA, 25 bp, Artificial Sequence
  synthetic construct
cgtcaaaggacaagcgggatgtcta

SEQ ID NO: 71 - DNA, 25 bp, Artificial Sequence
  synthetic construct
cagattcttcgggattggctgtatg

SEQ ID NO: 72 - DNA, 25 bp, Artificial Sequence
  synthetic construct
gagggccacattgtgacatgtgaga

SEQ ID NO: 73 - DNA, 25 bp, Artificial Sequence
  synthetic construct
ccgggtgaatcagctgtttgcacaa

SEQ ID NO: 74 - DNA, 25 bp, Artificial Sequence
  synthetic construct
ccaagctctccaagatacgagtcgt

SEQ ID NO: 75 - DNA, 25 bp, Artificial Sequence
  synthetic construct
gccgcaggtgtccatttcatctaca

SEQ ID NO: 76 - DNA, 25 bp, Artificial Sequence
  synthetic construct
tcgatggacgcattcagaaggtaat

SEQ ID NO: 77 - DNA, 25 bp, Artificial Sequence
  synthetic construct
cagtgtctactatattgataccctt

SEQ ID NO: 78 - DNA, 25 bp, Artificial Sequence
  synthetic construct
cagttgggtagattcagcttcctat

SEQ ID NO: 79 - DNA, 25 bp, Artificial Sequence
  synthetic construct
tcgagtacctgaccgcggaaattct

SEQ ID NO: 80 - DNA, 25 bp, Artificial Sequence
  synthetic construct
gagagaactctcagctggacactag

SEQ ID NO: 81 - DNA, 25 bp, Artificial Sequence
  synthetic construct
gatgccttcgtgaacagccaggaat

SEQ ID NO: 82 - DNA, 25 bp, Artificial Sequence
  synthetic construct
gatccaaagccattcccaaattata

SEQ ID NO: 83 - DNA, 25 bp, Artificial Sequence
  synthetic construct
gaagatgtttctgttggcaataaca

SEQ ID NO: 84 - DNA, 25 bp, Artificial Sequence
  synthetic construct
cagcagagatggagcatcttgtgca

SEQ ID NO: 85 - DNA, 25 bp, Artificial Sequence
  synthetic construct
cagattccctgtacctggacacgat

SEQ ID NO: 86 - DNA, 25 bp, Artificial Sequence
  synthetic construct
cccaggatcacagtcaccttctata

SEQ ID NO: 87 - DNA, 25 bp, Artificial Sequence
  synthetic construct
ccctggagaccatcgacatcaacaa

SEQ ID NO: 88 - DNA, 25 bp, Artificial Sequence
  synthetic construct
tggtgaagctggtggtgaatttcct

SEQ ID NO: 89 - DNA, 25 bp, Artificial Sequence
  synthetic construct
gggaggaaatatggctccaatgtca

SEQ ID NO: 90 - DNA, 25 bp, Artificial Sequence
  synthetic construct
ccaagcagttgtgtattccaggaaa

SEQ ID NO: 91 - DNA, 25 bp, Artificial Sequence
  synthetic construct
cctggaccttggagctgatgtcaat

SEQ ID NO: 92 - DNA, 25 bp, Artificial Sequence
  synthetic construct
aagatggactcaggtggactccaaa

SEQ ID NO: 93 - DNA, 25 bp, Artificial Sequence
  synthetic construct
tgcgcctttcctgaaccgactagtt

SEQ ID NO: 94 - DNA, 25 bp, Artificial Sequence
  synthetic construct
ccagcctcatctcctttcttgctag

SEQ ID NO: 95 - DNA, 25 bp, Artificial Sequence
  synthetic construct
gaggaagccagaatcaacagctcca

SEQ ID NO: 96 - DNA, 25 bp, Artificial Sequence
  synthetic construct
tggacatcggtggaacgctggttaa

SEQ ID NO: 97 - DNA, 25 bp, Artificial Sequence
  synthetic construct
tggacatcggtggaacgctggttaa

SEQ ID NO: 98 - DNA, 25 bp, Artificial Sequence
  synthetic construct
gaagagattgtggacggcaacgcaa

SEQ ID NO: 99 - DNA, 24 bp, Artificial Sequence
  synthetic construct
catggcatgaagagggacagcaaa

SEQ ID NO: 100 - DNA, 25 bp, Artificial Sequence
  synthetic construct
catggcactgaagagggacagcaaa

SEQ ID NO: 101 - DNA, 25 bp, Artificial Sequence
  synthetic construct
gctgttccttcaagtagcacctcta

SEQ ID NO: 102 - DNA, 25 bp, Artificial Sequence
  synthetic construct
cggtgaagacacgaatgcagagttt

SEQ ID NO: 103 - DNA, 25 bp, Artificial Sequence
  synthetic construct
acctgtgtgccacttgccaatgcaa

SEQ ID NO: 104 - DNA, 25 bp, Artificial Sequence
  synthetic construct
tgtatgatgacaactcggtaatgct

SEQ ID NO: 105 - DNA, 25 bp, Artificial Sequence
  synthetic construct
tcttcgtgggagggctcaactttaa

SEQ ID NO: 106 - DNA, 25 bp, Artificial Sequence
  synthetic construct
cattgccaagctggcagagacactt

SEQ ID NO: 107 - DNA, 25 bp, Artificial Sequence
  synthetic construct
cccaatggatctttatgcaagtatt

SEQ ID NO: 108 - DNA, 25 bp, Artificial Sequence
  synthetic construct
cagagcaaataagcatggacctcaa

SEQ ID NO: 109 - DNA, 25 bp, Artificial Sequence
  synthetic construct
cgagatgctgcaatttgtcagtaat

SEQ ID NO: 110 - DNA, 25 bp, Artificial Sequence
  synthetic construct
cagattcatcttggaggcatagtca

SEQ ID NO: 111 - DNA, 25 bp, Artificial Sequence
  synthetic construct
gggtgtgtcagaagacggtttactt

SEQ ID NO: 112 - DNA, 25 bp, Artificial Sequence
  synthetic construct
caggagaggtagagaagctcctgaa

SEQ ID NO: 113 - DNA, 25 bp, Artificial Sequence
  synthetic construct
cagtggtccagctgcaacatcttct

SEQ ID NO: 114 - DNA, 25 bp, Artificial Sequence
  synthetic construct
caggatagaggagatggatcactta

SEQ ID NO: 115 - DNA, 25 bp, Artificial Sequence
  synthetic construct
aggagctacagaacgaggtagagaa

SEQ ID NO: 116 - DNA, 25 bp, Artificial Sequence
  synthetic construct
gccaactctgtgggcagaatgctaa

SEQ ID NO: 117 - DNA, 25 bp, Artificial Sequence
  synthetic construct
gggaccagttcaatgttctcatcaa

SEQ ID NO: 118 - DNA, 25 bp, Artificial Sequence
  synthetic construct
caagtgaagacatagaccaaatgtt

SEQ ID NO: 119 - DNA, 25 bp, Artificial Sequence
  synthetic construct
cccgaactctgaaggagaaactgta

SEQ ID NO: 120 - DNA, 25 bp, Artificial Sequence
  synthetic construct
caagacagcggaatcagaaagtttg

SEQ ID NO: 121 - DNA, 25 bp, Artificial Sequence
  synthetic construct
cctcgtctggcaacgatgatgatct

SEQ ID NO: 122 - DNA, 25 bp, Artificial Sequence
  synthetic construct
cagatgatgaggatgtcctagacaa

SEQ ID NO: 123 - DNA, 25 bp, Artificial Sequence
  synthetic construct
tcccaacctgaagactgcatttgca

SEQ ID NO: 124 - DNA, 25 bp, Artificial Sequence
  synthetic construct
cagattctcacaatggccaacccta

SEQ ID NO: 125 - DNA, 25 bp, Artificial Sequence
  synthetic construct
tagtgaagccgggactgccttcatt

SEQ ID NO: 126 - DNA, 25 bp, Artificial Sequence
  synthetic construct
cccacttcttcaacttcctgctcaa

SEQ ID NO: 127 - DNA, 25 bp, Artificial Sequence
  synthetic construct
aaagctctacttctctcgatgctaa

SEQ ID NO: 128 - DNA, 25 bp, Artificial Sequence
  synthetic construct
agaacctgctacggtacggactctt

SEQ ID NO: 129 - DNA, 25 bp, Artificial Sequence
  synthetic construct
cctctgcgagttgctgaatgctata

SEQ ID NO: 130 - DNA, 25 bp, Artificial Sequence
  synthetic construct
acactcggaggaaactgcaagggaa

SEQ ID NO: 131 - DNA, 25 bp, Artificial Sequence
  synthetic construct
gaggatgctcaggtcttcatccaaa

SEQ ID NO: 132 - DNA, 25 bp, Artificial Sequence
  synthetic construct
caacaaagagcaagcacctagtgat

SEQ ID NO: 133 - DNA, 25 bp, Artificial Sequence
  synthetic construct
caaggagccacaattggagttgatt

SEQ ID NO: 134 - DNA, 25 bp, Artificial Sequence
  synthetic construct
tccttggtagtgtacgcagcctgtt

SEQ ID NO: 135 - DNA, 25 bp, Artificial Sequence
  synthetic construct
gagtgcctatgtgactgtgacttat

SEQ ID NO: 136 - DNA, 25 bp, Artificial Sequence
  synthetic construct
tcgttgcattcaatgaccaacttca

SEQ ID NO: 137 - DNA, 25 bp, Artificial Sequence
  synthetic construct
tgtcagggtgggagtcatattacaa

SEQ ID NO: 138 - DNA, 20 bp, Artificial Sequence
  synthetic construct
tcttacacccgcctttccca

SEQ ID NO: 139 - DNA, 21 bp, Artificial Sequence
  synthetic construct
ggctctcattggtccttcgca

SEQ ID NO: 140 - DNA, 20 bp, Artificial Sequence
  synthetic construct
ctgttccttgtgcctctcca

SEQ ID NO: 141 - DNA, 20 bp, Artificial Sequence
  synthetic construct
acatctctccacccttccct

SEQ ID NO: 142 - DNA, 20 bp, Artificial Sequence
  synthetic construct
gggtgtgactctctctgggt

SEQ ID NO: 143 - DNA, 20 bp, Artificial Sequence
  synthetic construct
cctgccactatttcctccct

SEQ ID NO: 144 - DNA, 20 bp, Artificial Sequence
  synthetic construct
acaccatcacaggcaccaca

SEQ ID NO: 145 - DNA, 21 bp, Artificial Sequence
  synthetic construct
ccttcctttgttccctggtgc

SEQ ID NO: 146 - DNA, 20 bp, Artificial Sequence
  synthetic construct
gtcgctctcctttccttccc

SEQ ID NO: 147 - DNA, 21 bp, Artificial Sequence
  synthetic construct
cctgttcattccgcctttccc

SEQ ID NO: 148 - DNA, 21 bp, Artificial Sequence
  synthetic construct
ctctgtcccaactcctcctct

SEQ ID NO: 149 - DNA, 20 bp, Artificial Sequence
  synthetic construct
gctttccgtccacaccatct

SEQ ID NO: 150 - DNA, 20 bp, Artificial Sequence
  synthetic construct
tcccacgtcagcctccagat

SEQ ID NO: 151 - DNA, 21 bp, Artificial Sequence
  synthetic construct
tcttcttcccataactcctct

SEQ ID NO: 152 - DNA, 20 bp, Artificial Sequence
  synthetic construct
ggtggtggtggtggtggttt

SEQ ID NO: 153 - DNA, 21 bp, Artificial Sequence
  synthetic construct
gtctctgtttcccttgtgtct

SEQ ID NO: 154 - DNA, 20 bp, Artificial Sequence
  synthetic construct
tccctgttgctttccttcct

SEQ ID NO: 155 - DNA, 21 bp, Artificial Sequence
  synthetic construct
gttccactatttccattccca

SEQ ID NO: 156 - DNA, 20 bp, Artificial Sequence
  synthetic construct
gtccattacacctccagcag

SEQ ID NO: 157 - DNA, 21 bp, Artificial Sequence
  synthetic construct
gggtatccgacaattaaagtc

SEQ ID NO: 158 - DNA, 20 bp, Artificial Sequence
  synthetic construct
gaccacagccttccagacat

SEQ ID NO: 159 - DNA, 19 bp, Artificial Sequence
  synthetic construct
agtccttcccacctccaga

SEQ ID NO: 160 - DNA, 20 bp, Artificial Sequence
  synthetic construct
ttctccacctcagccgtcag

SEQ ID NO: 161 - DNA, 21 bp, Artificial Sequence
  synthetic construct
tcccactcactgctttctcct

SEQ ID NO: 162 - DNA, 21 bp, Artificial Sequence
  synthetic construct
gtcttcttgttgtcccgagcc

SEQ ID NO: 163 - DNA, 21 bp, Artificial Sequence
  synthetic construct
gtgtcctaagaaatgccatca

SEQ ID NO: 164 - DNA, 19 bp, Artificial Sequence
  synthetic construct
agtcatcacagcaaccaca

SEQ ID NO: 165 - DNA, 20 bp, Artificial Sequence
  synthetic construct
gccactcctgcttcttcgtc

SEQ ID NO: 166 - DNA, 20 bp, Artificial Sequence
  synthetic construct
gcacagtctctcccttagtt

SEQ ID NO: 167 - DNA, 20 bp, Artificial Sequence
  synthetic construct
gccaccacctcagaagacct

SEQ ID NO: 168 - DNA, 20 bp, Artificial Sequence
  synthetic construct
tctctccagcccaactctct

SEQ ID NO: 169 - DNA, 20 bp, Artificial Sequence
  synthetic construct
actcctcctcccagttctgc

SEQ ID NO: 170 - DNA, 20 bp, Artificial Sequence
  synthetic construct
agtctcacccaaccaccctg

SEQ ID NO: 171 - DNA, 21 bp, Artificial Sequence
  synthetic construct
gccttccagagccacatcatc

SEQ ID NO: 172 - DNA, 21 bp, Artificial Sequence
  synthetic construct
cctatgccacttggacagagc

SEQ ID NO: 173 - DNA, 21 bp, Artificial Sequence
  synthetic construct
cctatgccacttggacagagc

SEQ ID NO: 174 - DNA, 19 bp, Artificial Sequence
  synthetic construct
ccttgtccctctgcttctc

SEQ ID NO: 175 - DNA, 21 bp, Artificial Sequence
  synthetic construct
gactcccaaattccaccaccc

SEQ ID NO: 176 - DNA, 20 bp, Artificial Sequence
  synthetic construct
ccttgtgtccctttcgtccc

SEQ ID NO: 177 - DNA, 20 bp, Artificial Sequence
  synthetic construct
agcagccatatcccaagccc

SEQ ID NO: 178 - DNA, 20 bp, Artificial Sequence
  synthetic construct
gcattccacctccaccatcc

SEQ ID NO: 179 - DNA, 20 bp, Artificial Sequence
  synthetic construct
tcccgtgatccacttccact

SEQ ID NO: 180 - DNA, 20 bp, Artificial Sequence
  synthetic construct
gtgtgttcttcatcttcctc

SEQ ID NO: 181 - DNA, 20 bp, Artificial Sequence
  synthetic construct
tctgtccctcctccttccct

SEQ ID NO: 182 - DNA, 21 bp, Artificial Sequence
  synthetic construct
ctctttctcttccaccctccc

SEQ ID NO: 183 - DNA, 20 bp, Artificial Sequence
  synthetic construct
agtcctcagccttgtcccac

SEQ ID NO: 184 - DNA, 21 bp, Artificial Sequence
  synthetic construct
cttccctctcccaaatccacc

SEQ ID NO: 185 - DNA, 20 bp, Artificial Sequence
  synthetic construct
tcttgaccaccaacatccca

SEQ ID NO: 186 - DNA, 21 bp, Artificial Sequence
  synthetic construct
cctctccctctctctctctct

SEQ ID NO: 187 - DNA, 20 bp, Artificial Sequence
  synthetic construct
ctccacccgcacactctctt

SEQ ID NO: 188 - DNA, 20 bp, Artificial Sequence
  synthetic construct
tcctcccacctcaacctcct

SEQ ID NO: 189 - DNA, 20 bp, Artificial Sequence
  synthetic construct
ctccctctgtgactcctcgt

SEQ ID NO: 190 - DNA, 20 bp, Artificial Sequence
  synthetic construct
cctctgtcctccctcttctc

SEQ ID NO: 191 - DNA, 20 bp, Artificial Sequence
  synthetic construct
gccaccttcccttcctctca

SEQ ID NO: 192 - DNA, 21 bp, Artificial Sequence
  synthetic construct
tcttgtcctccctaatctcct

SEQ ID NO: 193 - DNA, 20 bp, Artificial Sequence
  synthetic construct
tcccaactccaccatcaccc

SEQ ID NO: 194 - DNA, 20 bp, Artificial Sequence
  synthetic construct
gttccttgtctcccacctcc

SEQ ID NO: 195 - DNA, 21 bp, Artificial Sequence
  synthetic construct
gtagtctcgccatttgtccct

SEQ ID NO: 196 - DNA, 20 bp, Artificial Sequence
  synthetic construct
cccttctccacctcttgcca

SEQ ID NO: 197 - DNA, 19 bp, Artificial Sequence
  synthetic construct
tttcaccctctaccctccc

SEQ ID NO: 198 - DNA, 21 bp, Artificial Sequence
  synthetic construct
gctgtcttcatcctctggctc

SEQ ID NO: 199 - DNA, 20 bp, Artificial Sequence
  synthetic construct
actcctctcacttcccgcct

SEQ ID NO: 200 - DNA, 20 bp, Artificial Sequence
  synthetic construct
atctccctctccctcctcca

SEQ ID NO: 201 - DNA, 20 bp, Artificial Sequence
  synthetic construct
tcccgtggttggctccttgt

SEQ ID NO: 202 - DNA, 21 bp, Artificial Sequence
  synthetic construct
gacttcaccaatcccagccag

SEQ ID NO: 203 - DNA, 21 bp, Artificial Sequence
  synthetic construct
cctcctcttcactctcctcct

SEQ ID NO: 204 - DNA, 21 bp, Artificial Sequence
  synthetic construct
tctcccgtttaccacagccct

SEQ ID NO: 205 - DNA, 20 bp, Artificial Sequence
  synthetic construct
gtccttcactcccatccacc

SEQ ID NO: 206 - DNA, 20 bp, Artificial Sequence
  synthetic construct
gtccactccctccatcacca

SEQ ID NO: 207 - DNA, 20 bp, Artificial Sequence
  synthetic construct
tgatcctcccacctcagcct

SEQ ID NO: 208 - DNA, 20 bp, Artificial Sequence
  synthetic construct
tgatcctcccacctcagcct

SEQ ID NO: 209 - DNA, 21 bp, Artificial Sequence
  synthetic construct
gtcttccacacagaactaggc

SEQ ID NO: 210 - DNA, 20 bp, Artificial Sequence
  synthetic construct
gttcccttcctccactgcca

SEQ ID NO: 211 - DNA, 21 bp, Artificial Sequence
  synthetic construct
tctggctctggtggtggttct

SEQ ID NO: 212 - DNA, 20 bp, Artificial Sequence
  synthetic construct
tgctgctgttcccaccctct

SEQ ID NO: 213 - DNA, 20 bp, Artificial Sequence
  synthetic construct
gtcttgctctccctctcgtc

SEQ ID NO: 214 - DNA, 20 bp, Artificial Sequence
  synthetic construct
gtcctcctcctcctcctctt

SEQ ID NO: 215 - DNA, 21 bp, Artificial Sequence
  synthetic construct
gctggttcccttcctccttgt

SEQ ID NO: 216 - DNA, 20 bp, Artificial Sequence
  synthetic construct
cctccacttccgcctttcag

SEQ ID NO: 217 - DNA, 20 bp, Artificial Sequence
  synthetic construct
cctccacttccgcctttcag

SEQ ID NO: 218 - DNA, 21 bp, Artificial Sequence
  synthetic construct
tctctgttccctccgtctcct

SEQ ID NO: 219 - DNA, 20 bp, Artificial Sequence
  synthetic construct
ccatcctgctcttctccacc

SEQ ID NO: 220 - DNA, 20 bp, Artificial Sequence
  synthetic construct
acctcccacctctccttaca

SEQ ID NO: 221 - DNA, 20 bp, Artificial Sequence
  synthetic construct
ggctgtgtgggtggagtgta

SEQ ID NO: 222 - DNA, 20 bp, Artificial Sequence
  synthetic construct
tctccctcgcccaactctct

SEQ ID NO: 223 - DNA, 21 bp, Artificial Sequence
  synthetic construct
gtcccattccacgtcctcttc

SEQ ID NO: 224 - DNA, 20 bp, Artificial Sequence
  synthetic construct
tgcctctgtgaccctcccta

SEQ ID NO: 225 - DNA, 20 bp, Artificial Sequence
  synthetic construct
ggctcttgtcttcttgggtc

SEQ ID NO: 226 - DNA, 20 bp, Artificial Sequence
  synthetic construct
tcctctccttgtgtttccct

SEQ ID NO: 227 - DNA, 20 bp, Artificial Sequence
  synthetic construct
cctccttcttcttctcttct

SEQ ID NO: 228 - DNA, 20 bp, Artificial Sequence
  synthetic construct
gccagctcctccctcatctt

SEQ ID NO: 229 - DNA, 20 bp, Artificial Sequence
  synthetic construct
cctgccttcctttctgttgt

SEQ ID NO: 230 - DNA, 19 bp, Artificial Sequence
  synthetic construct
tctcttctccctcctgcca

SEQ ID NO: 231 - DNA, 21 bp, Artificial Sequence
  synthetic construct
gtcttcttgttgtcccgagcc

SEQ ID NO: 232 - DNA, 20 bp, Artificial Sequence
  synthetic construct
ttcttgttcccacccaggca

SEQ ID NO: 233 - DNA, 21 bp, Artificial Sequence
  synthetic construct
ccaccgttccttctcttccct

SEQ ID NO: 234 - DNA, 20 bp, Artificial Sequence
  synthetic construct
gtcccaagtccagaggcagt

SEQ ID NO: 235 - DNA, 20 bp, Artificial Sequence
  synthetic construct
tctccacctcctcctctcca

SEQ ID NO: 236 - DNA, 21 bp, Artificial Sequence
  synthetic construct
tctcccacccttccttactcc

SEQ ID NO: 237 - DNA, 19 bp, Artificial Sequence
  synthetic construct
atgtccacgtcctccacct

SEQ ID NO: 238 - DNA, 21 bp, Artificial Sequence
  synthetic construct
gtttcttccttgtatttgccc

SEQ ID NO: 239 - DNA, 21 bp, Artificial Sequence
  synthetic construct
ccttctatgtcccttcccttc

SEQ ID NO: 240 - DNA, 20 bp, Artificial Sequence
  synthetic construct
ctctctctctccctctctct

SEQ ID NO: 241 - DNA, 20 bp, Artificial Sequence
  synthetic construct
tcctcctcgtttcgccacct

SEQ ID NO: 242 - DNA, 20 bp, Artificial Sequence
  synthetic construct
gcctccatccactcctgaca

SEQ ID NO: 243 - DNA, 21 bp, Artificial Sequence
  synthetic construct
cctcctcaccacacattccct

SEQ ID NO: 244 - DNA, 21 bp, Artificial Sequence
  synthetic construct
cctcctcaccacacattccct

SEQ ID NO: 245 - DNA, 20 bp, Artificial Sequence
  synthetic construct
acacctgccacccacttcct

SEQ ID NO: 246 - DNA, 21 bp, Artificial Sequence
  synthetic construct
tcctcctcctcctcctcttct

SEQ ID NO: 247 - DNA, 20 bp, Artificial Sequence
  synthetic construct
cttcctcccagccactcact

SEQ ID NO: 248 - DNA, 21 bp, Artificial Sequence
  synthetic construct
gttcctgcacattccctctcc

SEQ ID NO: 249 - DNA, 20 bp, Artificial Sequence
  synthetic construct
cttcctcccagccactcact

SEQ ID NO: 250 - DNA, 21 bp, Artificial Sequence
  synthetic construct
catcaatacttcctggctccc

SEQ ID NO: 251 - DNA, 20 bp, Artificial Sequence
  synthetic construct
ccctcccttcctctccactt

SEQ ID NO: 252 - DNA, 19 bp, Artificial Sequence
  synthetic construct
cttgaactccatgccacaa

SEQ ID NO: 253 - DNA, 20 bp, Artificial Sequence
  synthetic construct
gcttccttcccgtcagtcca

SEQ ID NO: 254 - DNA, 21 bp, Artificial Sequence
  synthetic construct
ccaccttctcctttgcctcct

SEQ ID NO: 255 - DNA, 20 bp, Artificial Sequence
  synthetic construct
cctaccctccttccatccct

SEQ ID NO: 256 - DNA, 20 bp, Artificial Sequence
  synthetic construct
tgtctcccttcccagccaca

SEQ ID NO: 257 - DNA, 21 bp, Artificial Sequence
  synthetic construct
gtcatcctttcttctcctccc

SEQ ID NO: 258 - DNA, 20 bp, Artificial Sequence
  synthetic construct
gtccttcctgtccaccacca

SEQ ID NO: 259 - DNA, 20 bp, Artificial Sequence
  synthetic construct
cttgtgccagttgtcccgag

SEQ ID NO: 260 - DNA, 21 bp, Artificial Sequence
  synthetic construct
gctctcatctctctcccttca

SEQ ID NO: 261 - DNA, 20 bp, Artificial Sequence
  synthetic construct
ctcctcagacacactttatc

SEQ ID NO: 262 - DNA, 20 bp, Artificial Sequence
  synthetic construct
gccacaaccacgcctaagga

SEQ ID NO: 263 - DNA, 19 bp, Artificial Sequence
  synthetic construct
gctcagctctccaaggttg

SEQ ID NO: 264 - DNA, 20 bp, Artificial Sequence
  synthetic construct
tacctgccactcccatagcc

SEQ ID NO: 265 - DNA, 21 bp, Artificial Sequence
  synthetic construct
tcctcctcctcctcctcttct

SEQ ID NO: 266 - DNA, 20 bp, Artificial Sequence
  synthetic construct
ttctcctgtctctgcctccc

SEQ ID NO: 267 - DNA, 20 bp, Artificial Sequence
  synthetic construct
cacctcctcttccctgtcca

SEQ ID NO: 268 - DNA, 20 bp, Artificial Sequence
  synthetic construct
ccctgccacctatcctctca

SEQ ID NO: 269 - DNA, 20 bp, Artificial Sequence
  synthetic construct
tctcttccctctcgcctcct

SEQ ID NO: 270 - DNA, 21 bp, Artificial Sequence
  synthetic construct
ggttgtgtgtggtgtggttct

SEQ ID NO: 271 - DNA, 20 bp, Artificial Sequence
  synthetic construct
ttcgctccctgtcctcctct

SEQ ID NO: 272 - DNA, 20 bp, Artificial Sequence
  synthetic construct
tactctgttcccgctgccac

SEQ ID NO: 273 - DNA, 20 bp, Artificial Sequence
  synthetic construct
tcctcctctccaccaccttc

SEQ ID NO: 274 - DNA, 21 bp, Artificial Sequence
  synthetic construct
tcccacttcctcccattccct

SEQ ID NO: 275 - DNA, 20 bp, Artificial Sequence
  synthetic construct
tcctccacctcccgttgctt

SEQ ID NO: 276 - DNA, 19 bp, Artificial Sequence
  synthetic construct
gtccctggctgttgtgagt

SEQ ID NO: 277 - DNA, 20 bp, Artificial Sequence
  synthetic construct
cactcatcatgcccgtccct

SEQ ID NO: 278 - DNA, 20 bp, Artificial Sequence
  synthetic construct
tccactcccaatgctctccc

SEQ ID NO: 279 - DNA, 21 bp, Artificial Sequence
  synthetic construct
gtcgtccctttcactcccatc

SEQ ID NO: 280 - DNA, 20 bp, Artificial Sequence
  synthetic construct
gcagttcaccaccagctctc

SEQ ID NO: 281 - DNA, 21 bp, Artificial Sequence
  synthetic construct
ccacagcacacctctttccct

SEQ ID NO: 282 - DNA, 21 bp, Artificial Sequence
  synthetic construct
gtctctctttcttgctccctg

SEQ ID NO: 283 - DNA, 20 bp, Artificial Sequence
  synthetic construct
ccaccaccgtcttcttgcca

SEQ ID NO: 284 - DNA, 20 bp, Artificial Sequence
  synthetic construct
gttctttcccttctctccca

SEQ ID NO: 285 - DNA, 21 bp, Artificial Sequence
  synthetic construct
cccttcttcccttctctgtgt

SEQ ID NO: 286 - DNA, 20 bp, Artificial Sequence
  synthetic construct
actctcctcctctccatcct

SEQ ID NO: 287 - DNA, 20 bp, Artificial Sequence
  synthetic construct
cctcctcttctaccgcttct

SEQ ID NO: 288 - DNA, 21 bp, Artificial Sequence
  synthetic construct
gccatttgtcgtcctcttccc

SEQ ID NO: 289 - DNA, 20 bp, Artificial Sequence
  synthetic construct
cgttcctcctctgccacaca

SEQ ID NO: 290 - DNA, 21 bp, Artificial Sequence
  synthetic construct
tccctgcttactccttaccct

SEQ ID NO: 291 - DNA, 20 bp, Artificial Sequence
  synthetic construct
gcgtctctatttcctccctg

SEQ ID NO: 292 - DNA, 20 bp, Artificial Sequence
  synthetic construct
attctctgccaccctgtccc

SEQ ID NO: 293 - DNA, 20 bp, Artificial Sequence
  synthetic construct
tcctgccattactccctccg

SEQ ID NO: 294 - DNA, 20 bp, Artificial Sequence
  synthetic construct
gtcaattccagccaccagag

SEQ ID NO: 295 - DNA, 19 bp, Artificial Sequence
  synthetic construct
acagggtcgatctgatggg

SEQ ID NO: 296 - DNA, 21 bp, Artificial Sequence
  synthetic construct
gctcaagctggcatatcagac

SEQ ID NO: 297 - DNA, 20 bp, Artificial Sequence
  synthetic construct
agcattgtcgatctgatggg

SEQ ID NO: 298 - DNA, 20 bp, Artificial Sequence
  synthetic construct
cctcaccacctcttcctcct

SEQ ID NO: 299 - DNA, 20 bp, Artificial Sequence
  synthetic construct
tccctgcctcacgattccct

SEQ ID NO: 300 - DNA, 21 bp, Artificial Sequence
  synthetic construct
tccctgtcacttctctggctc

SEQ ID NO: 301 - DNA, 20 bp, Artificial Sequence
  synthetic construct
ttcctttgtgcccttcccag

SEQ ID NO: 302 - DNA, 20 bp, Artificial Sequence
  synthetic construct
cctctgtgctcgttcctctc

SEQ ID NO: 303 - DNA, 19 bp, Artificial Sequence
  synthetic construct
gagttcctttcccagccct

SEQ ID NO: 304 - DNA, 20 bp, Artificial Sequence
  synthetic construct
gctctctcttgacctcctcc

SEQ ID NO: 305 - DNA, 21 bp, Artificial Sequence
  synthetic construct
actcctcttgccacttgtcca

SEQ ID NO: 306 - DNA, 21 bp, Artificial Sequence
  synthetic construct
tctatctcccgcagccactca

SEQ ID NO: 307 - DNA, 21 bp, Artificial Sequence
  synthetic construct
gtcccttagagcaacccatac

SEQ ID NO: 308 - DNA, 20 bp, Artificial Sequence
  synthetic construct
cctaccactcccatagcatt

SEQ ID NO: 309 - DNA, 20 bp, Artificial Sequence
  synthetic construct
gccaccatgactccctcctt

SEQ ID NO: 310 - DNA, 20 bp, Artificial Sequence
  synthetic construct
tttcccagactcaccagccc

SEQ ID NO: 311 - DNA, 21 bp, Artificial Sequence
  synthetic construct
ctttctccttccttccagtcc

SEQ ID NO: 312 - DNA, 21 bp, Artificial Sequence
  synthetic construct
tctccacagtgtcttctccca

SEQ ID NO: 313 - DNA, 21 bp, Artificial Sequence
  synthetic construct
tcttcagcccactcttcagcc